(19) United States
(12) Patent Application Publication
(10) Pub. No.: US 2026/0209795 A1
(43) Pub. Date: Jul. 23, 2026

(54) MATERIALS AND METHODS FOR CHARACTERIZING POTENCY

(71) Applicant: ENCODED THERAPEUTICS, INC., South San Francisco, CA (US)

(72) Inventors: Anne Tanenhaus, South San Francisco, CA (US); Sirika Wood, South San Francisco, CA (US); Chi Zhang, Sunnyvale, CA (US); Dhruv Bole, South San Francisco, CA (US); Tiffany Cai, South San Francisco, CA (US); Steven Tan, South San Francisco, CA (US)

(21) Appl. No.: 19/140,841

(22) PCT Filed: Dec. 28, 2023
(86) PCT No.: PCT/US2023/086270

§ 371 Date: Jun. 18, 2025

Related U.S. Application Data
(60) Provisional application No. 63/436,030, filed on Dec. 29, 2022.

(51) Int. Cl. C12N 15/86; C07K 14/435; C07K 14/705; C12N 9/02; C12Q 1/6897

(52) U.S. Cl. CPC C12N 15/86; C07K 14/43595; C07K 14/705; C12N 9/0069; C12Q 1/6897; C12Y 113/12007; C12N 2750/14143

(57) ABSTRACT

The disclosure provides a nucleic acid comprising a response element operably linked to a reporter nucleotide sequence. In various embodiments, the response element comprises from 2 to 10 copies of a Z1 transcription factor binding site. Cells comprising the nucleic acid also are provided. In various aspects, the cells stably overexpress an adeno-associated virus receptor. The disclosure further provides a method of determining the potency of a sample comprising AAV vectors.

### TECHNICAL FIELD

The disclosure relates to nucleic acids, cells, and methods for characterizing the potency of samples comprising AAV vectors.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: file name; “55328A_SeqListing.XML,” 126,534 bytes, created on Dec. 28, 2023.

### BACKGROUND

Pharmaceutics and biopharmaceuticals approved by regulatory agencies must be accompanied by analytical assays to confirm that product lots meet defined criteria (purity, safety, potency, etc.) deemed suitable for therapeutics approved for human use. Potency assays and related tests are used to ensure consistent quality among product lots and are used to assure identity, purity, strength (potency), and stability of products during studies. Analytical assays have long been used to characterize small molecule pharmaceutical products and protein-based therapeutics, such as monoclonal antibodies. The nature of gene therapy products complicates the development of potency assays, however. Several characteristics of gene and cell therapies pose significant challenges in developing potency assays, including an inherent variability of starting materials, lack of appropriate reference standards, complex mechanism of action(s), and the in vivo fate of product. As the number of gene therapy candidates increase, the industry continues to struggle in developing assays that reliably quantify potency of gene therapy product samples.

### SUMMARY

The present disclosure provides materials and methods for measuring the potency of expression vector delivery vehicles. For example, the disclosure provides a nucleic acid comprising a response element (RE) operably linked to a reporter nucleotide sequence. In various embodiments, the response element comprises from 2 to 10 copies (e.g., 3 to 8 or 3 to 6 copies) of a Z1 transcription factor (TF) binding site. Optionally, the Z1 TF binding site has a nucleic acid sequence of SEQ ID NO: 1 or a nucleic acid sequence having 1, 2, or 3 nucleotide differences from SEQ ID NO:1. In various embodiments, the response element comprises a spacer sequence (e.g., a sequence of SEQ ID NO: 2 or SEQ ID NO: 3) between at least two copies of the Z1 TF binding site. The response element may further comprise a promoter, such as a minimal promoter (e.g., SEQ ID NO: 32) and/or a polyA signal sequence. In various aspects of the disclosure, the reporter nucleotide sequence encodes a reporter protein which may identified and measured. Examples of reporter proteins include luminescent proteins or enzymes that produce bioluminescence, such as luciferase; fluorescent proteins such as green fluorescent protein (GFP), enhanced GFP (EGFP), and mCherry; and colored proteins or enzymes which produce a colored product such as beta-galactosidase. The nucleic acid may, in some embodiments, comprise a second reporter nucleotide sequence operably linked to a constitutive promoter.

A cell comprising the nucleic acid also is provided. For instance, the disclosure provides a cell comprising a nucleic acid comprising a response element (e.g., a response element comprises from 2 to 10 copies (e.g., 3 to 8 or 3 to 6 copies) of a transcription factor (TF) binding site) operably linked to a reporter nucleotide sequence, wherein the cell is further engineered to stably overexpress an adeno-associated virus receptor (AAVR), such as wild-type AAVR. Optionally, the response element comprises one or more TF binding site(s) capable of being bound by a TF. In various aspects, the TF is a ligand-dependent TF, such as a metal (e.g., copper) dependent TF. Alternatively, the response element comprises one or more TF binding site(s) bound by an exogenous TF. In various aspects, the exogenous TF comprises an engineered DNA binding domain specific for the TF binding site, such as an engineered DNA binding site comprising from 2 to 10 zinc fingers.

The disclosure further provides a method of determining the potency of a sample comprising AAV vectors. The method comprises contacting a cell comprising a response element operably linked to a reporter nucleotide sequence with all or part of the sample. The AAV vector encodes a modulator that directly or indirectly modulates expression of the reporter nucleotide sequence via the response element. The method further comprises measuring the expression of the reporter nucleotide sequence in the cell. The method further optionally comprises determining the potency of the sample based on the measured expression level of the reporter nucleic acid sequence.

### BRIEF DESCRIPTION OF THE DRAWINGS

FIGS. 1A-1D are schematics of nucleic acids comprising a response element operably linked to a reporter nucleotide sequence.

FIG. 2 illustrates reporter expression (measured as relative light units (RLU; y-axis)) of various response element constructs in the presence and in the absence of a modulator (SEQ ID NO: 34). The presence of reporter only is represented by bar on left for each response element construct; reporter+activator is represented by bar on right for each response element construct.

FIG. 3 illustrates reporter expression (measured as relative light units (RLU; y-axis)) of various response element constructs in the presence of increasing amounts of a modulator (SEQ ID NO: 34; 0 ng, 0.01 ng, 0.1 ng, 1 ng, 10 ng, 30 ng, 50 ng, or 70 ng).

FIG. 4 illustrates the results of Example 3. FIG. 4 illustrates reporter expression (measured as relative light units (RLU; y-axis)) observed from a control expression cassette having TET reporter sequence in place of a DNA binding sequence (P-1) and a cassette comprising a Z1-based response element operably linked to a reporter nucleic acid sequence (P-5).

FIG. 5 is a schematic of nucleic acids comprising a response element operably linked to a reporter nucleotide sequence and, in some instances, further comprising a second reporter nucleic acid operably linked to a constitutive promoter.

FIGS. 6A and 6B illustrate AAV9 infection (%) (FIG. 6A) or mean fluorescence intensity (FIG. 6B) (y-axis) for unmodified HEK293 cells, HEK293 cells which overexpress AAVR, unmodified Hela cells, and HeLa cells which overexpress AAVR.

FIGS. 7A and 7B illustrate AAV9 infection (%) (FIG. 7A) or mean fluorescence intensity (FIG. 7B) (y-axis) for unmodified HEK293 cells, HEK293 cells which overexpress AAVR, unmodified CHO cells, and CHO cells which overexpress AAVR.

FIGS. 8A and 8B illustrate green fluorescent protein expression (%) (FIG. 8A) or mean fluorescence intensity (FIG. 8B) (y-axis) for subclones of HEK cells modified to overexpress AAVR. All subclones tested demonstrated improved infection rates (measured as transgene expression) compared to unmodified cells.

FIGS. 9A and 9B are graphs illustrating fold change of VP64 in HEK293 cells and Hela cells modified to overexpress AAVR at various multiplicity of infections (MOI; viral genome/cell).

FIGS. 10A-10D are bar graphs illustrating AAV infection (%) for various AAV serotypes in HEK293 cells modified to overexpress AAVR (FIG. 10A) or Hela cells modified to overexpress AAVR (FIG. 10B), and mean fluorescence intensity observed from the same AAV vectors in the HEK293 cells modified to overexpress AAVR (FIG. 10C) or Hela cells modified to overexpress AAVR (FIG. 10D).

FIG. 11 illustrates AAV9 transduction in a HeRC32 cell line compared to HerRC32-AAVR clonal cell line.

FIG. 12 illustrates relative bioluminescence (RLU) in cells assayed 48 h post-transduction. Plasmid-transfected conditions or AAV9 transduced conditions at MOI (0, 10⁴, 10⁵, 10⁶) are indicated on the x-axis. Each bar represents the mean of triplicate wells, error bars indicate standard deviation.

FIG. 13 is a schematic of nucleic acids comprising a response element operably linked to a reporter nucleotide sequence and lentiviral backbones used in the study described in Example 6.

FIGS. 14A and 14B are graphs illustrating reporter expression (measured as relative light units, y-axis) in cells engineered to integrate response element-reporter constructs into the cellular genomes in response to various multiplicity of infection of AAV vectors encoding a modulator (MOI; viral genome/cell).

FIGS. 15A-15C correspond to data described in Example 6 with respect to response element-reporter construct P-6. FIGS. 15A and 15B are graphs illustrating reporter expression (measured as relative light units, y-axis) in cells engineered to integrate response element-reporter nucleic acids into the cellular genomes in response to various multiplicity of infection of AAV vectors encoding a modulator (MOI; viral genome/cell). FIG. 15A corresponds to luciferase expression mediated by the response element; FIG. 15B corresponds to firefly luciferase expression mediated by a constitutive promoter. FIG. 15C is a schematic of the P-6 construct.

FIG. 16 illustrates dose-dependent induction of luciferase reporter activity by a modulator (here SEQ ID NO: 84) in clonal cell lines comprising a response element-reporter construct stably integrated into the cellular genome and which are engineered to stably express AAVR. Relative potency measurements are presented by parallel line analysis. The full MOI range from the experiment is represented.

FIG. 17 illustrates dose-dependent induction of luciferase reporter activity by a modulator (here, SEQ ID NO: 84) in clonal cell lines comprising a response element-reporter construct stably integrated into the cellular genome and which are engineered to stably express AAVR. Relative potency measurements are presented by parallel line analysis. Whereas FIG. 16 includes the full MOI range from the experiment, the relative potency analysis for FIG. 17 was restricted to four dose points in the best linear dose range for each cell line.

FIG. 18A illustrates an eTF and control eTF DNA binding protein, Δz1 eTF, mutated in the DNA-binding alpha-helix zinc finger domains.

FIG. 18B illustrates dose response to increasing MOI in eTF-expressing (triangle, AAV9-CBA-z1-eTF) or control Δz1 eTF-expressing (square, AAV9-CBA-Δz1-eTF, SEQ ID NO: 96) AAV variants. A reference standard (RS, SEQ ID NO: 94) is shown as circles.

FIG. 19 illustrates assay specificity to eTF expressing transgenes in matched control samples. Dose response to increasing MOI in assay control (AC, red, SEQ ID NO: 94) or reference standard samples (green, CBA-Δz1-eTF, SEQ ID NO: 96). Matched control Δz1 eTF-expressing AAV sample (blue, S20-1052 (bottom line)) does not activate reporter expression.

### DETAILED DESCRIPTION

The present disclosure provides materials and methods useful for characterizing the potency of an AAV gene therapy preparation. Prior methods of estimating potency of gene therapy vectors entailed measuring viral genomes or empty viral capsids. While these methods are helpful for characterizing the amount of nucleic acid in a preparation, the methods fail to accurately inform about the biological activity associated with a preparation. Gene therapy vectors exert a therapeutic effect by delivering a payload into a cell, which then exerts a biological effect (i.e., the payload, itself, mediates a biological effect or encodes a protein or nucleic acid which mediates a biological effect). Mere quantitation of vector genomes or empty capsids does not adequately capture these features contributing to gene therapy vector activity and function, i.e., vector potency. The system and method described herein provide an accurate, elegant method for measuring potency, thus allowing more confidence in consistency between product batches and accurate patient dosing.

In one aspect, the disclosure provides a nucleic acid comprising a response element operably linked to a reporter nucleotide sequence. A response element is a nucleic acid sequence which may be recognized and bound by a transcription factor. A transcription factor is generally a protein which controls the rate of DNA transcription by binding to a specific nucleotide sequence (for example, the response element) and regulating expression by, e.g., facilitating or impeding the recruitment of RNA polymerase or other expression co-factors. Transcription factors typically contain at least one DNA binding domain, which can bind a transcription factor binding site in the target DNA, and a transcription modulation domain, which comprises binding sites for other proteins that promote or repress expression of a target nucleic acid sequence. Transcription factors may act through any one of a variety of mechanisms, including, but not limited to, stabilizing or blocking the binding of RNA polymerase to DNA, catalyzing the acetylation or deacetylation of histone proteins, recruiting coactivator or corepressor proteins to the transcription factor-DNA complex. In various aspects, the transcription factor is a transcriptional activator (i.e., it promotes transcription of a nucleic acid sequence). In alternative aspects, the transcription factor is a transcriptional repressor (i.e., it reduces or blocks transcription of a nucleic acid sequence). A transcription factor (TF) may be endogenous (i.e., one naturally expressed by a host cell) or may be exogenous (i.e., recombinantly produced in the host cell and, optionally, engineered to contain one or more modifications compared to a wild-type transcription factor). A TF may be naturally occurring, modified from a naturally occurring TF, or may be a non-naturally occurring synthetic TF.

Examples of structures of TF DNA binding domains include, but are not limited to, helix-turn-helix, zinc fingers, leucine zipper (e.g., bZIP), helix-loop-helix, and beta-scaffold. A TF may be engineered such that, e.g., a DNA binding domain is operably linked to a transcription modulation domain to which the DNA binding domain is not naturally linked (e.g., derived from a different transcription factor or from a different species). For example, a zinc finger DNA-binding domain or a transcription factor-like effector DNA-binding domain may be fused to transcription modulation domain (e.g., VP16 or VP64). Alternatively, or in addition, a TF can comprise an engineered DNA binding domain specific for a TF binding site of interest. In this regard, the TF may comprise multiple copies of the same DNA binding domain or may comprise multiple DNA binding domains of different sequences. For example, various aspects of the disclosure provide a TF with an engineered DNA binding site comprising from 2 to 10 DNA binding domains, such as zinc fingers (e.g., 3 to 8 zinc fingers, or 1, 2, 3, 4, 5, 6, 7, 8, 9, or 10 zinc fingers). Examples of engineered DNA binding domains are provided as SEQ ID NOs: 85-90. See also International Patent Publication No. WO 2020/243651, incorporated herein by reference in its entirety.

Transcription factors may be active in most cell types. Transcription factors also may be a tissue-specific, such as those from muscle cells (e.g., MyoD and muscle enhancer factor 2 (MEF2)) or those from neuronal cells (e.g., nuclear factor 1C (NF1C), nuclear factor 1X (NF1X), Brain-1 (Brn-1), or Brain-2 (Brn-2)). Transcription factors may also be ligand-dependent. Ligand-dependent transcription factors comprise an additional domain which is bound by the ligand. The activity of the ligand-dependent transcription factor may depend on whether or not it is bound to its ligand. For example, a ligand-dependent binding factor may be a transcriptional repressor in the absence of the ligand, and a transcriptional activator in the presence of the ligand. Steroid hormone receptors and nuclear receptors are examples of ligand-dependent transcription factors. Other examples of ligand-dependent transcription factors include metal-responsive transcription factors, such as those that regulate metal (iron, zinc, or copper) homeostasis. The transcription factor of the present disclosure may be a ligand-dependent transcription factor, wherein the ligand is a metal, such as iron, zinc, nickel, manganese, magnesium, potassium, sodium, molybdate, or copper. In one embodiment, the ligand of a ligand-dependent transcription factor described herein is copper. Metal-responsive transcription factors include, but are not limited to, Aft1, Aft2, Fep1, SREA, Urbs1, Ace1, Amt1, Srf1, Mac1, Cuf1, GRISEA, Crr1, Zap1, and metal response element-binding transcription factor-1 (MTF-1). MTF-1 induces expression of metallothioneins and other genes involved in metal homeostasis in response to heavy metals such as copper. MTF-1 binds to transcription factor binding sites comprising DNA sequence motifs known as metal response elements (MREs) with a core consensus TGCRCNC, wherein R is any purine (A or G) and N is any base (SEQ ID NO: 33). See, e.g., Rutherford and Bird, Eukaryot Cell. 2004 February; 3 (1): 1-13; and Wang et al., Biol Chem. 2004 July; 385 (7): 623-32.

A transcription modulation domain (TMD) is a region of a TF containing binding site for other proteins that promote or repress transcription of a target nucleic acid sequence. The TMD may contact transcriptional machinery (e.g., RNA polymerase) directly or through other proteins (known as coactivators or comodulators). The TMD(s) and DNA binding domain(s) (DBD) may be derived from different proteins. An engineered TF may comprise more than one TMD, and two or more of the TMDs may be derived from (e.g., isolated from) different proteins. In various aspects, the TMD is a transactivation domain, which enhances or upregulates expression. Examples of transactivation domains include, e.g., VP64 (SEQ ID NO: 76), VPR (SEQ ID NO: 77), VP16, VP128, p65, p300, CBP/p300-interacting transactivator 2 (CITED2) (SEQ ID NO: 78 or 79), CBP/p300-interacting transactivator 4 (CITED4) (SEQ ID NO: 80 or 81), EGR1 (SEQ ID NO: 82), or EGR3 (SEQ ID NO: 83). See also International Patent Publication No. WO 2019/109051, incorporated herein by reference in its entirety and particularly with respect to disclosure relating to transactivation domain sequences. In some aspects, the TMD is a repressive domain, which decreases or prevents expression.

A DBD and a TMD may be directly linked, e.g., with no intervening amino acid sequence. Alternatively, a DBD and a TMD may be linked via a peptide linker. In various embodiments, a DBD is conjugated to a TMD via a linker having 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, or 100 amino acids, or from 1-5, 1-10, 1-20, 1-30, 1-40, 1-50, 1-75, 1-100, 5-10, 5-20, 5-30, 5-40, 5-50, 5-75, 5-100, 10-20, 10-30, 10-40, 10-50, 10-75, 10-100, 20-30, 20-40, 20-50, 20-75, or 20-100 amino acids. In some instances, the DBD and the TMD are conjugated via naturally occurring intervening residues found in the naturally occurring proteins from which the domains are derived, or from another naturally occurring transcription factor. In other embodiments, the DBD and TMD are conjugated via a synthetic or exogenous linker sequence. Suitable linkers can be flexible, cleavable, non-cleavable, hydrophilic and/or hydrophobic. In certain embodiments, a DBD and a TMD may be fused together via a linker comprising a plurality of glycine and/or serine residues. Examples of glycine/serine peptide linkers include [GS]n, [GGGS]n (SEQ ID NO: 4), [GGGGS]n (SEQ ID NO: 5), or [GGSG]n (SEQ ID NO: 6), wherein n is an integer equal to or greater than 1. In various aspects, a linker useful for conjugating a DBD and a TAD is GGSGGGSG (SEQ ID NO: 7). In various embodiments, when a DBD is conjugated to two TMDs, the first and second TMDs may be conjugated to the DBD with the same or different linkers, or one TMD may be conjugated to the DBD with a linker and the other TMD is directly conjugated to the DBD (e.g., without an intervening linker sequence), or both TMDs may be directly conjugated to the DBD (e.g., without intervening linker sequences).

Examples of transcription factors include, but are not limited to, AF-4 transcription factors, Androgen receptor transcription factors, AP-2 transcription factors, ARID transcription factors, bHLH transcription factors, C/EBP transcription factors, CBF transcription factors, CG-1 transcription factors, COE transcription factors, COUP transcription factors, CP2 transcription factors, CSD transcription factors, CSL transcription factors, CTF/NFI transcription factors, CUT transcription factors, DM transcription factors, E2F transcription factors, EAF2 transcription factors, Ecdystd receptor transcription factors, ETS transcription factors, Fork head transcription factors, GCM transcription factors, GCR transcription factors, GTF21 transcription factors, HMG transcription factors, HMGI/HMGY transcription factors, Homeobox transcription factors, HSF transcription factors, HTH transcription factors, IRF transcription factors, MBD transcription factors, MH1 transcription factors, MYB transcription factors, NDT80/PhoG transcription factors, NF-YA transcription factors, NF-YB/C transcription factors, Nrf1 transcription factors, Nuclear orphan receptor transcription factors, Oestrogen receptor transcription factors, P53 transcription factors, PAX transcription factors, PC4 transcription factors, POU transcription factors, PPAR receptor transcription factors, PREB transcription factors, Progesterone receptor transcription factors, Prox1 transcription factors, Retinoic acid receptor transcription factors, RFX transcription factors, RHD transcription factors, ROR receptor transcription factors, Runt transcription factors, SAND transcription factors, SPZ1 transcription factors, SRF transcription factors, STAT transcription factors, T-box transcription factors, TEA transcription factors, TF-bZIP transcription factors, TF-Otx transcription factors, THAP transcription factors, Thyroid hormone receptor transcription factors, TSC22 transcription factors, Tub transcription factors, ZBTB transcription factors, zf-BED transcription factors, zf-C2H2 transcription factors, zf-C2HC transcription factors, zf-GATA transcription factors, zf-LITAF-like transcription factors, zf-MIZ transcription factors, and zf-NF-X1 transcription factors.

The nucleic acid of the disclosure comprises a response element which is recognized and bound by a transcription factor. A response element comprises one or more TF binding sites, which comprise a target nucleic acid sequence to which the DNA binding domain of the TF can bind. Binding site motifs found within the genome for many transcription factors have been identified and characterized. See, e.g., Inukai et al, Curr Opin Genet Dev. 2017 April; 43:110-119 (doi: 10.1016/j.gde.2017.02.007); Weirauch et al, “HOCOMOCO: expansion and enhancement of the collection of transcription factor binding sites models.” Cell. 2014; 158:1431-1443; Kulakovskiy et al., “JASPAR 2016: a major expansion and update of the open-access database of transcription factor binding profiles.” Nucleic Acids Res. 2016; 44:D116-125; and Wingender et al., “The TRANSFAC project as an example of framework technology that supports the analysis of genomic regulation.” Brief Bioinform. 2008; 9:326-332.

A response element of the disclosure may comprise a single TF binding site, or may comprise multiple TF binding sites. For instance, a response element may comprise 2 to 10 copies (e.g., 2, 3, 4, 5, 6, 7, 8, 9, or 10 copies) of a TF binding site. In instances where multiple TF binding sites are found in the response element, the multiple TF binding sites may all be the same (i.e., multiple copies of the same TF binding site sequence) or may be different (i.e., two or more of the TF binding sites having different nucleic acid sequences). Optionally, where multiple, different TF binding sites are present, the different TF binding sites may be recognized by different TFs or the same TF.

In various aspects, the response element comprises 2 to 10 copies (e.g., 2, 3, 4, 5, 6, 7, 8, 9, or 10 copies) of a Z1 transcription factor (TF) binding site. For example, the response element optionally comprises from 3 to 8 copies of the Z1 TF binding site. A nucleic acid sequence of a Z1 TF binding site is provided as SEQ ID NO: 1. It is understood that a TF can recognize a multitude of DNA binding site sequences. See, e.g., Siggers et al., Nucleic Acids Res. 2014; 42:2099-2111. Hence, the TF binding site may comprise 1, 2, or 3 nucleotide differences from SEQ ID NO: 1 (i.e., the TF binding site may comprise SEQ ID NO:1 having substitutions at 1, 2, or 3 nucleotide positions within SEQ ID NO: 1). Thus, in various aspects of the disclosure, each of the copies of the Z1 TF binding site has a nucleic acid sequence of SEQ ID NO: 1 or a nucleic acid sequence having 1, 2, or 3 nucleotide differences from SEQ ID NO: 1. In this respect, each of the copies may comprise the sequence of SEQ ID NO: 1, a subset of the copies may comprise SEQ ID NO: 1 and other copies may comprise SEQ ID NO: 1 comprising one more substitutions, or each of the copies may comprise SEQ ID NO:1 having 1, 2, or 3 substitutions. In some aspects, the response element may comprise a sequence having at least 80%, 85%, 90%, 95% or 98% sequence identity to SEQ ID NO. 1.

In various aspects, the response element comprises a TF binding site bound by an endogenous metal-dependent TF, such as a copper-dependent TF. For example, the response element may comprise 1 to 10 copies or 2 to 10 copies (e.g., 2, 3, 4, 5, 6, 7, 8, 9, or 10 copies) of TF binding site bound by an endogenous metal-dependent TF. In various aspects, the response element comprises one or more (e.g., 2 or more) copies of a MTF-1 TF binding site. For example, the response element optionally comprises from 3 to 8 copies of the MTF-1 TF binding site. A nucleic acid sequence of a MTF-1 TF binding site is provided as SEQ ID NO: 8. The TF binding site may comprise 1, 2, or 3 nucleotide differences from SEQ ID NO: 8 (i.e., the TF binding site may comprise SEQ ID NO: 8 having substitutions at 1, 2, or 3 nucleotide positions within SEQ ID NO: 8). Thus, in various aspects of the disclosure, each of the copies of the MTF-1 TF binding site has a nucleic acid sequence of SEQ ID NO: 8 or a nucleic acid sequence having 1, 2, or 3 nucleotide differences from SEQ ID NO: 8. In this respect, each of the copies may comprise the sequence of SEQ ID NO: 8, a subset of the copies may comprise SEQ ID NO: 8 and other copies may comprise SEQ ID NO: 8 comprising one more substitutions, or each of the copies may comprise SEQ ID NO: 8 having 1, 2, or 3 substitutions. In some aspects, the response element may comprise a sequence having at least 80%, 85%, 90%, 95% or 98% sequence identity (e.g., 100% identity) to SEQ ID NO: 8.

If desired, a response element comprising multiple TF binding sites may comprise a spacer sequence between two or more of the TF binding sites. The spacer may be of any length so long as the TF binding and modulation of gene expression is not abrogated. In some embodiments, the response element comprises between 2 and 10 copies of a Z1 TF binding site and further comprises a spacer sequence between at least two of the Z1 TF binding site copies (optionally between each of the copies of the Z1 TF binding site). Examples of spacer sequences include the nucleic acid sequence of SEQ ID NO: 2 (CTCGAT) and SEQ ID NO: 3 (GATAGGGAGTAAACTCGA). In some aspects, the spacer sequence may comprise a sequence having at least 80%, 85%, 90%, 95% or 98% sequence identity to SEQ ID NO: 2 or 3.

In various aspects of the disclosure, the response element-comprising nucleic acid comprises a sequence of any one of SEQ ID NOs: 10-15, SEQ ID NOs: 17-23, and SEQ ID NO: 25. The disclosure further contemplates a nucleic acid comprising 1, 2, or 3 nucleotide differences with respect to a sequence of any one of SEQ ID NOs: 10-15, SEQ ID NOs: 17-23, and SEQ ID NO: 25. In some aspects, the response element comprising nucleic acid may comprise a sequence having at least 80%, 85%, 90%, 95% or 98% sequence identity to any one of SEQ ID NOs: 10-15, SEQ ID NOs: 17-23, and SEQ ID NO: 25.

Optionally, the response element further comprises a promoter which further drives expression in a host cell. In various aspects, the response element comprises two promoters.

A promoter can be native or non-native to the nucleic acid sequence to which it is operably linked, and native or non-native to a particular host cell. A promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. An example of a tissue-specific promoter is a liver-specific promoter, such as an HLP promoter. Additional examples of promoters include, but are not limited to, LPL, HCR-hAAT, ApoE-hAAT, and LSP promoters. These promoters are described in more detail in the following references: HLP: Mcintosh J, et al, Blood 2013 Apr. 25, 121 (17): 3335-44; LPL: Nathwani et al, Blood. 2006 Apr. 1, 107(7): 2653-2661; HCR-hAAT: Miao et al, Mol Ther. 2000; 1:522-532; ApoE-hAAT: Okuyama et al, Human Gene Therapy, 7, 637-645 (1996); and LSP: Wang et al, Proc Natl Acad Sci USA. 1999 Mar. 30, 96 (7): 3906-3910.

In some aspects, the promoter is a minimal promoter. Minimal promoters are those which typically cannot drive expression without the presence of additional regulatory elements. An example of a minimal promoter suitable for use in the context of the disclosure is minP (SEQ ID NO: 32). Other minimal promoters include, but are not limited to, the CMV-minimal promoter, hsp70 minimal promoter, the minimal promoter included in the tetracycline response element, and the MinTk minimal promoter.

The nucleic acid of the disclosure comprises a response element operably linked to a reporter nucleotide sequence. “Operably linked” refers to functional linkage between nucleic acid sequences or amino acid sequences. For example, “operably linked” means that a control sequence is in a correct location and orientation in relation to another nucleic acid sequence to exert its effect (e.g., initiation of transcription) on the nucleic acid sequence. Here, a response element is operably linked to the reporter nucleotide sequence, such that the response element is capable of modulating the rate of expression or the abundance of the encoded reporter. Generally, operably linked DNA sequences are contiguous and, where necessary, in the same reading frame (although this is not always required).

A “reporter” refers to any biomolecule (e.g., protein) which directly or indirectly creates a detectable signal. In various aspects, the reporter nucleotide sequence encodes a luminescent protein or an enzyme that produces bioluminescence. Examples of reporters include, but are not limited to, green fluorescent protein (GFP), variant of green fluorescent protein (GFP10), enhanced GFP (eGFP), TurboGFP, GFPS65T, TagGFP2, mUKGEmerald GFP, Superfolder GFP, GFPuv, destabilised EGFP (dEGFP), Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, destabilised ECFP (dECFP), CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed, t-HcRed, HcRed-Tandem, mPlum, mNeptune, NirFP, Kindling, far red fluorescent protein, yellow fluorescent protein (YFP), eYFP, destabilised EYFP (dEYFP), TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKOK, mKO2, mGrapel, mGrape2, zsYellow, eqFP611, Sirius, Sandercyanin, shBFP-N158S/L1731, near infrared proteins, iFP1.4, iRFP713, iRFP670, iRFP682, iRFP702, iRFP720, IFP2.0, mIFP, TDsmURFP, miRFP670, Brilliant Violet (BV) 421, BV 605, BV 510, BV 711, BV786, PerCP, PerCP/Cy5.5, DsRed, DsRed2, mRFP1, pocilloporin, Renilla GFP, Monster GFP, paGFP, or a Phycobiliprotein, luciferase, LacZ, alkaline phosphatase, secreted embryonic alkaline phosphatase (SEAP), chloramphenicol acetyl transferase (CAT), beta-galactosidase, and beta-glucuronidase (GUS), as well as biologically active variants and fragments of the foregoing. In various aspects, the reporter nucleic acid encodes GFP, EGFP, mCherry, or luciferase.

If desired, the nucleic acid may comprise a second reporter nucleotide sequence which is not operably linked to the response element. Any reporter, including any of the reporters described herein, is suitable for use as a second reporter. Examples of second reporter nucleotide sequences include, but are not limited to, nucleotide sequences that encode a luminescent protein (e.g., green fluorescent protein (GFP), enhanced GFP (EGFP), or mCherry) or encode an enzyme that produces bioluminescence (e.g., a luciferase). A second reporter may be chosen to have a signal which is complementary to a first reporter. As an example, if the first reporter is a fluorescent reporter and the second reporter may be a fluorescent reporter with a different excitation and/or emission wavelengths. In another example, if the first reporter is luciferase the second reporter may be Renilla, or if the first reporter is an alkaline phosphatase the second reporter may be LacZ. The second reporter nucleic acid is, in various aspects, operably linked to a separate promoter (i.e., a promoter which is different than the promoter that is part of the response element, if the response element comprises a promoter). Suitable promoters include, but are not limited to, the promoters described herein. Optionally, the separate promoter is a constitutive promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters.

In various aspects, the nucleic acid provided herein comprises one more additional regulatory elements (optionally in addition to a promoter), such as, for example, sequences associated with transcription initiation or termination, enhancer sequences, and efficient RNA processing signals. Exemplary regulatory elements include, for example, an intron, an enhancer, UTR, stability element, WPRE sequence, a Kozak consensus sequence, posttranslational response element, a microRNA binding site, a polyadenylation (polyA) signal sequence, or a combination thereof. Regulatory elements can function to modulate gene expression at the transcriptional phase, post-transcriptional phase, or at the translational phase of gene expression. At the RNA level, regulation can occur at the level of translation (e.g., stability elements that stabilize mRNA for translation), RNA cleavage, RNA splicing, and/or transcriptional termination.

In certain embodiments, the nucleic acid further comprises a polyA signal sequence. Suitable polyA signal sequences include, for example, an artificial polyA that is about 75 bp in length (PA75) (see e.g., International Patent Publication No. WO 2018/126116), the bovine growth hormone polyA, SV40 early polyA signal, SV40 late polyA signal, rabbit beta globin polyA, HSV thymidine kinase polyA, protamine gene polyA, adenovirus 5 EIb polyA, growth hormone polyA, or a PBGD polyA. In exemplary aspects, a polyA sequence suitable for use in the expression cassettes provided herein is an hGH polyA (SEQ ID NO: 27) or a synthetic polyA (SEQ ID NO: 28 or SEQ ID NO: 91). In various aspects, the polyA comprises the nucleic acid sequence of SEQ ID NO: 91. Typically, the polyA signal sequence is operably linked to the reporter nucleic acid sequence.

The disclosure further provides a cell comprising the nucleic acid described herein. The nucleic acid may be stably integrated into the genome of the cell or may be present in a separate expression vector construct. The cell can be a cell from any organism (e.g., a prokaryotic cell, a eukaryotic cell, a bacterial cell, a plant cell, an algal cell, a fungal cell (e.g., a yeast cell), a mammalian cell, an animal cell (human or non-human), etc.). Mammalian cells include those isolated or derived from, e.g., humans, non-human primates (such as apes, chimpanzees, monkeys, and orangutans), domesticated animals (including dogs and cats), livestock (such as horses, cattle, pigs, sheep, and goats), or other mammalian species including, without limitation, mice, rats, guinea pigs, rabbits, hamsters, and the like. The cells also may be isolated or derived from any tissue. In various aspects, the cells are central nervous system cells, frontal cortex cells, glial cells, microglial cells, or striatum cells. Examples of cells include, but are not limited to, Chinese Hamster Ovary (CHO) cells and derivatives thereof (e.g., CHO-K1, CHO pro-3), mouse myeloma cells (e.g., NS0, GS-NS0, Sp2/0), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa and derivatives such as HeRC32), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human neuroblastoma cells SH-SY5Y, human liver cancer cells Hep G2, mouse B myeloma cells J558L, and baby hamster kidney (BHK) cells (Gaillet et al. 2007; Khan, Adv Pharm Bull 3 (2): 257-263 (2013)).

In some aspects, the cell has been modified with an exogenous nucleic acid comprising a nucleotide sequence encoding a receptor that improves transduction efficiency of an expression vector of interest. In this regard, the disclosure provides a cell that has been modified with an exogenous nucleic acid encoding an adeno-associated virus receptor (AAVR). The cell is optionally engineered to stably overexpress the AAVR. By “overexpress” is meant increasing the overall amount of AAVR in the cell (i.e., the cell produces more of an AAVR than a matched cell which has not been modified). The cell may, or may not, naturally express AAVR prior to the modification. The AAVR may be a wild-type AAVR or a modified AAVR. Expression of the AAVR enhances AAV infection of a host cell by, e.g., increasing the number of receptors on the cell surface, presenting AAVR with enhanced affinity to AAV coat proteins, or facilitating entry of AVV into the cell.

Wild type AAVR is a predicted type I transmembrane protein. The protein includes a signal peptide, a MANSC domain (motif at N terminus with seven cysteines), and five Ig-like domains (polycystic kidney disease (PKD) domains 1-5). A transmembrane domain is located C-terminal to the MANSC and PKD domains, and is followed by a cytoplasmic tail. The structure of AAVR is further characterized in, e.g., Summerford et al., Molecular Therapy, 24 (4): 663 (2016); Meyer et al., eLife 8: e44707 (2019); and International Patent Publication No. WO 2017/083423 (incorporated here by reference in their entireties and particularly with respect to disclosure of AAVR structure, AAVR sequences, and variant AAVRs). The AAVR may be from any species, e.g., a mammalian AAVR protein, such as a rodent AAVR protein, a primate AAVR protein, a rat AAVR protein, a mouse AAVR protein, a pig AAVR protein, a cow AAVR protein, a sheep AAVR protein, a rabbit AAVR protein, a dog AAVR protein, or a human AAVR protein. Preferably, the AAVR is a human AAVR. A wild type human AAVR amino acid sequence is provided herein as SEQ ID NO: 92. The AAVR may also be a variant AAVR, such as any of the variant AAVRs described in International Patent Publication No. WO 2017/083423, hereby incorporated by reference.

Thus, the disclosure provides a cell comprising a nucleic acid comprising a response element as described herein operably linked to a reporter nucleotide sequence, wherein the cell is engineered to stably overexpress an AAVR, such as a wild-type AAVR. The response element may comprise from 2 to 10 copies of a transcription factor (TF) binding site (e.g., from 3 to 8 copies or from 3 to 6 copies of the TF binding site), although one copy of the TF binding site also is contemplated. The TF binding site may comprise a sequence bound by an endogenous TF or an exogenous TF, as described above. In various aspects, the TF binding site is bound by a ligand-dependent TF, such as a metal-responsive TF (e.g., a copper-responsive TF, such as MTF-1, which recognizes a binding comprising SEQ ID NO:8). In various aspects, the TF binding site comprises a sequence bound by an exogenous TF, such as an engineered TF comprising a modified DNA binding site having from 2 to 10 zinc fingers. In various aspects, the TF binding site comprises one or more Z1 TF binding sites. In this respect, the TF binding site comprises, in various embodiments, SEQ ID NO: 1. Alternatively or in addition, the TF binding site is optionally recognized by an engineered DNA binding domain comprising a sequence selected from group consisting of SEQ ID NOs: 85-90 and/or a peptide comprising one or more of the DNA binding domains (such as a peptide comprising the amino acid sequence of SEQ ID NO: 93).

In various aspects, the disclosure provides a composition comprising a cell comprising a response element (optionally engineered to stably express AAVR) and a TF that binds the response element. In this respect, the cell may comprise any one or more of the TFs described herein. The cell may naturally produce the TF (i.e., the TF is endogenous), or the TF may be exogenous with respect to the host cell (i.e., expressed from an exogenous nucleic acid introduced into the cell). In various aspects, the TF is a transcriptional activator, although transcriptional repressors are also envisioned. As described above, a representative transcription factor binds to the Z1 TF binding site. In this regard, the TF may comprise an engineered Z1 binding domain, such as a Z1 binding domain comprising SEQ ID NO: 1. Alternatively, the TF binds the TF binding site recognized by MTF-1. The TF may be a metal-responsive TF (e.g., a copper-responsive TF, such as MTF-1). Optionally, the TF is MTF-1 or comprises the DBD of MTF-1.

Any of the nucleic acids described herein, including the nucleic acid comprising the response element operably linked to a reporter nucleic acid, a nucleic acid encoding a TF, and the like, may be provided in an expression vector. An “expression vector” is any molecule or moiety which transports, transduces, or otherwise acts as a carrier of a heterologous polynucleotide(s). A vector may be an integrating or non-integrating vector, referring to the ability of the vector to integrate a nucleic acid into the genome of the host cell. Examples of expression vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; and transposons (e.g., PiggyBac); and (b) viral vectors such as retroviral vectors, lentiviral vectors, adenoviral vectors, and adeno-associated viral vectors. In various embodiments, the expression vector is a viral vector. Viral vectors can be obtained by deleting all, or some, of the coding regions from the viral genome, but leaving intact those sequences (e.g., terminal repeat sequences) that may be necessary or advantageous for functions such as packaging the vector genome into the virus capsid.

Viral vectors of the present invention may be produced recombinantly and may be based on adeno-associated virus (AAV) parent or reference sequence. AAV is a small, replication-defective, non-enveloped animal virus that infects humans and some other primate species. AAV vectors can also infect both dividing and quiescent cells without integrating into the host cell genome. The AAV genome consists of a linear single stranded DNA which is ~4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion. In an exemplary aspect, an AAV vector provided herein does not contain the rep or cap genes. It will be appreciated that AAV-based vectors are typically packaged into viral particles able to infect host cells. As such, “AAV vector” as used herein encompasses AAV viral particles comprising at least one AAV capsid protein and an encapsidated AAV polynucleotide comprising a transgene.

Serotypes which may be useful in the context of the disclosure include any of those arising from AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrhIO, AAV-DJ, and AAV-DJ8. Serotypes generally differ in their tropism, or the types of cells they infect. AAVs may comprise the genome and capsids from multiple serotypes (e.g., pseudotypes). For example, an AAV may comprise the genome of serotype 2 (e.g., ITRs) packaged in the capsid from serotype 5 or serotype 9. Pseudotyped vectors may demonstrate improved transduction efficiency as well as altered tropism. In some cases, an AAV serotype that can cross the blood brain barrier or infect cells of the CNS is preferred. The AAV can be a self-complementary AAV (scAAV). See, e.g., Raj et al., Expert Rev Hematol. 2011 October; 4 (5): 539-549. In certain aspects, the expression vector is an AAV vector comprising a 5′ ITR and a 3′ ITR. In some aspects, the expression vector is an AAV vector comprising a 5′ ITR, a promoter, a nucleic acid encoding a modulator (such as a TF), and a 3′ ITR. In some aspects, the expression vector is an AAV vector comprising a 5′ ITR, an enhancer, a promoter, a nucleic acid encoding a modulator (such as a TF), a polyA sequence, and a 3′ ITR. In some aspects, the AAV vector comprises the nucleic acid comprising the response element operably linked to the reporter nucleic acid. In various aspects, the AAV vector is an AAV9 vector or an scAAV9 vector. In various aspects of the disclosure, the AAV vector is an AAV3 vector. In various aspects of the disclosure, the AAV vector is an AAV8 vector.

In some aspects, the disclosure provides composition comprising (i) a cell comprising a response element, wherein the cell is optionally engineered to stably express AAVR), and (ii) a sample comprising an AAV. In an example, the composition comprises (i) a HeRC32 cell comprising a response element, wherein the response element comprises a TF binding sequence that comprises SEQ ID NO: 1 and a reporter, and (ii) a sample comprising an AAV which comprises a nucleic acid sequence which encodes a TF which binds a sequence of SEQ ID NO: 1. In some examples, the HeRC32 stably expresses an AAVR of SEQ ID NO: 92. In some examples, the response element comprises a sequence of any one of SEQ ID NOs: 10-15 and SEQ ID NOs: 17-23. In some cases, the sample comprises an AAV which comprises a nucleic acid sequence of SEQ ID NO: 94.

The disclosure further provides a method of determining the potency of a sample comprising an AAV vector. AAV vectors are described above. The method comprises contacting a cell described herein with all or part of the sample. The cell comprises a response element operably linked to a reporter nucleotide sequence, and is engineered to stably overexpress an AAVR. The AAV vector encodes a modulator that directly or indirectly modulates expression of the reporter nucleotide sequence via the response element. The method further comprises measuring expression of the reporter nucleotide sequence in the cell. In various aspects, the method then comprises determining the potency of the sample based on the measured expression level of the reporter nucleic acid sequence.

The “sample” comprising the AAV vector of interest may be any type of sample suitable for characterization of potency, typically a quantity of AAV vector in a preparation suitable for vector manufacturing, storage, or administration to patients. A sample may contain any amount of AAV vector suitable for transducing the cells to obtain a detectable signal from the reporter. For example, a sample may comprise at least about 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ AAV vectors per mL of composition. The sample is, in various aspects, a composition of AAV vectors collected at one or more points in a vector manufacturing or purification process. The sample may be a composition of AAV vectors taken from a product batch prior to shipment. The method of the disclosure may comprise other additional steps, which may further increase the purity of the AAV and remove other unwanted components and/or concentrate the fraction for testing. The method of the disclosure is suitable for, e.g., confirming the safety of an AAV vector product lot, characterizing a dose of AAV vector, evaluating activity of a vector composition, evaluating stability of a vector composition (where the method is, for example, performed at different points in time on the same sample), to show comparability of manufacturing changes, and/or determining consistency between AAV product samples.

The AAV vector encodes a modulator that directly or indirectly modulates expression of the reporter nucleotide sequence via the response element. In various aspects, the modulator comprises a DNA binding domain (DBD) that binds the TF binding domain(s) in the response element and activates or inhibits expression of the reporter nucleotide sequence, either by virtue of the binding of the response element, itself, or recruitment of other proteins that impact expression. In some embodiments, the modulator is a transcription factor that binds to TF binding sites in the response element. Transcription factors are further described above. In an exemplary embodiment, the TF is an engineered TF comprising zinc finger domains which bind Z1 binding domains operably linked to a VP64 TMD.

The transcription factor may be any of the transcription factors disclosed herein, or may comprise components of any of the referenced transcription factors referenced herein (e.g., the DNA binding domain or the transcription modulation domain of the referenced transcription factors). In exemplary aspects of the disclosure, the heterologous nucleic acid encodes a transcription factor that upregulates SCN1A production and is any of the engineered transcription factors described in International Patent Publication No. WO 2020/243651, incorporated herein by reference in its entirety. For example, in an exemplary aspect of the disclosure, the engineered transcription factor comprises a DNA binding domain comprising a zinc finger motif having the following structure: LEPGEKP-[YKCPECGKSFS X HQRTH TGEKP]n-YKCPECGKSFS X HQRTH-TGKKTS (SEQ ID NO: 29), wherein n is an integer from 1-15, e.g., 1, 2, 3, 4, 5, 6, 7, 8, 9, 10, 11, 12, 13, 14, or 15, and each X independently is a recognition sequence (e.g., a recognition helix) capable of binding to three bp of a target sequence. In exemplary embodiments, n is 3, 6 or 9. In a particularly preferred embodiment, n is 6. In various embodiments, each X may independently have the same amino acid sequence or a different amino acid sequence as compared to other X sequences in the DNA binding domain. In an exemplary embodiment, each X is a sequence comprising seven amino acids that has been designed to interact with three bp of the target binding site of interest using the Zinger Finger Design Tool from Scripps located on world wide web at scripps.edu/barbas/zfdesign/zfdesignhome.php. The engineered transcription factor optionally further comprises a VP64 transcription modulation domain. In some instances, the transcription factor may have a sequence at least 80%, at least 85%, at least 90%, at least 95%, at least 96%, at least 97%, at least 98%, or at least 99% sequence identity to any of SEQ ID NOs: 35-75.

In alternative aspects of the disclosure, the modulator is a protein that regulates metal metabolism, and the cell comprises a metal-responsive TF that binds the response element. Exemplary metal-responsive transcription factors include, e.g., Aft1, Aft2, Fep1, SREA, Urbs1, Ace1, Amt1, Srf1, Mac1, Cuf1, GRISEA, Crr1, Zap1, and MTF-1. The metal-responsive transcription factor may be endogenous to the cell or may be recombinantly made by the cell. In various aspects, the modulator is ATP7B, which is a copper-transporting P-type ATPase. The ATP7B protein is located in the trans-Golgi network of the liver and brain and balances the copper level in the body by excreting excess copper into bile and plasma. An amino acid sequence of ATP7B is provided as SEQ ID NO: 30. When expressed, ATP7B modulates the level of copper in the cell, thereby indirectly modulating reporter gene expression via the metal-responsive TF. When copper levels are high, the TF (e.g., MTF-1) drives expression of the reporter by binding the response element (e.g., a response element comprising one or more MTF-1 binding sites). When copper levels are lower, reporter expression also decreases. While the description above describes the method in the context of copper metabolism, it will be appreciated that the method is not so limited; the method may be used in connection with any ligand-dependent TF (including other metal-responsive TFs) and other modulators which mediate the amount of ligand in the cell.

Reporter nucleic acids and their encoded proteins are described above. Methods of measuring expression of the reporter nucleotide sequence in a cell are well known in the art. The amount of reporter RNA may be determined, or the amount of resulting protein may be determined. Generally, reporter expression is characterized by measuring activity or a characteristic of the protein encoded by the reporter nucleotide sequence, i.e., measuring luminescence of a luminescent protein or measuring luminescence mediated by an enzyme that produces bioluminescence. Luminescence activity can be determined by any suitable method, such as the method described in, e.g., Inouye, S. & Shimomura, O. (1977) Biochem. Biophys. Res. Commun. 233, 349-353. Indeed, fluorescence may be detected and quantified using, e.g., flow cytometry, and/or reporter mRNA levels may be quantified via RT-qPCR. The signal(s) generated by the reporter or the amount of expression product can be used to derive quantifiable absolute or relative data regarding the potency of the vector preparation tested.

In various aspects, the method comprises determining the potency of the sample based on the measured expression level of the reporter nucleic acid sequence or the expression product. In this respect, the method optionally comprises comparing the measured expression level to a standard potency curve for the AAV vector. A standard curve is generated by assessing the reporter signal for specific amounts of AAV vector (i.e., different dilutions of vector concentrations) with multiple replicates. Alternatively, a standard curve may be generated by assessing the measured signal for specific amounts of reporter nucleic acid sequence DNA, mRNA or expression product.

The disclosure further provides a system or kit comprising any combination of the components described herein. In exemplary embodiments, the system or kit comprises one, two or all of (A) a presently disclosed nucleic acid comprising a response element operably linked to a reporter nucleic acid sequence, (B) a cell which overexpresses AAVR, and/or (C) a modulator that directly or indirectly affects reporter expression via the response element. As described herein, the modulator may directly contact the response element to activate or repress transcription of the reporter nucleic acid. Alternatively, the modulator may indirectly affect transcription of the reporter nucleic acid via the response element by, e.g., modulating the amount of a ligand within the cell, thereby modulating the activity of a ligand-dependent transcription factor that binds the response element of (A). The modulator may comprise a protein or a nucleic acid comprising a sequence that encodes a protein, for instance. Thus, the disclosure provides a kit comprising a cell which overexpresses AAVR as described herein and the nucleic acid comprising a response element operably linked to a reporter nucleotide sequence, wherein the response element comprises from 2 to 10 copies of a TF binding site (e.g., a Z1 TF binding site). A kit can contain instructions for use of the components in a method, e.g., a method in accordance with the present disclosure. Ancillary materials to assist in or to enable performing such a method may be included within a kit of the disclosure.

### EXAMPLES

The following examples are given merely to illustrate the present invention and not in any way to limit its scope.

### Example 1

This example describes the construction and testing of different nucleic acid constructs comprising a response element operably linked to reporter nucleic acid.

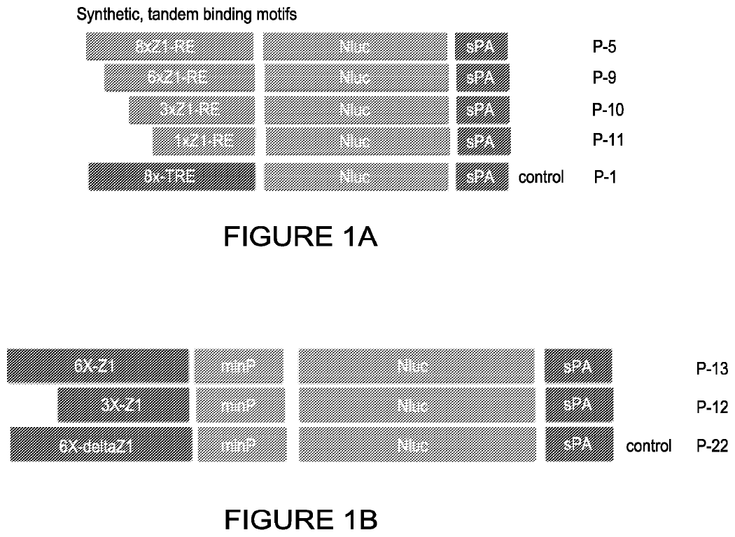

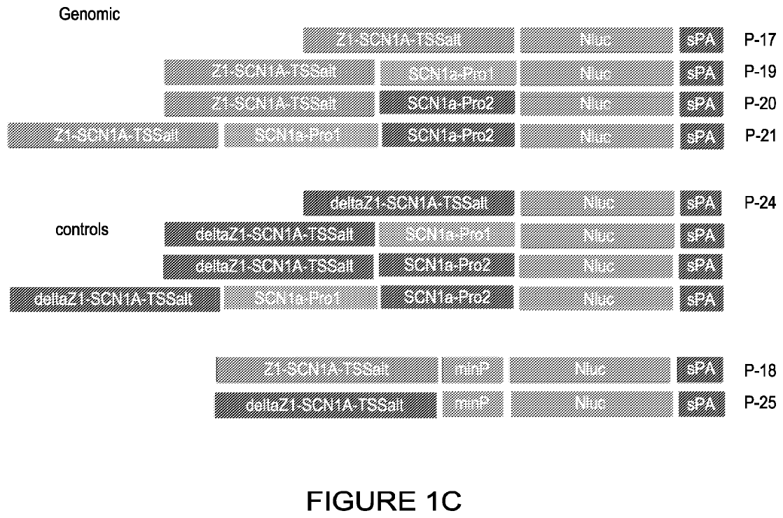

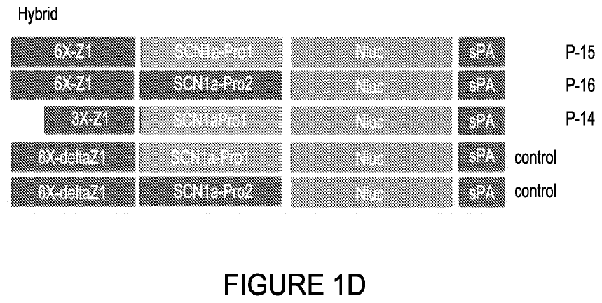

Several reporter constructs encoding N-luciferase (“Nluc”) operably linked to different response elements were constructed using standard molecular biology techniques. A summary of the components of each reporter construct is provided in Table 1 and the structures of the different reporter constructs are shown in FIG. 1. The descriptions of the response elements (RE) include the number of repeats (e.g., “8×” is eight copies) of a particular TF binding domain (e.g., the Z1 TF binding domain).

FIG. 1A details the concatenated response element design found in Class I Synthetic reporter constructs. The Z1 response element is activated by VP64 and/or CITED4 binding found in Class I Synthetic reporter constructs. Construct P1 comprises a Tet-responsive promoter PTight, consisting of seven tet operator sequences followed by the minimal CMV promoter. A second class of response elements (Class II) was comprised of multiple Z1 sequences inserted into a Tetracycline-responsive element (TRE) scaffold. FIG. 1B details the response element: minPro design found in some of the Class II Synthetic reporter constructs, namely, P13, P12 and P22. FIG. 1C details the response element: endogenous SCN1A target/promoter domains found in the Genomic reporter constructs. FIG. 1D details the hybrid design found in some reporter constructs, namely P15, P16, and P14.

TABLE 1

| | | | | Response |
|---|---|---|---|---|
| | | Response | | Element |
| Class | Construct Name | Element | Promoter | SEQ ID NO: |
| Class I Synthetic | P1 (control) | 8x TRE | TRE | 9 |
| Class I Synthetic | P5 | 7xZ1-RE | Substituted TRE | 10 |
| Class I Synthetic | P9 | 5xZ1-RE | Substituted TRE | 11 |
| Class I Synthetic | P10 | 3xZ1-RE | Substituted TRE | 12 |
| Class I Synthetic | P11 | 1xZ1-RE | Substituted TRE | 13 |
| Class II Synthetic | P13 | 6X-Z1 | minP | 14 |
| Class II Synthetic | P12 | 3X-Z1 | minP | 15 |
| Class II Synthetic | P22 (control) | 6X-deltaZ1 | minP | 16 |
| Hybrid | P15 | 6X-Z1 | SCN1a-Pro1 | 17 |
| Hybrid | P16 | 6X-Z1 | SCN1a-Pro2 | 18 |
| Hybrid | P14 | 3X-Z1 | SCN1a-Pro1 | 19 |
| Genomic | P17 | Z1-SCN1A-TS- | | 20 |
| | | Salt | | |
| Genomic | P19 | Z1-SCN1A-TS- | SCN1a-Pro1 | 21 |
| | | Salt | | |
| Genomic | P20 | Z1-SCN1A-TS- | SCN1a-Pro2 | 22 |
| | | Salt | | |
| Genomic | P21 | Z1-SCN1A-TS- | SCN1a- | 23 |
| | | Salt | Pro1 + Pro2 | |
| Genomic | P24 | deltaZ1-SCN1A- | | 24 |
| | | TS-Salt | | |
| Genomic | P18 | Z1-SCN1A-TS- | minP | 25 |
| | | Salt | | |
| | P25 | deltaZ1-SCN1A- | minP | 26 |
| | | TS-Salt | | |

The function of each reporter construct was tested as follows: HEK293T cells were placed into an individual well of a 96-well plate at approximately 10,000 cells per well. Using FuGENE® HD Transfection Reagent (Promega, Madison, WI), the cells of each well were transfected with three plasmids, the total amount of which was 100 ng. Each well of cells was triply transfected with (a) a constitutive transfection control plasmid (20 ng), (b) a reporter plasmid described in Table 1 or shown in FIG. 1 (40 ng), and (c) an activator plasmid or control filler plasmid (40 ng) (total 100 ng plasmid). The (a) constitutive transfection control plasmid contained a plasmid expressing Firefly luciferase under the control of the Herpes Simplex Virus (HSV) thymidine kinase (TK) promoter element (TK-Firefly). The (b) reporter construct contained one of the Z1-based response elements described in Table 1 and FIG. 1, or lacked a Z1 response element (designated with “delta” in the name of the response element in Table 1). If present, the Z1 TF binding domain (SEQ ID NO: 1) was present in 1-8 tandem copies (represented by “1×”, “3×”, “6×”, “8×”). The (c) activator plasmid encoded an artificial transcription factor activator which binds to the Z1 TF binding domain of the reporter construct (amino acid sequence of SEQ ID NO: 84). The control filler plasmid did not contain the artificial transcription factor activator but encoded a non-bioluminescent enhanced Green Fluorescent Protein (EGFP). Each well was expected to exhibit a baseline fluorescent signal attributed to the constitutive expression of the Firefly luciferase of this transfection control plasmid, if the cells were successfully transfected. Any fluorescent signal that was above baseline was attributed to the N-luciferase of the reporter construct. The additional luciferase signal was expected only when the reporter construct contained a Z1 response element and an activator plasmid was co-transfected into the cells. If the reporter construct did not contain a Z1 response element (those with “deltaZ1”), or, if a control filler plasmid (which did not encode transcription factor activator) was co-transfected, the cells were not expected to exhibit a fluorescent signal above baseline.

Following transfection (24 hours post-transfection), plates were lysed and diluted 1:100 in ONE-Glo EX Luciferase Assay buffer (Promega). Diluted lysate was assayed for Firefly and NanoLuc luminescence according to manufacturer's protocols (Nano-Glo Dual Luciferase Reporter Assay system, Promega), and relative light units (RLU) were measured on a bioluminescent plate reader (GloMax, Promega). Each transfection was performed in triplicate, and data reflect the average of triplicate wells (Mean±Standard Deviation).

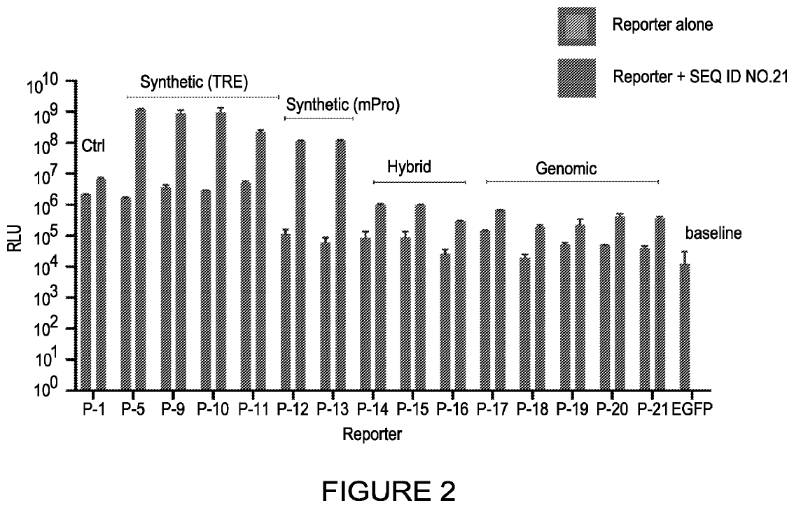

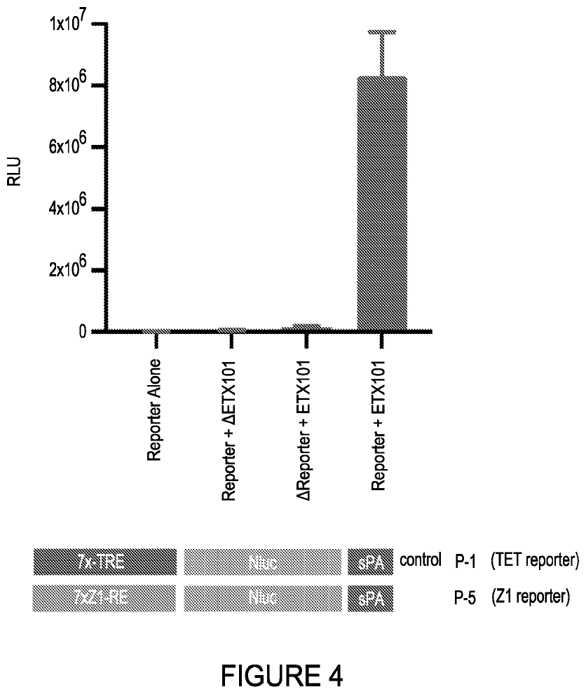

The results are shown in FIG. 2. All cells exhibited a similar baseline fluorescent signal attributed to the expression of the Firefly luciferase gene (left bars for each construct in FIG. 2), demonstrating successful transfection of the cells. When the cells were co-transfected with a reporter construct containing a Z1-based response element and an activator plasmid, a fluorescent signal above baseline was observed (right bars for reach construct in FIG. 2). The strongest Nluc reporter induction was observed in the Class I and Class II synthetic variants, with genomic and hybrid elements showing less upregulation from activator co-transfection (FIG. 4). Higher baseline and induced activity were generally observed in the Class I synthetic variants compared to the Class II variants. Based on this initial screen, a subset of synthetic variants was selected for further screening: P-5, P-9, P-10, P-11, P-12, and P-13.

These data demonstrated activator-dependent luciferase reporter induction of reporter constructs containing Z1 response elements in a plasmid transfection system.

### Example 2

This example demonstrates dose-dependent luciferase reporter induction in nucleic acids comprising a response element operably linked to a reporter nucleic acid, in a transient co-transfection system.

The lead reporter constructs from Example 1 were selected for further study. In one experiment, the sensitivity of the Class I and Class II synthetic reporter constructs to the amount of activator plasmid was assayed. Briefly, HEK293T cells were aliquoted into wells of a 96-well plate and subsequently transfected with three types of plasmids as described in Example 1. Unlike Example 1, however, different amounts of activator plasmid were used. The cells of each well received 20 ng of the constitutive transfection control plasmid, 10 ng of a reporter construct, and 70 ng total of a combination of activator plasmid and control filler plasmid. To assess the sensitivity of the reporter construct to different amounts of the activator plasmid, varied amounts of activator plasmid (0, 0.01 ng, 0.1 ng, 1 ng, 10 ng, 30 ng, 50 ng, or 70 ng) were titrated into samples containing filler plasmid so that a total of 70 ng activator and filler plasmid were used in the transfection. The cells were processed after transfection and the luminescence levels were measured as described in Example 1. Each transfection was performed in triplicate; data reflect the average of triplicate wells (Mean±Standard Deviation).

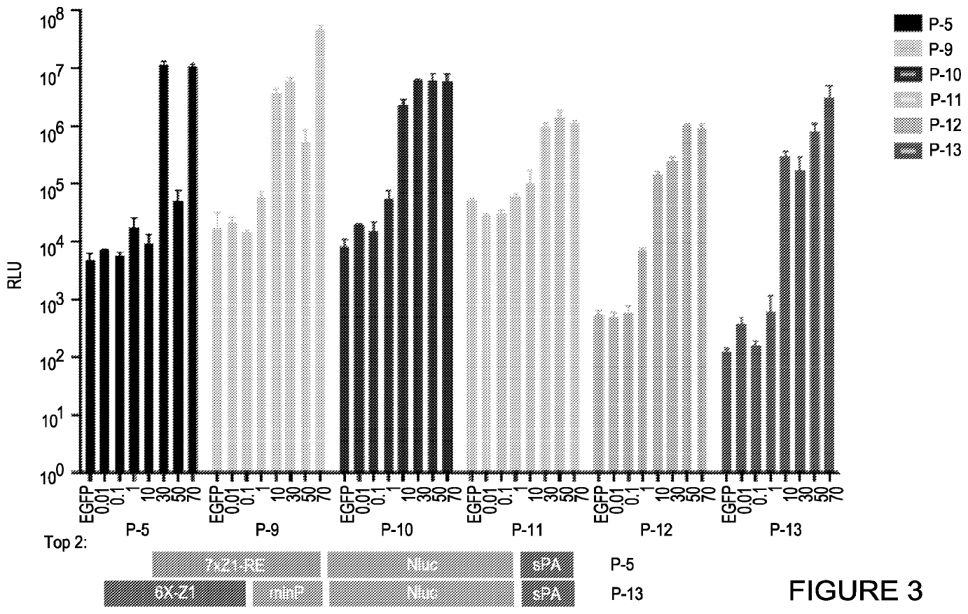

The results are shown in FIG. 3. Each reporter construct demonstrated a sensitivity to varied amounts of activator, showing a dose response result with higher activity in response to higher amounts of activator plasmid. Similar to previous observations, Class I response elements produced a stronger expression level than Class II elements, and a broader range in signal induction with increasing copies of the z1 element was also observed. Therefore, lead candidate reporter variants with the greatest number of z1 element copies were selected from each response element class, P-5 and P-13.

### Example 3

This example demonstrates that induction of reporter expression in the nucleic acid of the disclosure requires binding of the response element by the activator in the transient co-transfection system.

HEK293T cells were cultured by standard methods, and each well of a 96-well plate was triply transfected (FugeneHD) with (totaling 100 ng plasmid): a constitutive transfection control plasmid (20 ng), reporter plasmid (40 ng), and activator plasmid (40 ng). The constitutive transfection control was a plasmid expressing Firefly luciferase under the control of the HSV thymidine kinase (TK) promoter element (TK-Firefly). A control reporter plasmid included tetracycline response element (TRE) upstream of the Nluc open reading frame (“AReporter”). A Z1-based reporter plasmid contained a Z1-Response Element (7×Z1-TRE, P-5), upstream of the Nluc open reading frame. In this element, multiple Z1 sequences were inserted into a Tetracycline-responsive element (TRE) scaffold (Gossen and Bujard 1992). The activator plasmid expressed an artificial transcription factor activator (Z1 eTF, SEQ ID NO: 84) targeting the Z1 18-bp DNA element or a transcription factor activator which would not target the Z1 18-bp DNA element (“ΔZ1 eTF”, SEQ ID NO: 95). Twenty-four hours following transfection, cells were lysed and diluted 1:1000 in ONE-Glo EX Luciferase Assay buffer (Promega). Diluted lysate was assayed for Firefly and NanoLuc luminescence according to manufacturer's protocols (Nano-Glo Dual Luciferase Reporter Assay system, Promega), and relative light units (RLU) were measured on a bioluminescent plate reader (GloMax, Promega). Each transfection was performed in triplicate, and data reflect the average of triplicate wells (Mean±Standard Deviation).

FIG. 4 shows that no significant signal is seen with either reporter alone, reporter and a non-targeting transcription factor (Reporter+ΔZ1 eTF), or a TRE response element and a Z1 targeting transcription activator (ΔReporter+Z1 eTF). A strong response was only seen in the condition comprising a reporter containing a Z1 element and a Z1 targeting transcription activator (Reporter+Z1 eTF), indicating a specific interaction between the Z1 eTF and the Z1 element.

### Example 4

This example describes the generation of reporter constructs comprising a Z1-based response element with a single reporter (enhanced Green Florescent Protein (EGFP) coding sequence) and dual reporter system.

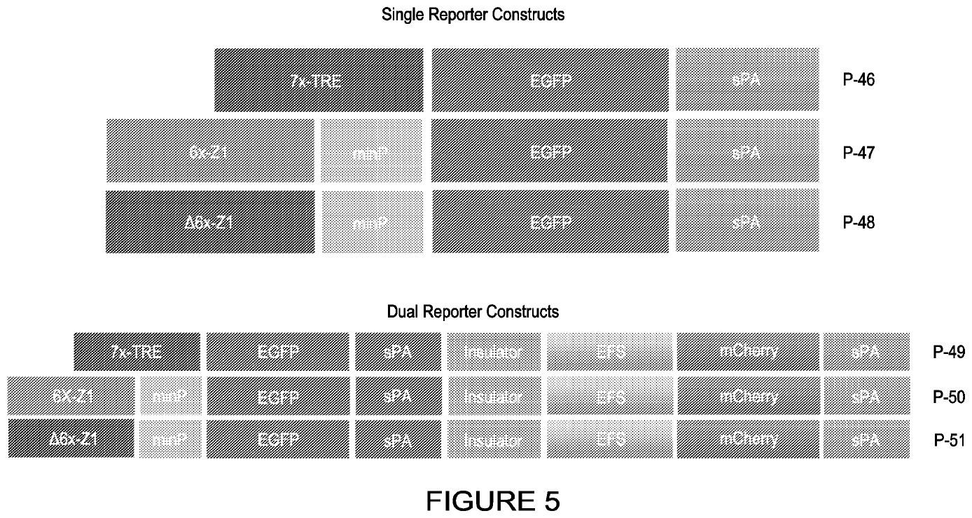

Single reporter constructs comprising a single enhanced GFP coding cassette (sPA=polyA signal sequence) and dual reporter constructs comprising an EGFP coding cassette and an mCherry coding cassette were made using standard techniques. A schematic of the different single and dual constructs is shown in FIG. 5.

Single reporter constructs were assayed as follows: HEK293T cells were cultured by standard methods, and each well of a 96-well plate was triply transfected (FugeneHD) with a reporter plasmid (50 ng), and an activator plasmid (50 ng) or non-fluorescent plasmid filler (50 ng) (100 ng total plasmid). The Z1-based reporter plasmid contained a Z1-Response Element (6×z1-mPro) upstream of the EGFP open reading frame (P-47). This 6×Z1-mPro element comprised six 18 bp Z1 target DNA sites with 5 bp spacer sequences, concatenated upstream of a minimal promoter element (mPro). A control reporter plasmid (P-48) was identical to P-47 except that the 18 bp Z1 target element was replaced with an alternative 18 bp sequence (Δ6×Z1-mPro). Twenty four hours following transfection, plates were imaged for EGFP and mCherry fluorescence (ImageXpress DLR, Molecular Devices). Fluorescence was detected only in cells transfected with the Z1-based reporter plasmid (6×Z1-mPro) operably linked to the EGFP open reading frame (P-47). No fluorescence was observed using the control plasmid lacking the intact Z1 response element.

Dual reporter constructs were assayed as described above for single reporter constructs, except the Z1 reporter and control plasmids also contain a second expression cassette containing an mCherry element under the control of the constitutive EF1a short (EFS) promoter. To allow for independent expression, this constitutive expression cassette was separated from the reporter expression cassette by an insulator sequence containing a human beta-globin transcriptional termination sequence (hACTB) and a Chicken beta-globin insulator (cHS4). mCherry-based fluorescence was detected in tested samples, demonstrating that a second reporter nucleic acid under control of a separate promoter is functional in the context of the system of the disclosure. Like the single reporter assay, EGFP-based fluorescence was detected only in cells transfected with the Z1-based reporter plasmid (6×Z1-mPro) operably linked to the EGFP open reading frame (P-47). No EGFP fluorescence was observed using the control plasmid lacking the intact Z1 response element.

### Example 5

This example describes the production and characterization of cells that stably express wild-type AAV receptor (AAVR). In vitro transduction efficiency of AAV9 is low in immortalized cell lines. To address this, cell lines were engineered to significantly enhance AAV9 transduction using AAVR.

HEK-293T (ATCC), HeLa-RC32 cells (ATCC), CHO-Lec2 (ATCC) and all derivatives were grown in media supplemented with 10% fetal calf serum (FCS) (Sigma, St. Louis), 3% L-Alanyl-L-Glutamine (Corning), and 1% NEAA (Corning), and grown in a humidified incubator at 37° C. with 5% CO₂. Purified, titred stocks of adeno-associated virus (AAV) serotypes 1, 3, 5, 6, 8, 9, and DJ were either made in-house or purchased from Vector Biolabs. The AAV stocks were all ssDNA AAV vectors encoding a reporter (GFP).

Cell lines stably expressing AAVR were generated using lentiviral AAVR vectors. Recombinant lentivirus vectors comprising AAVR coding sequences were produced using a construct comprising the nucleic acid sequence of SEQ ID NO: 31 and lentivirus packaging plasmids (pMD2.G and psPAX2) as per the protocol from Chimera Bioengineering in HEK293T cells. Vector was harvested in cell supernatant 48 hours post transfection and placed on the respective cell line to create a heterogeneous stable population of cells overexpressing AAVR. Puromycin selection was used to isolate lentivirus-positive cells. Once Western blots confirmed overexpression of AAVR in the heterogeneous population, cells were single-cell sorted into 96-well plates using a BD FACS Ariall. The resulting single subclones were grown up over 14 days and screened for AAV transduction efficiency using AAV9-CBA-GFP. The top clones for each cell line were grown and frozen.

Cells stably expressing AAVR were seeded at 10,000 cells/well (96-well plate) or 100,000 cells (24-well plate) overnight. The cells were then infected with the AAV stocks at a multiplicity of infection (MOI) of specified viral genomes/cell in complete DMEM. Virus infectivity was determined 48 hours post infection by measuring transgene expression, either through flow cytometry (fluorescence) or mRNA levels (RT-qPCR).

Flow cytometry: To measure GFP expression post AAV-GFP infection, cells were trypsinized 48 hours after infection and run through a BD FACS Melody to detect fluorescent cells. Uninfected cells were used as a negative control. Two parameters were evaluated, namely the percentage of cells that were GFP-positive (% infection) and mean fluorescent intensity (average fluorescence per cell).

RNA extractions and qPCR: RNA was extracted from respective cell pellets using the RNAeasy mini kit (Qiagen) following the manufacturer's instructions, and was then used to synthesize cDNA with SuperScript IV Reverse Transcriptase (Invitrogen). Quantitative PCR was performed using Lamin A/C, Lamin A, or Lamin C-specific primer sets, to evaluate expression levels of the respective genes.

Western blots: Cell pellets of 2×10⁶ cells were lysed with Laemmli SDS sample buffer containing 5% B-mercaptoethanol and boiled for 10 minutes at 95° C. Lysates were separated by SDS-PAGE using the Mini-Protean system (Bio-Rad) on 4-15% polyacrylamide gradient gels (Bio-Rad). Proteins were transferred onto nitrocellulose membranes (Bio-Rad) using the Bio-Rad Transblot protein transfer system in a semi-wet preparation. Membranes were blocked by incubating with 1×PBS buffer containing 5% non-fat milk for 1 hr at room temperature (RT). Membranes were subsequently incubated overnight at 4° C. with primary antibodies at a dilution of 1:1000 (anti-KIAA0319L antibody) or 1:2000 (anti-GAPDH antibody) in blocking buffer. Membranes were washed three times for 5 min using wash buffer (1×PBS buffer with 0.1% Tween-20), and further incubated in HRP-conjugated secondary antibodies (anti-mouse and anti-rabbit-1:5000 in blocking buffer) (GeneTex) for 1 hr at RT. After another set of three washes, antibody-bound AAVR (expected band at 150 kD) was visualized on a chemiluminescent reader.

RNA extractions, cDNA production and Two-color qPCR: RNA was extracted from respective infected cells (24-well format) using the RNAeasy mini kit (Qiagen) following the manufacturer's instructions and was then used to synthesize cDNA with SuperScript™ VILO™ CDNA Synthesis Kit with ezDNase™ Enzyme (Invitrogen). Two-color quantitative PCR was performed using TaqMan Fast Advanced Mastermix and VP64 or GAPDH (housekeeping) probes. 2^{-ddCt} was calculated to determine relative fold change.

### Results

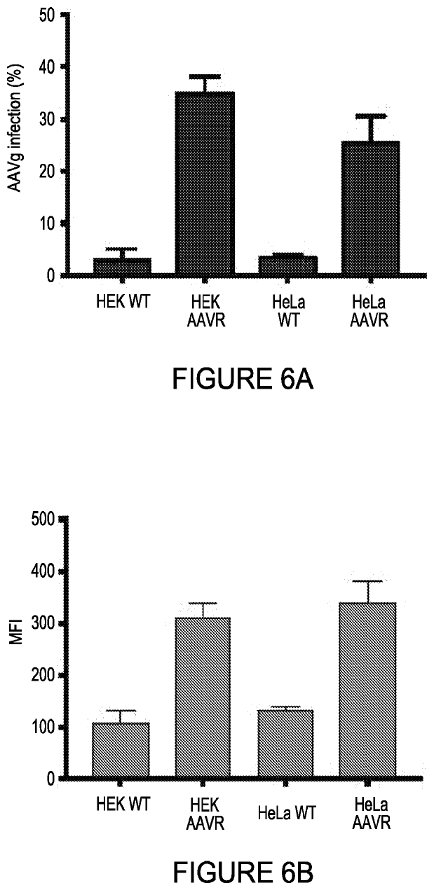

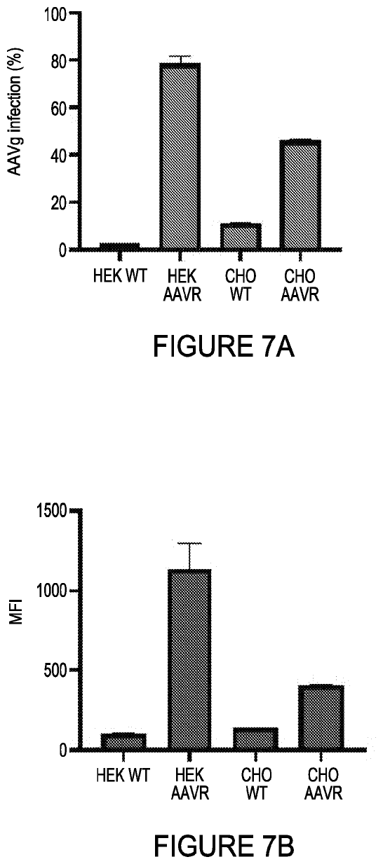

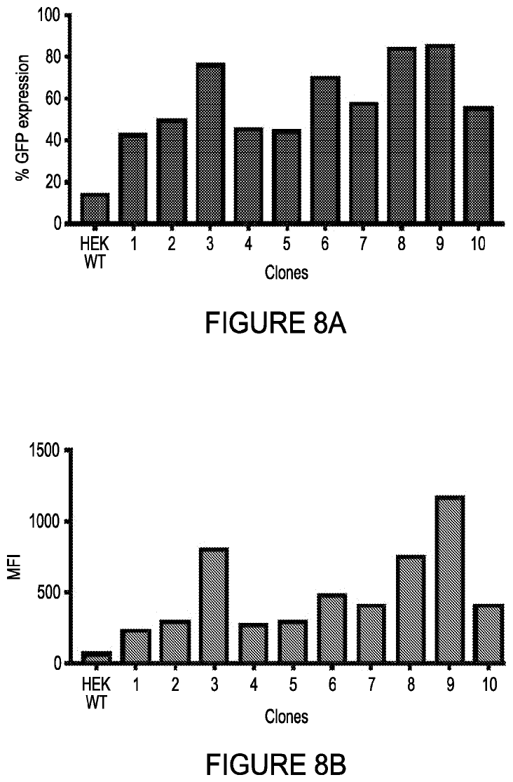

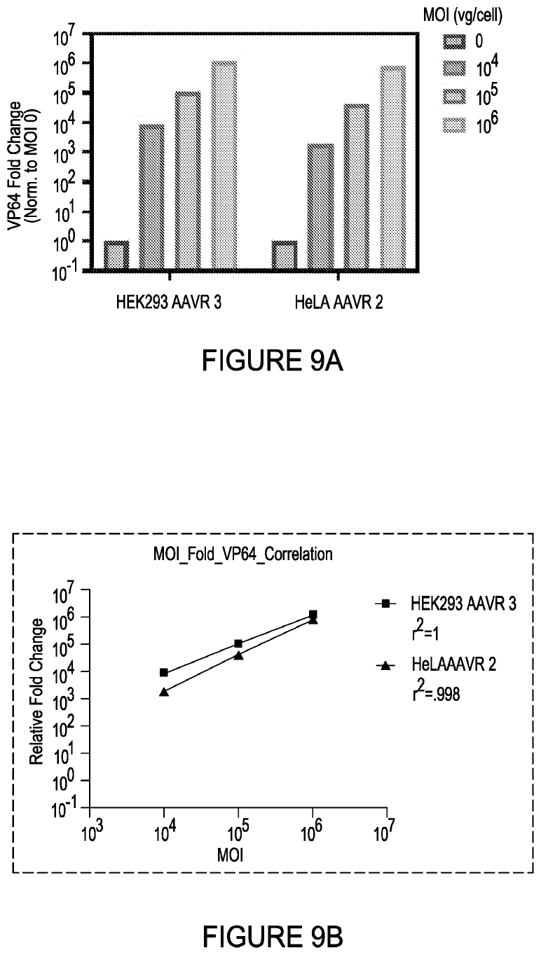

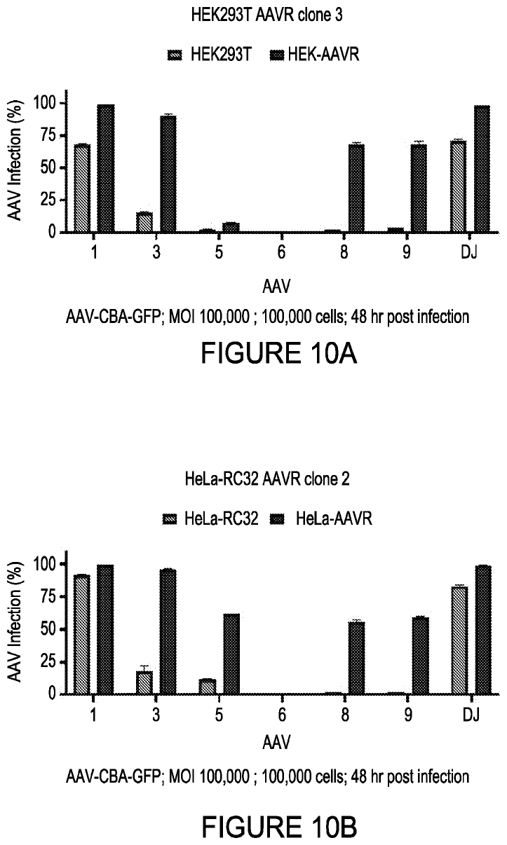

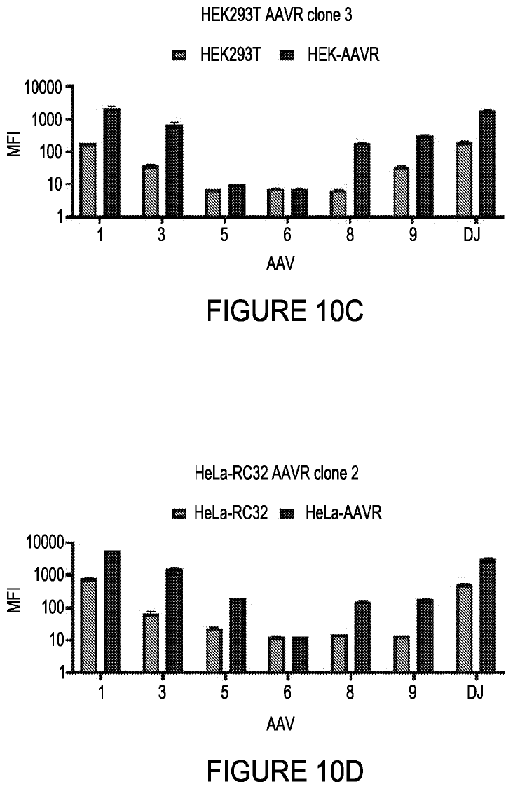

FIGS. 6A and 6B illustrate the results of overexpression of AAVR in HEK293T cells and HeLa-RC32 cells. AAV9-EF1a-GFP-KASH was provided at a multiplicity of infection (MOI) of 50,000. Cells were harvested at 48 hr post-infection, and the tested sample comprised 30,000 cells. Overexpression of AAVR yielded an 8-fold to 10-fold increase in GFP-positive cells (i.e., AAV+ transduced cells) (FIG. 6A), and also resulted in about a three-fold increase in expression of GFP (FIG. 6B; MFI=mean fluorescence intensity). FIGS. 7A and 7B illustrate the results of overexpression of AAVR in HEK293T cells and CHO-Lec2 cells. AAV9-EF1a-GFP-KASH was provided at a multiplicity of infection (MOI) of 10,000. Cells were harvested at 48 hr post-infection, and the tested sample comprised 30,000 cells. Overexpression of AAVR yielded a four-fold increase in GFP-positive CHO-Lec2 cells, while engineered HEK-293T cells demonstrated 35-fold higher effect at this higher MOI and using a CAG promoter to drive GFP expression. The cells expressed significantly more GFP on average. Subclones of engineered cells which stably expressed AAVR demonstrated enhanced AAV9 transduction. See, e.g., FIGS. 8A and 8B relating to HEK293T cell subclones. Similar results were observed for HeLa-RC32 and CHO-Lec2 subclones. Enhanced infectivity was demonstrated for multiple AAV serotypes. See FIGS. 10A-10D. HEK293T cells and HeLa-RC32 cells stably overexpressing wild-type AAVR were exposed to different serotype AAV vectors encoding GFP, and AAV infection rates were examined. Infectivity was enhanced for AAV-1, AAV-3, AAV-5, AAV-8, AAV-9, and AAV-DJ in both cell types tested when stably expressing AAVR, with AAV-3, AAV-8, and AAV-9 demonstrating about a 10-fold improvement in transduction. Mean fluorescent intensity data complemented the transduction data (FIGS. 10C-10D), showing a correlation between AAV infection rates and transgene expression.

### Example 6

This example demonstrates the production of cells which stably overexpress AAVR. HeRC32-AAVR, was engineered to significantly enhance AAV9 transduction. This cell line stably expresses AAVR, a cellular receptor that was identified as an essential host factor for AAV transduction.

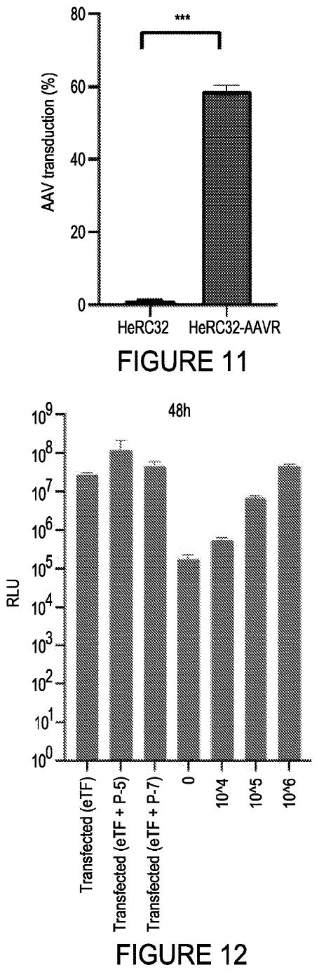

In particular, the HeRC32-AAVR cell line was generated by stable transduction of a lentivirus overexpressing the human AAVR gene under an EFS promoter (P-64) into the HeRC32 cell line (ATCC, Cat #CRL-2972). Positively transduced cells were selected using puromycin, and the resulting heterogenous population was single cell sorted to isolate clonal populations overexpressing AAVR. These clones were screened based on AAV9 transduction efficiency. The respective cell lines were infected with AAV9-CBA-GFP at an MOI 100,000 for 48 hours, and analyzed by flow cytometry for GFP expression. Percentage of GFP-positive cells was used to measure AAV transduction. As seen in FIG. 11, the selected clone demonstrated greater than a fifty-fold transduction compared to the parent HeRC32 cell line.

Candidate reporter cell lines were then generated by stable transduction of HeRC32-AAVR cells with lentivirus for each of three reporter transgenes: 1) P-6, which includes the 7× concatenated Z1 response element in the V1 dual reporter format, 2) P-8, which includes the 7× concatenated Z1 response element in the V2 standalone reporter format, or 3) P-13, which includes Z1 substituted TRE response element in the V2 standalone reporter format. The V1 dual reporter format comprises a dual-expression vector, where Nluc is expressed under the control of the Z1 response element, and firefly luciferase is expressed under the control of an independent ubiquitous promoter element allowing for an internal control. The resulting heterogenous stable cell lines (referred to as HeRC32-AAVR-P-6, HeRC32-AAVR-P-8, HeRC32-AAVR-P-13) were then further evaluated for reporter expression and assay feasibility.

An initial assay was performed in the HeRC32-AAVR-P-8 heterogenous cell line. These cells were plated, at an initial density of 10,000 cells/well, in a 96-well plate and an AAV encoding an eTF activator (SEQ ID NO: 84) was added over a 3-point MOI series from 1E4 to 1E6 genome copies/cell. As positive control conditions, the reporter cell line alone was transfected with activator (eTF, SEQ ID NO: 84) plasmid alone, or co-transfected reporter plasmid, either in the original screening vector (P-5), or the lentiviral packaging plasmid (P-8).

Cells were assayed 48 h following eTF activator (SEQ ID NO: 84) transduction. Luciferase assays were performed as described previously and cell lysates were undiluted to account for lower overall signal due to reduced reporter copy number and AAV transduction.

As seen in FIG. 12, strong reporter induction was observed above baseline in the reporter plasmid transfection conditions (P-5 or P-8) and activator (eTF) plasmid transfection conditions, and the response to infection with an AAV encoding eTF activator (SEQ ID NO: 94) increased with increasing MOI, as expected.

### Example 7

This example demonstrates the production of cells which stably overexpress AAVR and comprise a response element-reporter nucleic acid stably incorporated into the cellular genome. In particular, luciferase reporter-expressing, stable cell lines (AAVR-Reporter HeLa) were created using a lentivirus vector to integrate a nucleic acid of the disclosure into the genome of HeLa-AAVR cells which constitutively express the AAVR gene.

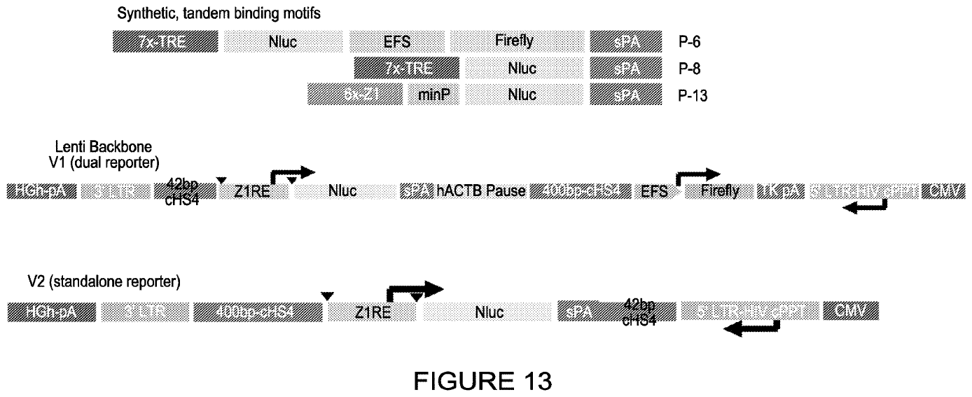

Different lentiviral constructs were made as illustrated in FIG. 13. One backbone (V2) contained a response element-reporter cassette, while another backbone (V1) contained the response element-reporter cassette with an additional reporter cassette. The constructs contained a Z1-based response element (6×z1-mPro or 7×z1-TRE (P-8)) upstream of the open reading frame for luciferase. A third Z1-based response element (P-6) was constructed which contained the same response element as P-8 (7×z1-TRE-Nluc), but further contained a second expression cassette comprising a firefly luciferase element under the control of the constitutive EF1a short (EFS) promoter. The response element-reporter cassette is flanked by cHS4 insulator elements, and this entire cassette is expressed in a lentivirus expression cassette in an inverted orientation, such that it is antisense to the lentivirus cassette. With respect to response element-reporter cassette P-6, the constitutive expression cassette encoding firefly luciferase was separated from the response element-Nluc cassette by an insulator sequence containing a human beta-globin transcriptional termination sequence (hACTB), and a Chicken beta-globin insulator (cHS4).

Lentivirus-transduced AAVR-Reporter Hela cells were cultured as a heterogeneous population by standard methods. Cells were transduced with AAV expressing a Z1 targeting transcription factor (SEQ ID NO: 94) at varying multiplicity of infection (MOI) of 1×10⁴, 3×10⁴, 1×10⁵, 3×10⁵, 1×10⁶, 3×10⁶ as a reference standard (RS). To test known potency samples, the same vector was repeated as an assay control (AC), or diluted to 70% and 40% before undergoing the same dilution series. After 48 h, cells were lysed and assayed for Firefly and NanoLuc luminescence according to manufacturer's protocols (Nano-Glo Dual Luciferase Reporter Assay system, Promega). Relative luminescence (RLU) was measured on a bioluminescent plate reader (GloMax, Promega). Each transfection was performed in triplicate; data reflect the average of triplicate wells (Mean±Standard Deviation).

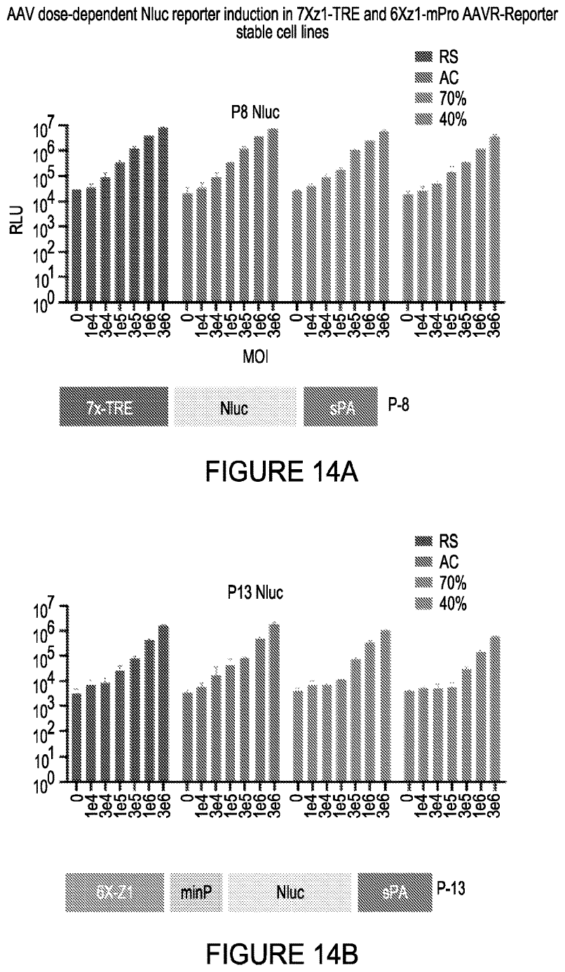

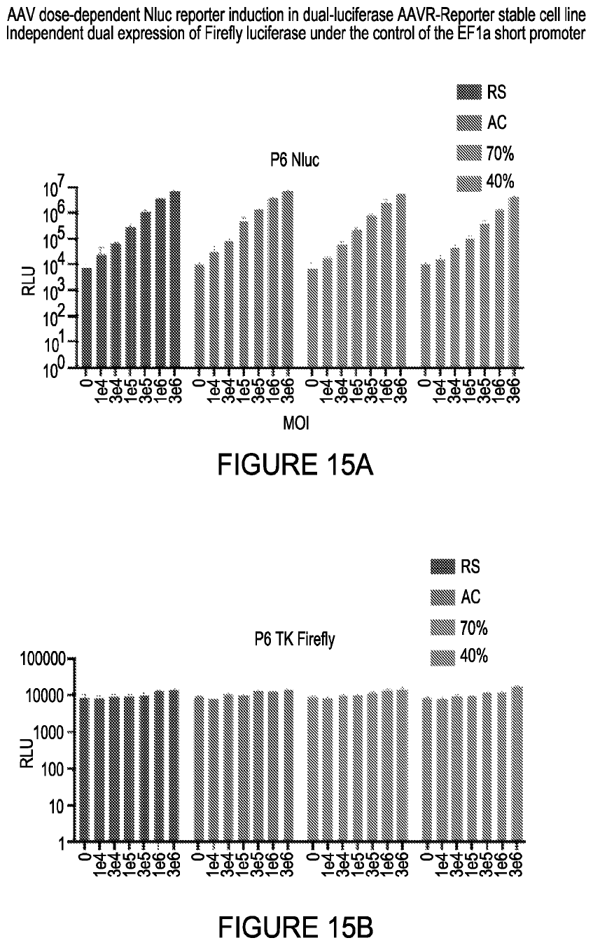

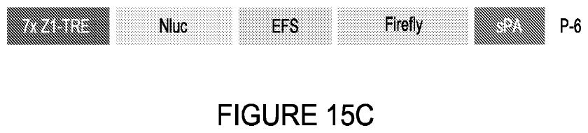

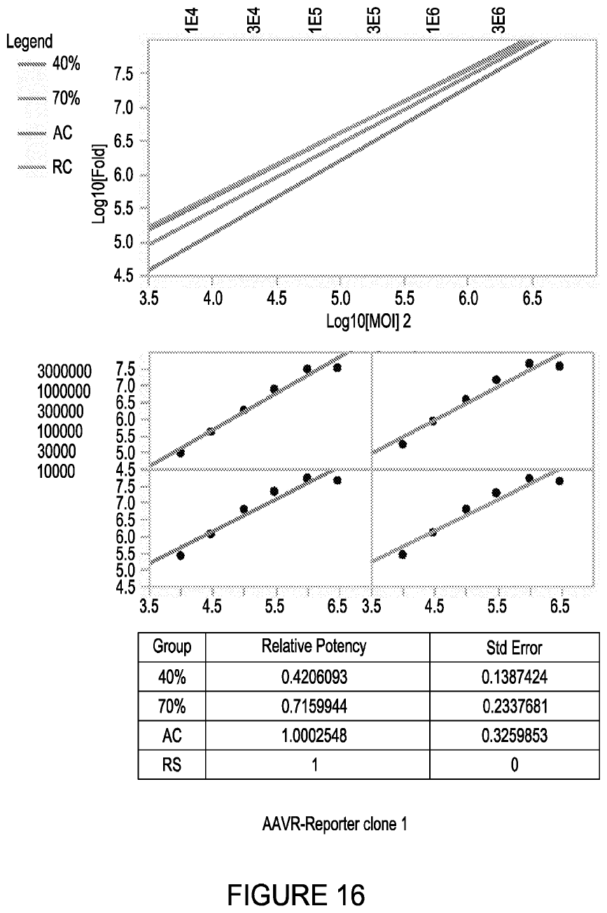

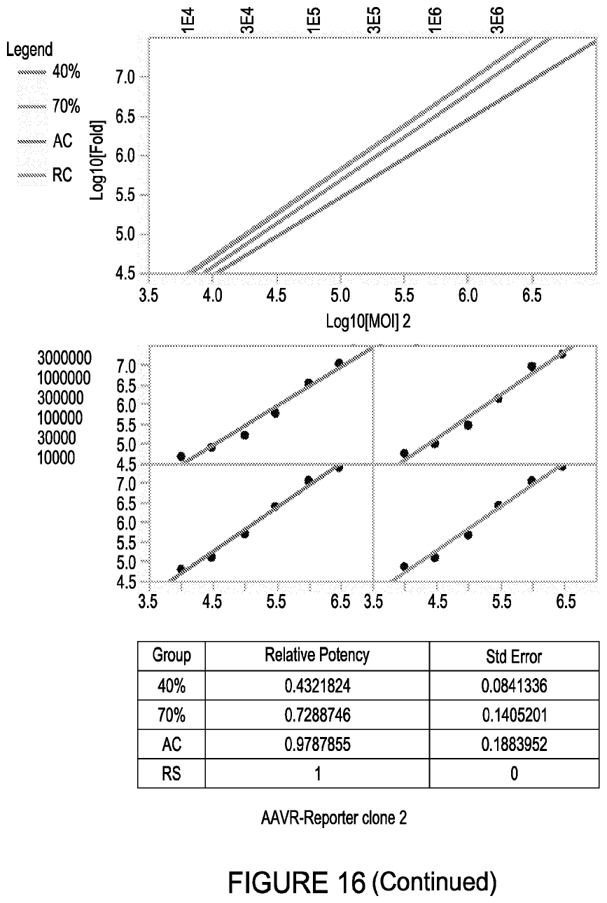

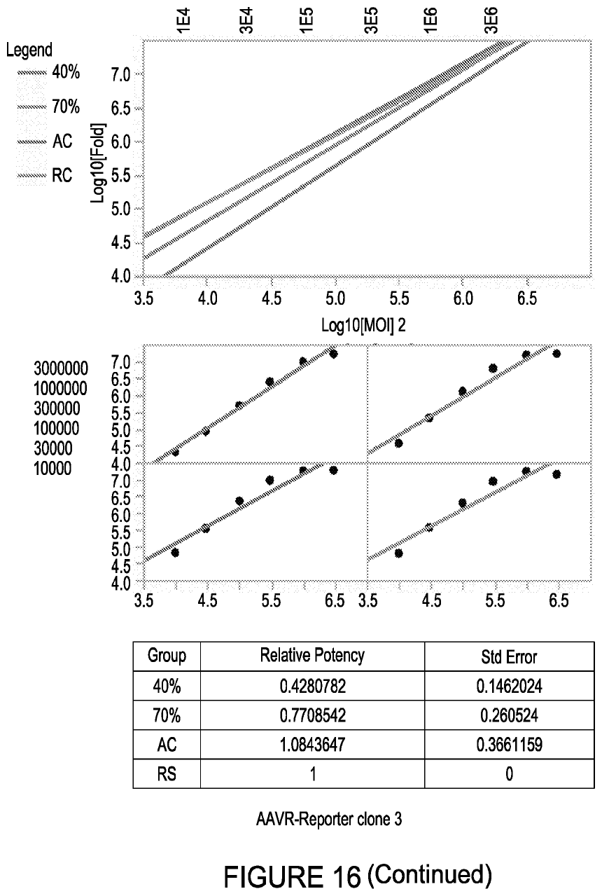

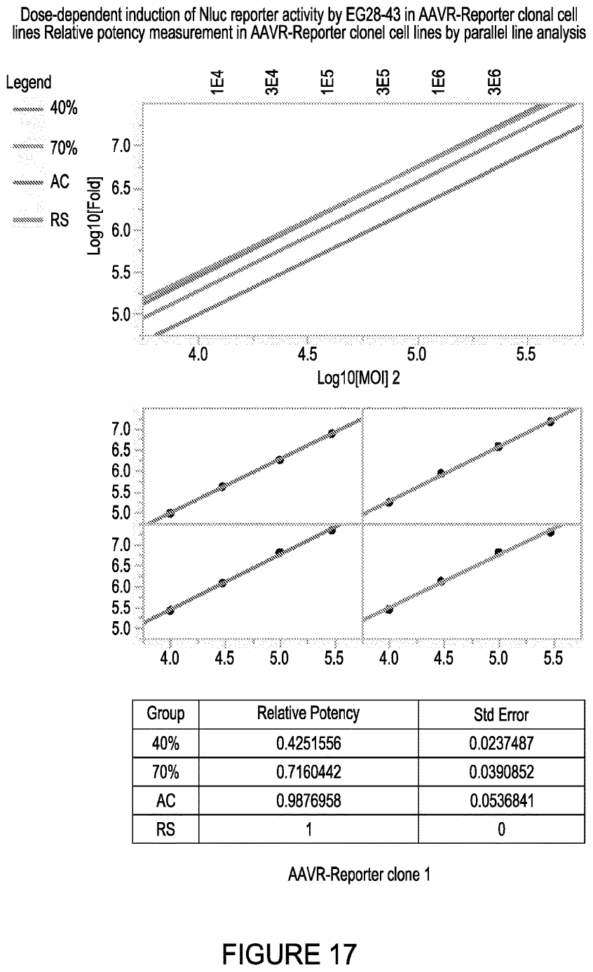

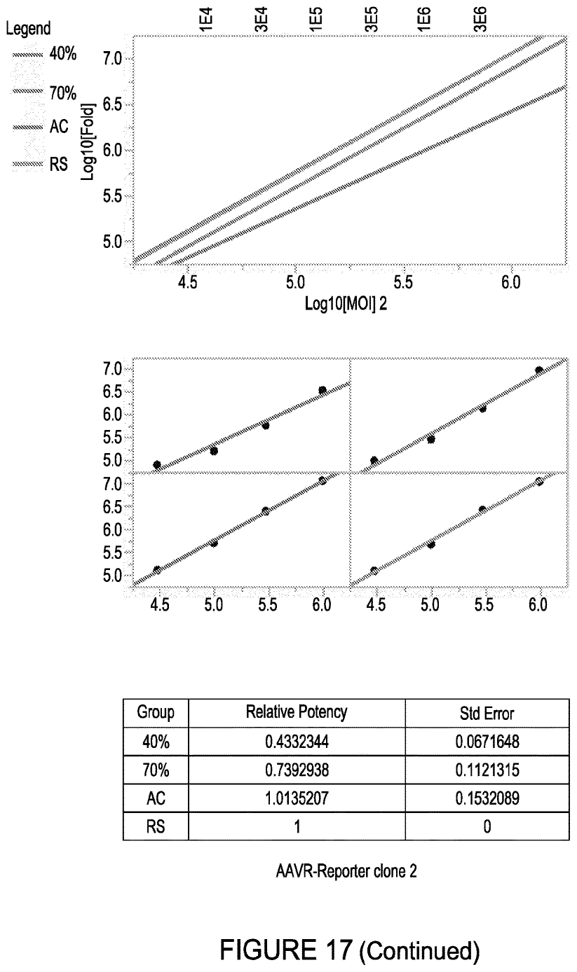

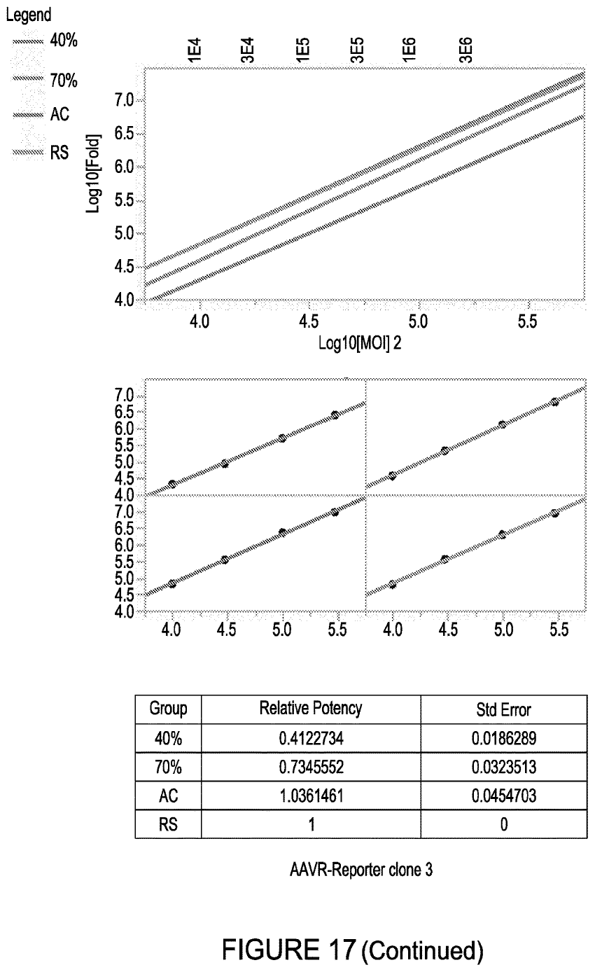

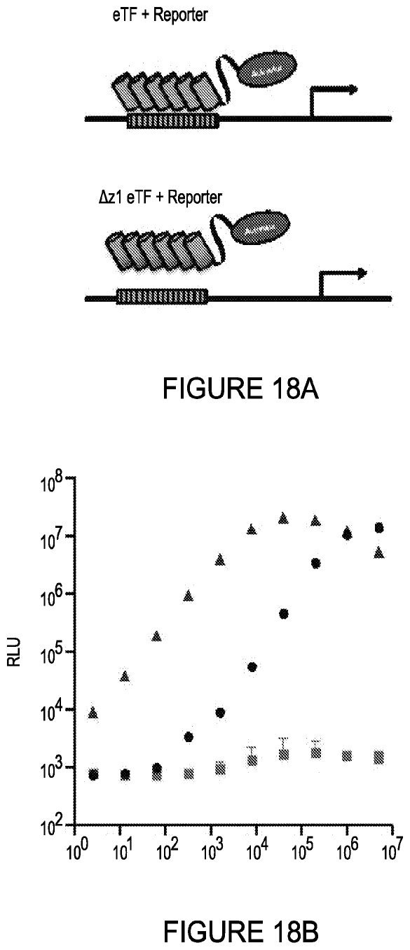

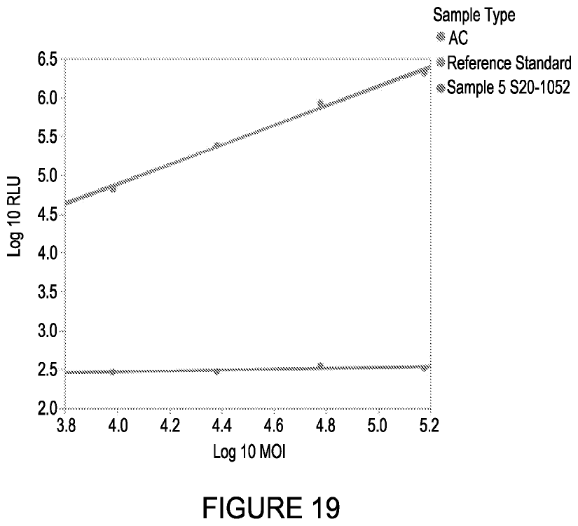

The results for illustrated in FIGS. 14A-14B and FIGS. 15A-15B. As expected, eTF activator (SEQ ID NO: 94) dose-dependent Nluc expression was observed across all three cell lines. The dual reporter line, HeRC32-AAVR-P-6, produced constitutive expression of firefly luciferase, which showed only a minimal response to eTF activator (SEQ ID NO: 94) dose. To evaluate whether these dose responses can be used to assess relative potency, dose responses under different dilution conditions, linear regressions were applied to each dose response curve, and preliminary parallel line analysis was performed over the three test samples against the reference standard using log 10 MOI and log 10 RLU values (FIGS. 16 and 17). Generally, variability was observed across cell lines and experiments in the linearity of the dose responses; however, the response in each cell line can be described by a linear relationship, between Log 10 MOI and Log 10 RLU. Table 2 summarizes the parallel line analysis relative potency results, providing proof of concept for feasibility of relative potency measurements in the reporter cell lines.

TABLE 2

| | | | | 100% (AC) |
|---|---|---|---|---|
| Cell Line | Experiment | 40% RP | 70% RP | RP |
| AAVR-Reporter | 1 | 0.43 | 1.03 | 1.05 |
| clone 1 | 2 | 0.47 | 0.75 | 1.22 |
| AAVR-Reporter | 1 | 0.61 | 0.87 | 0.91 |
| clone 2 | 2 | 0.42 | 0.78 | 0.96 |
| AAVR-Reporter | 1 | 0.38 | 1.18 | 1.14 |
| clone 3 | 2 | 0.39 | 0.72 | 1.26 |

Table 2: Summary of relative potency in samples tested in candidate cell lines by parallel line analysis (PLA): Assay control (AC), 40% relative potency samples (40%), 70% relative potency samples (70%), across two independent experiments.

To confirm reporter specificity to AAV in the final HeRC32-AAVR-Reporter cell line, the reporter response was compared to the AAV9 carrying the z1-targeted eTF under the control of the strong ubiquitous CBA promoter, to a control AAV, identical except for a sequence mutation in the z1-targeting DNA-binding domain of the eTF (AAV9-CBA-Δz1-eTF). The AAV9-CBA-z1-eTF construct should produce a robust dose response, since it expresses the Z1-targeted eTF transgene. In contrast, the DNA binding mutant vector (AAV9-CBA-Δz1-eTF) should not produce reporter activity, as the reporter response element cassette contains the specific z1 eTF target sequence. In addition, the reference standard control sample was included, which contains the z1-targeted eTF expressed under the control of a GABA-selective promoter element (SEQ ID NO: 94). Because of the strength of the CBA promoter compared to the GABA selective promoter, the AAV9-CBA-z1eTF construct was expected to produce a stronger induction. Therefore, a full 10-point dose curve was included in order to capture the linear range of all samples. A robust dose-dependent reporter activation was observed in response to AAV9-CBA-z1-eTF, see FIG. 16B (triangles). The dose response exhibited a leftward shift compared to the reference standard (circles), due to the much stronger CBA promoter. In contrast, the control AAV9-CBA-Δz1-eTF sample (squares) did not activate reporter activity. Together, these data demonstrate assay specificity to the eTF activator, and the requirement for sequence-dependent eTF-DNA target interaction for reporter activation.

Additionally, to further confirm assay specificity, using the same promoter and vector design as SEQ ID NO: 94, a control AAV9 vector was generated, identical to SEQ ID NO: 94 except for a mutated sequence in the six 7-amino acid zinc finger DNA-binding domain that defines the 18 bp target DNA sequence. In contrast to the reference standard or assay control (AC) vectors, this control sample did not activate reporter activity, see FIG. 17. Therefore, this assay is specific to the eTF of SEQ ID NO: 84.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All method steps described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Indeed, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document.

SEQUENCES

| SEQ | |
|---|---|
| NO: | SEQUENCE |
| 1 | ctaggtcaagtgtaggag |
| 2 | CTCGAT |
| 3 | GATAGGGAGTAAACTCGA |
| 4 | [GGGS]n |
| 5 | [GGGGS]n |
| 6 | [GGSG]n |
| 7 | GGSGGGSG |
| 8 | TGCRCNC |
| | R is A or G; N is any base |
| 9 | GAGTTTACTCCCTATCAGTGATAGAGAACGTATGTCGAGTTTACTCCCTATCAGTGATAG |
| | AGAACGATGTCGAGTTTACTCCCTATCAGTGATAGAGAACGTATGTCGAGTTTACTCCC |
| | TATCAGTGATAGAGAACGTATGTCGAGTTTACTCCCTATCAGTGATAGAGAACGTATGTC |
| | GAGTTTATCCCTATCAGTGATAGAGAACGTATGTCGAGTTTACTCCCTATCAGTGATAGA |
| | GAACGTATGTCGAGGTAGGCGTGTACGGTGGGAGGCCTATATAAGCAGAGCTCGTTTA |
| | GTGAACCGTCAGATCGCC |
| 10 | GAGTTTACTCCCTATCctaggtcaagtgtaggagTCGAGTTTACTCCCTATCctaggtcaagtgtaggag |
| | TCGAGTTTACTCCCTATCctaggtcaagtgtaggagTCGAGTTTACTCCCTATCctaggtcaagtgtagg |
| | agTCGAGTTTACTCCCTATCctaggtcaagtgtaggagTCGAGTTTATCCCTATCctaggtcaagtgtag |
| | gagTCGAGTTTACTCCCTATCctaggtcaagtgtaggagTCGAGGTAGGCGTGTACGGTGGGAG |
| | GCCTATATAAGCAGAGCTCGTTTAGTGAACCGTCAGATCGCC |
| 11 | TCCCTATCctaggtcaagtgtaggagTCGAGTTTACTCCCTATCctaggtcaagtgtaggagTCGAGTTT |
| | ACTCCCTATCctaggtcaagtgtaggagTCGAGTTTATCCCTATCctaggtcaagtgtaggagTCGAGTT |
| | TACTCCCTATCctaggtcaagtgtaggagTCGAGGTAGGCGTGTACGGTGGGAGGCCTATATAA |
| | GCAGAGCTCGTTTAGTGAACCGTCAGATCGCC |
| 12 | TCCCTATCctaggtcaagtgtaggagTCGAGTTTATCCCTATCctaggtcaagtgtaggagTCGAGTTTA |
| | CTCCCTATCctaggtcaagtgtaggagTCGAGGTAGGCGTGTACGGTGGGAGGCCTATATAAGC |
| | AGAGCTCGTTTAGTGAACCGTCAGATCGCC |
| 13 | TCCCTATCctaggtcaagtgtaggagTCGAGGTAGGCGTGTACGGTGGGAGGCCTATATAAGCA |
| | GAGCTCGTTTAGTGAACCGTCAGATCGCC |
| 14 | ctaggtcaagtgtaggagatttgcctaggtcaagtgtaggagCTCGATctaggtcaagtgtaggagCTCGATctaggtca |
| | agtgtaggagatttgcctaggtcaagtgtaggagCTCGATctaggtcaagtgtaggagGCTAGCTAGAGGGTATA |
| | TAATGGAAGCTCGACTTCCAG |
| 15 | ctaggtcaagtgtaggagCTCGATctaggtcaagtgtaggagCTCGATctaggtcaagtgtaggagGCTAGCTAG |
| | AGGGTATATAATGGAAGCTCGACTTCCAG |
| 16 | AGTGATAGAGAACGTATGatttgcAGTGATAGAGAACGTATGCTCGATAGTGATAGAGAAC |
| | GTATGCTCGATAGTGATAGAGAACGTATGatttgcAGTGATAGAGAACGTATGCTCGATAG |
| | TGATAGAGAACGTATGGCTAGCTAGAGGGTATATAATGGAAGCTCGACTTCCAG |
| 17 | ctaggtcaagtgtaggagatttgcctaggtcaagtgtaggagCTCGATctaggtcaagtgtaggagCTCGATctaggtca |
| | agtgtaggagatttgcctaggtcaagtgtaggagCTCGATctaggtcaagtgtaggagGCTAGCaaaaggctgagaga |
| | aaagaggttgaggaagaaatcataaatctggattgtgagaaagtgtttaatatttagccactagatggcgatgtaatgtaaggtgct |
| | gtcttgactttttttttttttttttgaaacaagctatttgctgatttgtattaggtaccatagagtgaggcgaggatgaagccgagaggatac |
| | tgcagaggtctctggtgcatgtgtgtatgtgtgcgtttgtgtgtgtttgtgtgtctgtgtgttctgccccagtgagactgcagcccttgtaaa |
| | tactttgacaccttttgcaagaaggaatctgaacaattgcaactgaaggcacattgttatcatctcgtctttgggtgatgctgttcctcac |
| | tgcagatggataattttccttt |
| 18 | ctaggtcaagtgtaggagatttgcctaggtcaagtgtaggagCTCGATctaggtcaagtgtaggagCTCGATctaggtca |
| | agtgtaggagatttgcctaggtcaagtgtaggagCTCGATctaggtcaagtgtaggagGCTAGCgcctttatacgcacag |
| | tctccatcttctgggtgtgtcacagactgaataactcattagtgatatagtgttaattagcctaatgtactttgattaaggccatatttgaa |
| | ccacttttaaaactcatggcacagttcctgtatcagtaaagcacaagaattaataaataagtgatgcttaactaaactcaacacag |
| | aaaccatttgtgttaaaattttttctttagaaatcacctttcaatttaaggagaaaacagactttaaatcctctagctcatgtttcatgaca |
| | agaatttatttatattaacatctcttagtccactctttaaaatatctgtattccttttattttaggaatttcatatgcagaataaatggtaa |
| | ttaaaatgtgcaggatgacaag |
| 19 | ctaggtcaagtgtaggagCTCGATctaggtcaagtgtaggagCTCGATctaggtcaagtgtaggagGCTAGCaaaa |
| | ggctgagagaaaagaggttgaggaagaaatcataaatctggattgtgagaaagtgtttaatatttagccactagatggcgatgta |
| | atgtaaggtgctgtcttgactttttttttttttttttgaaacaagctatttgctgatttgtattaggtaccatagagtgaggcgaggatgaagc |
| | cgagaggatactgcagaggtctctggtgcatgtgtgtatgtgtgcgtttgtgtgtgtttgtgtgtctgtgtgttctgccccagtgagactgc |
| | agcccttgtaaatactttgacaccttttgcaagaaggaatctgaacaattgcaactgaaggcacattgttatcatctcgtctttgggtg |
| | atgctgttcctcactgcagatggataattttccttt |
| 20 | ttcatgatttccatgcaaactcccagcctgcctggctgcctgctgctgccaatacttcttgctcttgctggtgatgacattcagagagct |
| | ccccagcactggtgcttcgttatgtaaggctgtctaggtcaagtgtaggagacacactgctggcctgtggaaactcatggaactgtt |
| | cctccagattaacacttcaggggttatggaagctggaggaagctgagcttttactacatcttttgggg |
| 21 | caaaatatttcaaaagaggaagacaagaaagacttcatgatttccatgcaaactcccagcctgcctggctgcctgctgctgccaa |
| | tacttcttgctcttgctggtgatgacattcagagagctccccagcactggtgcttcgttatgtaaggctgtctaggtcaagtgtaggag |
| | acacactgctggcctgtggaaactcatggaactgttcctccagattaacacttcaggggttatggaagctggaggaagctgagctt |
| | ttactacatcttttggggGCTAGCaaaaggctgagagaaaagaggttgaggaagaaatcataaatctggattgtgagaaagtgtttaa |
| | tatttagccactagatggcgatgtaatgtaaggtgctgtcttgactttttttttttttttttgaaacaagctatttgctgatttgtattagg |
| | taccatagagtgaggcgaggatgaagccgagaggatactgcagaggtctctggtgcatgtgtgtatgtgtgcgtttgtgtgtgtttgt |
| | gtgtctgtgtgttctgccccagtgagactgcagcccttgtaaatactttgacaccttttgcaagaaggaatctgaacaattgcaactga |
| | aggcacattgttatcatctcgtctttgggtgatgctgttcctcactgcagatggataattttccttt |
| 22 | caaaatatttcaaaagaggaagacaagaaagacttcatgatttccatgcaaactcccagcctgcctggctgcctgctgctgccaa |
| | tacttcttgctcttgctggtgatgacattcagagagctccccagcactggtgcttcgttatgtaaggctgtctaggtcaagtgtaggag |
| | acacactgctggcctgtggaaactcatggaactgttcctccagattaacacttcaggggttatggaagctggaggaagctgagctt |
| | ttactacatcttttggggGCTAGCGGTACCgcctttatacgcacagtctccatcttctgggtgtgtcacagactgaataactca |
| | ttagtgatatagtgttaattagcctaatgtactttgattaaggccatatttgaaccacttttaaaactcatggcacagttcctgtatcagta |
| | aagcacaagaattaataaataagtgatgcttaactaaactcaacacagaaaccatttgtgttaaaattttttctttagaaatcacctttc |
| | aatttaaggagaaaacagactttaaatcctctagctcatgtttcatgacaagaatttatttatattaacatctcttagtccactctttaaaa |
| | tatctgtattccttttattttaggaatttcatatgcagaataaatggtaattaaaatgtgcaggatgacaag |
| 23 | caaaatatttcaaaagaggaagacaagaaagacttcatgatttccatgcaaactcccagcctgcctggctgcctgctgctgccaa |
| | tacttcttgctcttgctggtgatgacattcagagagctccccagcactggtgcttcgttatgtaaggctgtctaggtcaagtgtaggag |
| | acacactgctggcctgtggaaactcatggaactgttcctccagattaacacttcaggggttatggaagctggaggaagctgagctt |
| | ttactacatcttttggggGCTAGCaaaaggctgagagaaaagaggttgaggaagaaatcataaatctggattgtgagaaagtgtttaat |
| | atttagccactagatggcgatgtaatgtaaggtgctgtcttgactttttttttttttttttgaaacaagctatttgctgatttgtattagg |
| | taccatagagtgaggcgaggatgaagccgagaggatactgcagaggtctctggtgcatgtgtgtatgtgtgcgtttgtgtgtgtttgt |
| | gtgtctgtgtgttctgccccagtgagactgcagcccttgtaaatactttgacaccttttgcaagaaggaatctgaacaattgcaactga |
| | aggcacattgttatcatctcgtctttgggtgatgctgttcctcactgcagatggataattttcctttGGTACCgcctttatacgcacagt |
| | ctccatcttctgggtgtgtcacagactgaataactcattagtgatatagtgttaattagcctaatgtactttgattaaggccatatttgaac |
| | cacttttaaaactcatggcacagttcctgtatcagtaaagcacaagaattaataaataagtgatgcttaactaaactcaacacaga |
| | aaccatttgtgttaaaattttttctttagaaatcacctttcaatttaaggagaaaacagactttaaatcctctagctcatgtttcatgacaa |
| | gaatttatttatattaacatctcttagtccactctttaaaatatctgtattccttttattttaggaatttcatatgcagaataaatggtaa |
| | ttaaaatgtgcaggatgacaag |
| 24 | ttcatgatttccatgcaaactcccagcctgcctggctgcctgctgctgccaatacttcttgctcttgctggtgatgacattcagagagct |
| | ccccagcactggtgcttcgttatgtaaggctgtAGTGATAGAGAACGTATGacacactgctggcctgtggaaactcatg |
| | gaactgttcctccagattaacacttcaggggttatggaagctggaggaagctgagcttttactacatcttttgggg |
| 25 | ttcatgatttccatgcaaactcccagcctgcctggctgcctgctgctgccaatacttcttgctcttgctggtgatgacattcagagagct |
| | ccccagcactggtgcttcgttatgtaaggctgtctaggtcaagtgtaggagacacactgctggcctgtggaaactcatggaactgtt |
| | cctccagattaacacttcaggggttatggaagctggaggaagctgagcttttactacatcttttggggGCTAGCTAGAGGG |
| | TATATAATGGAAGCTCGACTTCCAG |
| 26 | ttcatgatttccatgcaaactcccagcctgcctggctgcctgctgctgccaatacttcttgctcttgctggtgatgacattcagagagct |
| | ccccagcactggtgcttcgttatgtaaggctgtAGTGATAGAGAACGTATGacacactgctggcctgtggaaactcatg |
| | gaactgttcctccagattaacacttcaggggttatggaagctggaggaagctgagcttttactacatcttttggggGCTAGCTA |
| | GAGGGTATATAATGGAAGCTCGACTTCCAG |
| 27 | gggtggcatc cctgtgaccc ctccccagtg cctctcctgg ccctggaagt tgccactcca |
| | gtgcccacca gccttgtcct aataaaatta agttgcatca ttttgtctga ctaggtgtcc |
| | ttctataata ttatggggtg gaggggggtg gtatggagca aggggcaagt tgggaagaca |
| | acctgtaggg cctgcggggt ctattgggaa ccaagctgga gtgcagtggc acaatcttgg |
| | ctcactgcaa tctccgcctc ctgggttcaa gcgattctcc tgcctcagcc tcccgagttg |
| | ttgggattcc aggcatgcat gaccaggctc agctaatttt tgtttttttg gtagagacgg |
| | ggtttcacca tattggccag gctggtctcc aactcctaat ctcaggtgat ctacccacct |
| | tggcctccca aattgctggg attacaggcg tgaaccactg ctcccttccc tgtcctt |
| 28 | aataaaagat ctttattttc attagatctg tgtgttggtt ttttgtgtgc ggaccgcacg |
| | tg |
| 29 | LEPGEKP-[YKCPECGKSFS X HQRTH TGEKP]n-YKCPECGKSFS X HQRTH-TGKKTS |
| | n is an integer from 1-15, e.g., 1, 2, 3, 4, 5, 6, 7, 8, 9, 10, 11, 12, 13, 14, or 15, |
| | and each X independently is a recognition sequence (e.g., a recognition helix) capable |
| | of binding to 3 bp of a target sequence |
| 30 | atgcccgaac aggagcgcca gattactgcc agagagggag catccagaaa aatcctgagc |
| | aaactgtcac tgcccacacg agcttgggaa cccgcaatga agaaaagctt cgcctttgac |
| | aacgtgggat acgagggagg actggatgga ctgggaccta gctcccaggt ggccacctct |
| | acagtccgaa tcctgggcat gacttgccag agttgcgtga aatcaattga agaccggatc |
| | agtaatctga agggaatcat tagcatgaaa gtgtccctgg agcagggctc agccaccgtg |
| | aagtatgtcc ctagcgtggt ctgcctgcag caggtgtgcc accagatcgg cgatatgggg |
| | ttcgaggcct ccattgctga agggaaagcc gcttcttggc ctagccggtc cctgccagca |
| | caggaagcag tggtcaagct gagagtggag ggaatgacat gccagagctg cgtgagcagt |
| | atcgaaggaa aggtccgaaa actgcagggc gtggtccggg tgaaggtctc tctgagtaac |
| | caggaggccg tgattaccta ccagccctat ctgatccagc ctgaagacct gagggatcac |
| | gtgaatgaca tgggcttcga ggcagccatc aagtccaaag tggccccact gtctctgggg |
| | cccattgaca tcgaaagact gcagtccacc aacccaaaga ggcccctgtc aagcgccaac |
| | cagaacttca acaatagtga gaccctggga caccagggct cacatgtggt cacactgcag |
| | ctgaggattg acggcatgca ctgcaagtct tgcgtgctga acattgagga aaatatcggc |
| | cagctgctgg gggtgcagtc tatccaggtc agtctggaga acaagactgc tcaggtgaaa |
| | tacgatcctt catgcaccag cccagtggca ctgcagcgcg ctatcgaagc actgccccct |
| | ggaaatttca aggtgagcct gcctgacgga gcagagggat ccggaaccga tcacaggtcc |
| | tctagttcac attccccagg atctccacca cgaaaccagg tgcagggaac atgttccacc |
| | acactgattg caatcgccgg catgacttgc gcctcatgcg tgcacagcat tgaagggatg |
| | atctctcagc tggagggagt gcagcagatc tcagtcagcc tggccgaggg cactgctacc |
| | gtgctgtaca atcccagtgt catctcacct gaggaactgc gggctgcaat tgaggacatg |
| | gggttcgaag cttccgtggt ctccgaatct tgcagtacca accccctggg gaatcattcc |
| | gccggaaact ctatggtgca gactaccgac gggacaccta cttctgtgca ggaggtcgca |
| | ccacacacag gacgcctgcc agccaatcat gctcccgaca tcctggccaa aagcccccag |
| | tccacccggg ctgtggcacc tcagaagtgt tttctgcaga tcaaaggcat gacctgcgcc |
| | tcttgcgtga gcaacattga gcggaatctg cagaaggaag ctggggtgct gagcgtgctg |
| | gtcgcactga tggccggaaa ggctgagatc aagtacgacc ctgaagtgat ccagccactg |
| | gagattgccc agttcatcca ggatctgggc tttgaggccg ctgtgatgga agactatgct |
| | gggagcgatg gaaacattga actgaccatc acaggcatga cttgtgcctc ttgcgtgcac |
| | aacatcgaga gtaaactgac tagaaccaat gggattacct acgccagtgt ggccctggct |
| | acatcaaagg ctctggtgaa attcgacccc gagatcattg gacctaggga catcatcaag |
| | atcattgagg aaatcggctt tcacgcaagc ctggcccagc gcaacccaaa tgcccaccat |
| | ctggaccata agatggagat caagcagtgg aagaaaagtt tcctgtgctc actggtgttt |
| | ggaatccccg tcatggccct gatgatctac atgctgatcc ctagcaacga gccacaccag |
| | tccatggtgc tggatcataa catcattcct ggcctgtcca tcctgaatct gattttcttt |
| | atcctgtgca cattcgtgca gctgctggga ggctggtact tttatgtgca ggcatataaa |
| | tcactgcgac accggagcgc caatatggac gtgctgattg tcctggcaac ctctatcgcc |
| | tacgtgtata gtctggtcat cctggtggtc gcagtggcag agaaggcaga acggagcccc |
| | gtgactttct ttgatacccc tccaatgctg ttcgtgttta tcgctctggg cagatggctg |
| | gaacatctgg caaagtcaaa aaccagcgag gctctggcaa agctgatgag cctgcaggct |
| | accgaagcaa cagtggtcac tctgggagag gacaacctga tcattcgcga ggaacaggtg |
| | cctatggaac tggtccagcg aggcgacatc gtgaaggtgg tcccaggggg aaaattcccc |
| | gtggacggca aggtcctgga ggggaatact atggccgatg aatccctgat caccggcgag |
| | gctatgcctg tgacaaagaa accaggatca actgtcattg ctggcagcat caacgcacac |
| | gggtccgtgc tgatcaaggc cacacatgtc gggaatgaca caactctggc tcagattgtg |
| | aaactggtcg aggaagccca gatgtccaag gctcctatcc agcagctggc cgatcggttc |
| | tccggctact tcgtgccctt catcattatc atgtctacac tgactctggt ggtctggatt |
| | gtgatcggat tcattgactt tggcgtggtc cagagatatt ttcccaaccc taataagcac |
| | atcagccaga ccgaagtgat catcaggttc gcatttcaga ccagtattac agtgctgtgc |
| | atcgcctgcc catgttcact ggggctggct acccccacag cagtgatggt cggaacaggg |
| | gtggcagcac agaacggcat cctgatcaag ggcgggaaac ccctggagat ggcccacaag |
| | atcaaaactg tgatgtttga caaaactggg accattacac atggagtgcc acgcgtcatg |
| | cgagtgctgc tgctgggcga tgtggcaacc ctgcctctga gaaaggtcct ggcagtggtc |
| | ggaacagcag aggctagctc cgaacaccca ctgggggtgg ccgtcacaaa gtactgcaaa |
| | gaggaactgg gcactgagac cctggggtat tgtactgact tccaggcagt gccaggatgc |
| | ggaatcggat gtaaagtctc taacgtggaa gggattctgg ctcacagtga gcggcccctg |
| | agcgcacctg catcccatct gaatgaagca ggaagcctgc cagcagagaa ggacgctgtg |
| | cctcagacct tttccgtcct gatcggcaac agagaatggc tgcggagaaa tgggctgaca |
| | atttctagtg acgtgtccga tgccatgaca gatcacgaga tgaaaggcca gactgcaatt |
| | ctggtggcca tcgacggagt cctgtgcggc atgattgcta tcgcagatgc cgtgaagcag |
| | gaggctgcac tggccgtcca taccctgcag tctatgggcg tggacgtggt cctgatcacc |
| | ggggataacc ggaaaacagc tagagcaatt gccactcaag tgggcatcaa taaggtgttc |
| | gctgaagtcc tgcctagcca caaggtcgca aaagtgcagg agctgcagaa caagggcaag |
| | aaagtcgcca tggtgggaga cggcgtgaat gatagcccag ctctggcaca ggcagacatg |
| | ggagtcgcta ttgggacagg aactgacgtg gcaatcgagg ccgctgatgt ggtcctgatt |
| | aggaatgacc tgctggatgt ggtcgcttct attcatctga gtaagaggac agtgaggcgc |
| | attcgcatca acctggtgct ggccctgatc tacaatctgg tgggcatccc catcgcagca |
| | ggcgtgttta tgccaattgg gatcgtcctg cagccctgga tgggctcagc tgcaatggcc |
| | gcttcaagcg tgagcgtggt cctgtcctct ctgcagctga aatgctacaa gaaaccagat |
| | ctggagcggt acgaagctca ggcacacgga catatgaagc ccctgaccgc ttcccaggtg |
| | tctgtccaca tcggcatgga cgatagatgg agggacagcc caagggcaac tccatgggat |
| | caggtcagtt acgtgagcca ggtcagcctg agttcactga ccagcgacaa gccctcccgc |
| | cattctgcag ccgctgatga cgacggggac aagtggagcc tgctgctgaa cgggagggac |
| | gaagaacagt acatttga |
| 31 | taggtcttgaaaggagtgggaattggctccggtgcccgtcagtgggcagagcgcacatcgcccacagtccccgagaagttggg |
| | gggaggggtcggcaattgatccggtgcctagagaaggtggcgcggggtaaactgggaaagtgatgtcgtgtactggctccgcct |
| | ttttcccgagggtgggggagaaccgtatataagtgcagtagtcgccgtgaacgttctttttcgcaacgggtttgccgccagaacaca |
| | ggaccggttctagagcgctgccaccatggagaagaggctgggagtcaagccaaatcctgcttcctggattttatcaggatattatt |
| | ggcagacatctgcgaagtggttgagaagcctgtacctgttttatacttgcttttgcttcagcgttctgtggttgtcaacagatgccagtg |
| | agagcaggtgccagcaggggaagacacaatttggagttggcctgagatctgggggagaaaatcacctctggcttcttgaagga |
| | accccctctctccagtcatgttgggctgcctgctgccaggactctgcctgccatgtcttttggtggctagaagggatgtgcattcaggc |
| | agactgcagcaggccccagagctgccgggcttttaggacacactcctccaattccatgctggtgtttttaaaaaaattccaaactgc |
| | agatgatttgggctttctacctgaagatgatgtaccacatcttctggggctaggttggaactgggcatcttggaggcagagcccacc |
| | cagagctgcactcagacctgctgtatcttccagtgaccagcagagcttaatcaggaagcttcagaagagaggtagtcccagtga |
| | cgtagttacacctatagtgacacagcattctaaagtgaatgactccaacgaattaggtggtctgactaccagtggctctgcagagg |
| | tccacaaggcgattacaatttccagtcccctaaccacagacctgactgcagagctgtctggtgggccaaagaatgtatcagtgca |
| | acctgaaatatcagagggtcttgctactacgcccagcactcaacaagtaaaaagttctgagaaaacccagattgctgtccccca |
| | gccagtggctccctcctacagttatgctacccctaccccccaggcctctttccagagcacctcagcaccatacccagttataaagg |
| | aactggtggtatctgctggagagagtgtccagataaccctgcctaagaatgaagttcaattaaatgcatatgttctccaagaacca |
| | cctaaaggagaaacctacacctacgactggcagctgattactcatcctagagactacagtggagaaatggaagggaaacattc |
| | ccagatcctcaaactatcgaagctcactccaggcctgtatgaattcaaagtgattgtagagggtcaaaatgcccatggggaaggc |
| | tatgtgaacgtgacagtcaagccagagccccgtaagaatcggccccccattgctattgtgtcacctcagttccaggagatctctttg |
| | ccaaccacttctacagtcattgatggcagtcaaagcactgatgatgataaaatcgttcagtaccattgggaagaacttaaggggc |
| | ctctaagagaagagaagatttctgaagatacagccatattaaaactaagtaaactcgtccctgggaactacactttcagcttgact |
| | gtagtagactctgatggagctaccaactctactactgcaaacctgacagtgaacaaagctgtggattacccccctgtggccaacg |
| | caggccccaaccaagtgatcaccctgccccaaaactccatcaccctctttgggaaccagagcactgatgatcatggcatcacca |
| | gctatgagtggtcactcagcccaagcagcaaagggaaagtggtggagatgcagggtgttagaacaccaaccttacagctctct |
| | gcgatgcaagaaggagactacacttaccagctcacagtgactgacacaataggacagcaggccactgctcaagtgactgttatt |
| | gtgcaacctgaaaacaataagcctcctcaggcagatgcaggcccagataaagagctgacccttcctgtggatagcacaaccct |
| | ggatggcagcaagagctcagatgatcagaaaattatctcatatctctgggaaaaaacacagggacctgatggggtgcagctcg |
| | agaatgctaacagcagtgttgctactgtgactgggctgcaagtggggacctatgtgttcaccttgactgtcaaagatgagaggaac |
| | ctgcaaagccagagctctgtgaatgtcattgtcaaagaagaaataaacaaaccacctatagccaagataactgggaatgtggtg |
| | attaccctacccacgagcacagcagagctggatggctctaagtcctcagatgacaagggaatagtcagctacctctggactcga |
| | gatgaggggagcccagcagcaggggaggtgttaaatcactctgaccatcaccctatcctttttctttcaaacctggttgagggaac |
| | ctacacttttcacctgaaagtgaccgatgcaaagggtgagagtgacacagaccggaccactgtggaggtgaaacctgatccca |
| | ggaaaaacaacctggtggagatcatcttggatatcaacgtcagtcagctaactgagaggctgaaggggatgttcatccgccaga |
| | ttggggtcctcctgggggtgctggattccgacatcattgtgcaaaagattcagccgtacacggagcagagcaccaaaatggtattt |
| | tttgttcaaaacgagcctccccaccagatcttcaaaggccatgaggtggcagcgatgctcaagagtgagctgcggaagcaaaa |
| | ggcagactttttgatattcagagccttggaagtcaacactgtcacatgtcagctgaactgttccgaccatggccactgtgactcgttc |
| | accaaacgctgtatctgtgaccctttttggatggagaatttcatcaaggtgcagctgagggatggagacagcaactgtgagtggag |
| | cgtgttatatgttatcattgctacctttgtcattgttgttgccttgggaatcctgtcttggactgtgatctgttgttgtaagaggcaaaaagg |
| | aaaacccaagaggaaaagcaagtacaagatcctggatgccacggatcaggaaagcctggagctgaagccaacctcccgag |
| | caggcatcaaacagaaaggccttttgctaagtagcagcctgatgcactccgagtcagagctggacagcgatgatgccatctttac |
| | atggccagaccgagagaagggcaaactcctgcatggtcagaatggctctgtacccaacgggcagacccctctgaaggccag |
| | gagcccgcgggaggagatcctgtagGATTACAAAGACGATGACGATAAGGGATCCGGCGCAACAA |
| | ACTTCTCTCTGCTGAAACAAGCCGGAGATGTCGAAGAGAATCCTGGACCGACCGAGTA |
| | CAAGCCCACGGTGCGCCTCGCCACCCGCGACGACGTCCCCAGGGCCGTACGCACCCT |
| | CGCCGCCGCGTTCGCCGACTACCCCGCCACGCGCCACACCGTCGATCCGGACCGCCA |
| | CATCGAGCGGGTCACCGAGCTGCAAGAACTCTTCCTCACGCGCGTCGGGCTCGACATC |
| | GGCAAGGTGTGGGTCGCGGACGACGGCGCCGCGGTGGCGGTCTGGACCACGCCGGA |
| | GAGCGTCGAAGCGGGGGCGGTGTTCGCCGAGATCGGCCCGCGCATGGCCGAGTTGA |
| | GCGGTTCCCGGCTGGCCGCGCAGCAACAGATGGAAGGCCTCCTGGCGCCGCACCGG |
| | CCCAAGGAGCCCGCGTGGTTCCTGGCCACCGTCGGAGTCTCGCCCGACCACCAGGGC |
| | AAGGGTCTGGGCAGCGCCGTCGTGCTCCCCGGAGTGGAGGCGGCCGAGCGCGCCGG |
| | GGTGCCCGCCTTCCTGGAGACCTCCGCGCCCCGCAACCTCCCCTTCTACGAGCGGCT |
| | CGGCTTCACCGTCACCGCCGACGTCGAGGTGCCCGAAGGACCGCGCACCTGGTGCAT |
| | GACCCGCAAGCCCGGTGCCTGA |
| 32 | TAGAGGGTATATAATGGAAGCTCGACTTCCAG |
| 33 | TGCRCNC |
| 34 | MAPKKKRKVGIHGVPAALEPGEKPYKCPECGKSFSRSDNLVRHQRTHTGEKPYKCPECGK |
| | SFSREDNLHTHQRTHTGEKPYKCPECGKSFSRSDELVRHQRTHTGEKPYKCPECGKSFSQ |
| | SGNLTEHQRTHTGEKPYKCPECGKSFSTSGHLVRHQRTHTGEKPYKCPECGKSFSQNSTL |
| | TEHQRTHTGKKTSKRPAATKKAGQAKKKKGSYPYDVPDYALEDALDDFDLDMLGSDALDD |
| | FDLDMLGSDALDDFDLDMLGSDALDDFDLDML |
| 35 | MAPKKKRKVGIHGVPA ALEPGEKPYKCPECGK SFSRSDNLVRHQRTHT |
| | GEKPYKCPECGKSFSH RTTLTNHQRTHTGEKP YKCPECGKSFSREDNL |
| | HTHQRTHTGEKPYKCP ECGKSFSTSHSLTEHQ RTHTGEKPYKCPECGK |
| | SFSQSSSLVRHQRTHT GEKPYKCPECGKSFSR EDNLHTHQRTHTGKKT |
| | SKRPAATKKAGQAKKK KGSYPYDVPDYALEEA SGSGRADALDDFDLDM |
| | LGSDALDDFDLDMLGS DALDDFDLDMLGSDAL DDFDLDMLINSRSSGS |
| | PKKKRKVGSQYLPDTD DRHRIEEKRKRTYETF KSIMKKSPFSGPTDPR |
| | PPPRRIAVPSRSSASV PKPAPQPYPFTSSLST INYDEFPTMVFPSGQI |
| | SQASALAPAPPQVLPQ APAPAPAPAMVSALAQ APAPVPVLAPGPPQAV |
| | APPAPKPTQAGEGTLS EALLQLQFDDEDLGAL LGNSTDPAVFTDLASV |
| | DNSEFQQLLNQGIPVA PHTTEPMLMEYPEAIT RLVTGAQRPPDPAPAP |
| | LGAPGLPNGLLSGDED FSSIADMDFSALLGSG SGSRDSREGMFLPKPE |
| | AGSAISDVFEGREVCQ PKRIRPFHPPGSPWAN RPLPASLAPTPTGPVH |
| | EPVGSLTPAPVPQPLD PAPAVTPEASHLLEDP DEETSQAVKALREMAD |
| | TVIPQKEEAAICGQMD LSHPPPRGHLDELTTT LESMTEDLNLDSPLTP |
| | ELNEILDTFLNDECLL HAMHISTGLSIFDTSL F |
| 36 | MAPKKKRKVGIHGVPA ALEPGEKPYKCPECGK SFSRSDNLVRHQRTHT |
| | GEKPYKCPECGKSFSR EDNLHTHQRTHTGEKP YKCPECGKSFSRSDEL |
| | VRHQRTHTGEKPYKCP ECGKSFSQSGNLTEHQ RTHTGEKPYKCPECGK |
| | SFSTSGHLVRHQRTHT GEKPYKCPECGKSFSQ NSTLTEHQRTHTGKKT |
| | SKRPAATKKAGQAKKK KGSYPYDVPDYALEEA SGSGRADALDDFDLDM |
| | LGSDALDDFDLDMLGS DALDDFDLDMLGSDAL DDFDLDMLINSRSSGS |
| | PKKKRKVGSQYLPDTD DRHRIEEKRKRTYETF KSIMKKSPFSGPTDPR |
| | PPPRRIAVPSRSSASV PKPAPQPYPFTSSLST INYDEFPTMVFPSGQI |
| | SQASALAPAPPQVLPQ APAPAPAPAMVSALAQ APAPVPVLAPGPPQAV |
| | APPAPKPTQAGEGTLS EALLQLQFDDEDLGAL LGNSTDPAVFTDLASV |
| | DNSEFQQLLNQGIPVA PHTTEPMLMEYPEAIT RLVTGAQRPPDPAPAP |
| | LGAPGLPNGLLSGDED FSSIADMDFSALLGSG SGSRDSREGMFLPKPE |
| | AGSAISDVFEGREVCQ PKRIRPFHPPGSPWAN RPLPASLAPTPTGPVH |
| | EPVGSLTPAPVPQPLD PAPAVTPEASHLLEDP DEETSQAVKALREMAD |
| | TVIPQKEEAAICGQMD LSHPPPRGHLDELTTT LESMTEDLNLDSPLTP |
| | ELNEILDTFLNDECLL HAMHISTGLSIFDTSL F |
| 37 | MAPKKKRKVGIHGVPA ALEPGEKPYKCPECGK SFSRSDNLVRHQRTHT |
| | GEKPYKCPECGKSFSH RTTLTNHQRTHTGEKP YKCPECGKSFSREDNL |
| | HTHQRTHTGEKPYKCP ECGKSFSTSHSLTEHQ RTHTGEKPYKCPECGK |
| | SFSQSSSLVRHQRTHT GEKPYKCPECGKSFSR EDNLHTHQRTHTGKKT |
| | SKRPAATKKAGQAKKK KGSYPYDVPDYALEDA LDDFDLDMLGSDALDD |
| | FDLDMLGSDALDDFDL DMLGSDALDDFDLDML |
| 38 | MAPKKKRKVGIHGVPA ALEPGEKPYKCPECGK SFSRSDNLVRHQRTHT |
| | GEKPYKCPECGKSFSR EDNLHTHQRTHTGEKP YKCPECGKSFSRSDEL |
| | VRHQRTHTGEKPYKCP ECGKSFSQSGNLTEHQ RTHTGEKPYKCPECGK |
| | SFSTSGHLVRHQRTHT GEKPYKCPECGKSFSQ NSTLTEHQRTHTGKKT |
| | SKRPAATKKAGQAKKK KGSYPYDVPDYALEDA LDDFDLDMLGSDALDD |
| | FDLDMLGSDALDDFDL DMLGSDALDDFDLDML |
| 39 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GQSQLIKPSRMRKYPN RPSKTPPHERPYACPV ESCDRRFSRSDNLVRH |
| | IRIHTGQKPFQCRICM RNFSHRTTLTNHIRTH TGEKPFACDICGRKFA |
| | REDNLHTHTKIHLRQK DRPYACPVESCDRRFS TSHSLTEHIRIHTGQK |
| | PFQCRICMRNFSQSSS LVRHIRTHTGEKPFAC DICGRKFAREDNLHTH |
| | TKIHLRQKDKLEMADH LMLAEGYRLVQRPPSA AAAHGPHALRTLPPYA |
| | GPGLDSGLRPRGAPLG PPPPRQPGALAYGAFG PPSSFQPFPAVPPPAA |
| | GIAHLQPVATPYPGRA AAPPNAPGGPPGPQPA PSAAAPPPPAHALGGM |
| | DAELIDEEALTSLELE LGLHRVRELPELFLGQ SEFDCFSDLGSAPPAG |
| | SVSC |
| 40 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GQSQLIKPSRMRKYPN RPSKTPPHERPYACPV ESCDRRFSRSDNLVRH |
| | IRIHTGQKPFQCRICM RNFSHRTTLTNHIRTH TGEKPFACDICGRKFA |
| | REDNLHTHTKIHLRQK DRPYACPVESCDRRFS TSHSLTEHIRIHTGQK |
| | PFQCRICMRNFSQSSS LVRHIRTHTGEKPFAC DICGRKFAREDNLHTH |
| | TKIHLRQKDK |
| 41 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GQSQLIKPSRMRKYPN RPSKTPPHERPYACPV ESCDRRFSRSDNLVRH |
| | IRIHTGQKPFQCRICM RNFSREDNLHTHIRTH TGEKPFACDICGRKFA |
| | RSDELVRHTKIHLRQK DRPYACPVESCDRRFS QSGNLTEHIRIHTGQK |
| | PFQCRICMRNFSTSGH LVRHIRTHTGEKPFAC DICGRKFAQNSTLTEH |
| | TKIHLRQKDKLEMADH LMLAEGYRLVQRPPSA AAAHGPHALRTLPPYA |
| | GPGLDSGLRPRGAPLG PPPPRQPGALAYGAFG PPSSFQPFPAVPPPAA |
| | GIAHLQPVATPYPGRA AAPPNAPGGPPGPQPA PSAAAPPPPAHALGGM |
| | DAELIDEEALTSLELE LGLHRVRELPELFLGQ SEFDCFSDLGSAPPAG |
| | SVSC |
| 42 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GQSQLIKPSRMRKYPN RPSKTPPHERPYACPV ESCDRRFSRSDNLVRH |
| | IRIHTGQKPFQCRICM RNFSREDNLHTHIRTH TGEKPFACDICGRKFA |
| | RSDELVRHTKIHLRQK DRPYACPVESCDRRFS QSGNLTEHIRIHTGQK |
| | PFQCRICMRNFSTSGH LVRHIRTHTGEKPFAC DICGRKFAQNSTLTEH |
| | TKIHLRQKDK |
| 43 | MQSQLIKPSRMRKYPN RPSKTPPHERPYACPV ESCDRRFSRSDNLVRH |
| | IRIHTGQKPFQCRICM RNFSREDNLHTHIRTH TGEKPFACDICGRKFA |
| | RSDELVRHTKIHLRQK DRPYACPVESCDRRFS QSGNLTEHIRIHTGQK |
| | PFQCRICMRNFSTSGH LVRHIRTHTGEKPFAC DICGRKFAQNSTLTEH |
| | TKIHLRQKDKLEMADH LMLAEGYRLVQRPPSA AAAHGPHALRTLPPYA |
| | GPGLDSGLRPRGAPLG PPPPRQPGALAYGAFG PPSSFQPFPAVPPPAA |
| | GIAHLQPVATPYPGRA AAPPNAPGGPPGPQPA PSAAAPPPPAHALGGM |
| | DAELIDEEALTSLELE LGLHRVRELPELFLGQ SEFDCFSDLGSAPPAG |
| | SVSC |
| 44 | MQSQLIKPSRMRKYPN RPSKTPPHERPYACPV ESCDRRFSRSDNLVRH |
| | IRIHTGQKPFQCRICM RNFSREDNLHTHIRTH TGEKPFACDICGRKFA |
| | RSDELVRHTKIHLRQK DRPYACPVESCDRRFS QSGNLTEHIRIHTGQK |
| | PFQCRICMRNFSTSGH LVRHIRTHTGEKPFAC DICGRKFAQNSTLTEH |
| | TKIHLRQKDKLEMSGL EMADHMMAMNHGRFPD GTNGLHHHPAHRMGMG |
| | QFPSPHHHQQQQPQHA FNALMGEHIHYGAGNM NATSGIRHAMGPGTVN |
| | GGHPPSALAPAARFNN SQFMGPPVASQGGSLP ASMQLQKLNNQYFNHH |
| | PYPHNHYMPDLHPAAG HQMNGTNQHFRDCNPK HSGGSSTPGGSGGSST |
| | PGGSGSSSGGGAGSSN SGGGSGSGNMPASVAH VPAAMLPPNVIDTDFI |
| | DEEVLMSLVIEMGLDR IKELPELWLGQNEFDF MTDFVCKQQPSRVSC |
| 45 | MSGLEMADHMMAMNHG RFPDGTNGLHHHPAHR MGMGQFPSPHHHQQQQ |
| | PQHAFNALMGEHIHYG AGNMNATSGVRHAMGP GTVNGGHPPSALAPAA |
| | RFNNSQFMGPPVASQG GSLPASMQLQKLNNQY FNHHPYPHNHYMPDLH |
| | PAAGHQMNGTNQHFRD CNPKHSGGSSTPGGSG GSSTPGGSGSSSGGGA |
| | GSSNSGGGSGSGNMPA SVAHVPAAMLPPNVID TDFIDEEVLMSLVIEM |
| | GLDRIKELPELWLGQN EFDFMTDFVCKQQPSR VSCQSQLIKPSRMRKY |
| | PNRPSKTPPHERPYAC PVESCDRRFSRSDNLV RHIRIHTGQKPFQCRI |
| | CMRNFSREDNLHTHIR THTGEKPFACDICGRK FARSDELVRHTKIHLR |
| | QKDRPYACPVESCDRR FSQSGNLTEHIRIHTG QKPFQCRICMRNFSTS |
| | GHLVRHIRTHTGEKPF ACDICGRKFAQNSTLT EHTKIHLRQKDK |
| 46 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GRPHACPAEGCDRRFS RSDNLVRHLRIHTGHK PFQCRICMRSFSREDN |
| | LHTHIRTHTGEKPFAC EFCGRKFARSDELVRH AKIHLKQKEHACPAEG |
| | CDRRFSQSGNLTEHLR IHTGHKPFQCRICMRS FSTSGHLVRHIRTHTG |
| | EKPFACEFCGRKFAQN STLTEHAKIHLKQKEK LEMADHLMLAEGYRLV |
| | QRPPSAAAAHGPHALR TLPPYAGPGLDSGLRP RGAPLGPPPPRQPGAL |
| | AYGAFGPPSSFQPFPA VPPPAAGIAHLQPVAT PYPGRAAAPPNAPGGP |
| | PGPQPAPSAAAPPPPA HALGGMDAELIDEEAL TSLELELGLHRVRELP |
| | ELFLGQSEFDCFSDLG SAPPAGSVSC |
| 47 | MRPHACPAEGCDRRFS RSDNLVRHLRIHTGHK PFQCRICMRSFSREDN |
| | LHTHIRTHTGEKPFAC EFCGRKFARSDELVRH AKIHLKQKEHACPAEG |
| | CDRRFSQSGNLTEHLR IHTGHKPFQCRICMRS FSTSGHLVRHIRTHTG |
| | EKPFACEFCGRKFAQN STLTEHAKIHLKQKEK LEMADHLMLAEGYRLV |
| | QRPPSAAAAHGPHALR TLPPYAGPGLDSGLRP RGAPLGPPPPRQPGAL |
| | AYGAFGPPSSFQPFPA VPPPAAGIAHLQPVAT PYPGRAAAPPNAPGGP |
| | PGPQPAPSAAAPPPPA HALGGMDAELIDEEAL TSLELELGLHRVRELP |
| | ELFLGQSEFDCFSDLG SAPPAGSVSC |
| 48 | MRPHACPAEGCDRRFS RSDNLVRHLRIHTGHK PFQCRICMRSFSREDN |
| | LHTHIRTHTGEKPFAC EFCGRKFARSDELVRH AKIHLKQKEHACPAEG |
| | CDRRFSQSGNLTEHLR IHTGHKPFQCRICMRS FSTSGHLVRHIRTHTG |
| | EKPFACEFCGRKFAQN STLTEHAKIHLKQKEK KAEKGGAPSASSAPPV |
| | SLAPVVTTCALEMSGL EMADHMMAMNHGRFPD GTNGLHHHPAHRMGMG |
| | QFPSPHHHQQQQPQHA FNALMGEHIHYGAGNM NATSGIRHAMGPGTVN |
| | GGHPPSALAPAARFNN SQFMGPPVASQGGSLP ASMQLQKLNNQYFNHH |
| | PYPHNHYMPDLHPAAG HQMNGTNQHFRDCNPK HSGGSSTPGGSGGSST |
| | PGGSGSSSGGGAGSSN SGGGSGSGNMPASVAH VPAAMLPPNVIDTDFI |
| | DEEVLMSLVIEMGLDR IKELPELWLGQNEFDF MTDFVCKQQPSRVSC |
| 49 | MSGLEMADHMMAMNHG RFPDGTNGLHHHPAHR MGMGQFPSPHHHQQQQ |
| | PQHAFNALMGEHIHYG AGNMNATSGVRHAMGP GTVNGGHPPSALAPAA |
| | RFNNSQFMGPPVASQG GSLPASMQLQKLNNQY FNHHPYPHNHYMPDLH |
| | PAAGHQMNGTNQHFRD CNPKHSGGSSTPGGSG GSSTPGGSGSSSGGGA |
| | GSSNSGGGSGSGNMPA SVAHVPAAMLPPNVID TDFIDEEVLMSLVIEM |
| | GLDRIKELPELWLGQN EFDFMTDFVCKQQPSR VSCRPHACPAEGCDRR |
| | FSRSDNLVRHLRIHTG HKPFQCRICMRSFSRE DNLHTHIRTHTGEKPF |
| | ACEFCGRKFARSDELV RHAKIHLKQKEHACPA EGCDRRFSQSGNLTEH |
| | LRIHTGHKPFQCRICM RSFSTSGHLVRHIRTH TGEKPFACEFCGRKFA |
| | QNSTLTEHAKIHLKQK EKKAEKGGAPSASSAP PVSLAPVVTTCA |
| 50 | MTGKLAEKLPVTMSSL LNQLPDNLYPEEIPSA LNLFSGSSDSVVHYNQ |
| | MATENVMDIGLTNEKP NPELSYSGSFQPAPGN KTVTYLGKFAFDSPSN |
| | WCQDNIISLMSAGILG VPPASGALSTQTSTAS MVQPPQGDVEAMYPAL |
| | PPYSNCGDLYSEPVSF HDPQGNPGLAYSPQDY QSAKPALDSNLFPMIP |
| | DYNLYHHPNDMGSIPE HKPFQGMDPIRVNPPP ITPLETIKAFKDKQIH |
| | PGFGSLPQPPLTLKPI RPRKYPNRPSKTPLHE RPHACPAEGCDRRFSR |
| | RDELNVHLRIHTGHKP FQCRICMRSFSRSDHL TNHIRTHTGEKPFACE |
| | FCGRKFARSDDLVRHA KIHLKQKEHACPAEGC DRRFSRSDNLVRHLRI |
| | HTGHKPFQCRICMRSF SHRTTLTNHIRTHTGE KPFACEFCGRKFARED |
| | NLHTHAKIHLKQKEHA CPAEGCDRRFSTSHSL TEHLRIHTGHKPFQCR |
| | ICMRSFSQSSSLVRHI RTHTGEKPFACEFCGR KFAREDNLHTHAKIHL |
| | KQKEKKAEKGGAPSAS SAPPVSLAPVVTTCA |
| 51 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLVRHIRIHTGQKPF QCRICMRNFSHRTTLT |
| | NHIRTHTGEKPFACDI CGRKFAREDNLHTHTK IHLRQKDRPYACPVES |
| | CDRRFSTSHSLTEHIR IHTGQKPFQCRICMRN FSQSSSLVRHIRTHTG |
| | EKPFACDICGRKFARE DNLHTHTKIHLRQKDK KADKSVVASSATSSLS |
| | SYPSPVATSYPSPVTT SYPSPATTSYPSPVPT SFSSPGSSTYPSPVHS |
| | GFPSPSVATTYSSVPP AFPAQVSSFPSSAVTN SFSASTGLSDMTATFS |
| | PRTIEIC |
| 52 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRR DELNVHIRIHTGQKPF QCRICMRNFSRSDHLT |
| | NHIRTHTGEKPFACDI CGRKFARSDDLVRHTK IHLROKDRPYACPVES |
| | CDRRFSRSDNLVRHIR IHTGQKPFQCRICMRN FSHRTTLTNHIRTHTG |
| | EKPFACDICGRKFARE DNLHTHTKIHLROKDR PYACPVESCDRRFSTS |
| | HSLTEHIRIHTGQKPF QCRICMRNFSQSSSLV RHIRTHTGEKPFACDI |
| | CGRKFAREDNLHTHTK IHLRQKDKKADKSVVA SSATSSLSSYPSPVAT |
| | SYPSPVTTSYPSPATT SYPSPVPTSFSSPGSS TYPSPVHSGFPSPSVA |
| | TTYSSVPPAFPAQVSS FPSSAVTNSFSASTGL SDMTATFSPRTIEIC |
| 53 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLVRHIRIHTGQKPF QCRICMRNFSHRTTLT |
| | NHIRTHTGEKPFACDI CGRKFAREDNLHTHIR THTGEKPFACDICGRK |
| | FSTSHSLTEHIRIHTG QKPFQCRICMRNFSQS SSLVRHIRTHTGEKPF |
| | ACDICGRKFAREDNLH THTKIHLRQKDKKADK SVVASSATSSLSSYPS |
| | PVATSYPSPVTTSYPS PATTSYPSPVPTSFSS PGSSTYPSPVHSGFPS |
| | PSVATTYSSVPPAFPA QVSSFPSSAVTNSFSA STGLSDMTATFSPRTI |
| | EIC |
| 54 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLVRHIRIHTGQKPF QCRICMRNFSREDNLH |
| | THIRTHTGEKPFACDI CGRKFARSDELVRHTK IHLRQKDRPYACPVES |
| | CDRRFSQSGNLTEHIR IHTGQKPFQCRICMRN FSTSGHLVRHIRTHTG |
| | EKPFACDICGRKFAQN STLTEHTKIHLRQKDK KADKSVVASSATSSLS |
| | SYPSPVATSYPSPVTT SYPSPATTSYPSPVPT SFSSPGSSTYPSPVHS |
| | GFPSPSVATTYSSVPP AFPAQVSSFPSSAVTN SFSASTGLSDMTATFS |
| | PRTIEIC |
| 55 | MTGKLAEKLPVTMSSL LNQLPDNLYPEEIPSA LNLFSGSSDSVVHYNQ |
| | MATENVMDIGLTNEKP NPELSYSGSFQPAPGN KTVTYLGKFAFDSPSN |
| | WCQDNIISLMSAGILG VPPASGALSTQTSTAS MVQPPQGDVEAMYPAL |
| | PPYSNCGDLYSEPVSF HDPQGNPGLAYSPQDY QSAKPALDSNLFPMIP |
| | DYNLYHHPNDMGSIPE HKPFQGMDPIRVNPPP ITPLETIKAFKDKQIH |
| | PGFGSLPQPPLTLKPI RPRKYPNRPSKTPLHE RPHACPAEGCDRRFSR |
| | SDNLVRHLRIHTGHKP FQCRICMRSFSHRTTL TNHIRTHTGEKPFACE |
| | FCGRKFAREDNLHTHA KIHLKQKEHACPAEGC DRRFSTSHSLTEHLRI |
| | HTGHKPFQCRICMRSF SQSSSLVRHIRTHTGE KPFACEFCGRKFARED |
| | NLHTHAKIHLKQKEKK AEKGGAPSASSAPPVS LAPVVTTCA |
| 56 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSDP GALVRHIRIHTGQKPF QCRICMRNFSRSDNLV |
| | RHIRTHTGEKPFACDI CGRKFAQSGDLRRHTK IHLRQKDRPYACPVES |
| | CDRRFSTHLDLIRHIR IHTGQKPFQCRICMRN FSTSGNLVRHIRTHTG |
| | EKPFACDICGRKFARS DNLVRHTKIHLRQKDR PYACPVESCDRRFSQS |
| | GHLTEHIRIHTGQKPF QCRICMRNFSERSHLR EHIRTHTGEKPFACDI |
| | CGRKFAQAGHLASHTK IHLROKDKKADKSVVA SSATSSLSSYPSPVAT |
| | SYPSPVTTSYPSPATT SYPSPVPTSFSSPGSS TYPSPVHSGFPSPSVA |
| | TTYSSVPPAFPAQVSS FPSSAVTNSFSASTGL SDMTATFSPRTIEIC |
| 57 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLTRHIRIHTGQKPF QCRICMRNFSHSTTLT |
| | NHIRTHTGEKPFACDI CGRKFARSDNRKTHIR THTGEKPFACDICGRK |
| | FSTSHSLTEHIRIHTG QKPFQCRICMRNFSQS SSLTRHIRTHTGEKPF |
| | ACDICGRKFARSDNRK THTKIHLRQKDKKADK SVVASSATSSLSSYPS |
| | PVATSYPSPVTTSYPS PATTSYPSPVPTSFSS PGSSTYPSPVHSGFPS |
| | PSVATTYSSVPPAFPA QVSSFPSSAVTNSFSA STGLSDMTATFSPRTI |
| | EIC |
| 58 | MTGKLAEKLPVTMSSL LNQLPDNLYPEEIPSA LNLFSGSSDSVVHYNQ |
| | MATENVMDIGLTNEKP NPELSYSGSFQPAPGN KTVTYLGKFAFDSPSN |
| | WCQDNIISLMSAGILG VPPASGALSTQTSTAS MVQPPQGDVEAMYPAL |
| | PPYSNCGDLYSEPVSF HDPQGNPGLAYSPQDY QSAKPALDSNLFPMIP |
| | DYNLYHHPNDMGSIPE HKPFQGMDPIRVNPPP ITPLETIKAFKDKQIH |
| | PGFGSLPQPPLTLKPI RPRKYPNRPSKTPLHE RPHACPAEGCDRRFSR |
| | SDNLVRHLRIHTGHKP FQCRICMRSFSREDNL HTHIRTHTGEKPFACE |
| | FCGRKFARSDELVRHA KIHLKQKEHACPAEGC DRRFSQSGNLTEHLRI |
| | HTGHKPFQCRICMRSF STSGHLVRHIRTHTGE KPFACEFCGRKFAQNS |
| | TLTEHAKIHLKQKEKK AEKGGAPSASSAPPVS LAPVVTTCA |
| 59 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLTRHIRIHTGQKPF QCRICMRNFSRSDNLT |
| | THIRTHTGEKPFACDI CGRKFARSDERKRHIR THTGEKPFACDICGRK |
| | FSQSGNLTEHIRIHTG QKPFQCRICMRNFSTS GHLTRHIRTHTGEKPF |
| | ACDICGRKFAQSSTRK EHTKIHLROKDKKADK SVVASSATSSLSSYPS |
| | PVATSYPSPVTTSYPS PATTSYPSPVPTSFSS PGSSTYPSPVHSGFPS |
| | PSVATTYSSVPPAFPA QVSSFPSSAVTNSFSA STGLSDMTATFSPRTI |
| | EIC |
| 60 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DNLVRHIRIHTGQKPF QCRICMRNFSREDNLH |
| | THIRTHTGEKPFACDI CGRKFARSDELVRHIR THTGEKPFACDICGRK |
| | FSQSGNLTEHIRIHTG QKPFQCRICMRNFSTS GHLVRHIRTHTGEKPF |
| | ACDICGRKFAQNSTLT EHTKIHLRQKDKKADK SVVASSATSSLSSYPS |
| | PVATSYPSPVTTSYPS PATTSYPSPVPTSFSS PGSSTYPSPVHSGFPS |
| | PSVATTYSSVPPAFPA QVSSFPSSAVTNSFSA STGLSDMTATFSPRTI |
| | EIC |
| 61 | MAPKKKRKVGIHGVPA ALEPGEKPYKCPECGK SFSSPADLTRHQRTHT |
| | GEKPYKCPECGKSFSR SDNLVRHQRTHTGEKP YKCPECGKSFSREDNL |
| | HTHQRTHTGEKPYKCP ECGKSFSRSDELVRHQ RTHTGEKPYKCPECGK |
| | SFSQSGNLTEHQRTHT GEKPYKCPECGKSFST SGHLVRHQRTHTGKKT |
| | SKRPAATKKAGQAKKK KGSYPYDVPDYALEDA LDDFDLDMLGSDALDD |
| | FDLDMLGSDALDDFDL DMLGSDALDDFDLDML |
| 62 | MAPKKKRKVGIHGVPA ALEPGEKPYKCPECGK SFSDPGALVRHQRTHT |
| | GEKPYKCPECGKSFSR SDNLVRHQRTHTGEKP YKCPECGKSFSQSGDL |
| | RRHQRTHTGEKPYKCP ECGKSFSTHLDLIRHQ RTHTGEKPYKCPECGK |
| | SFSTSGNLVRHQRTHT GEKPYKCPECGKSFSR SDNLVRHQRTHTGKKT |
| | SKRPAATKKAGQAKKK KGSYPYDVPDYALEDA LDDFDLDMLGSDALDD |
| | FDLDMLGSDALDDFDL DMLGSDALDDFDLDML |
| 63 | MAPKKKRKVGIHGVPA ALEPGEKPYKCPECGK SFSRSDNLVRHQRTHT |
| | GEKPYKCPECGKSFSR EDNLHTHQRTHTGEKP YKCPECGKSFSRSDEL |
| | VRHQRTHTGEKPYKCP ECGKSFSQSGNLTEHQ RTHTGEKPYKCPECGK |
| | SFSTSGHLVRHQRTHT GEKPYKCPECGKSFSQ NSTLTEHQRTHTGKKT |
| | SKRPAATKKAGQAKKK KGSDALDDFDLDMLGS DALDDFDLDMLGSDAL |
| | DDFDLDMLGSDALDDF DLDML |
| 64 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCQSQLIKP |
| | SRMRKYPNRPSKTPPH ERPYACPVESCDRRFS RSDNLVRHIRIHTGQK |
| | PFQCRICMRNFSREDN LHTHIRTHTGEKPFAC DICGRKFARSDELVRH |
| | TKIHLRQKDRPYACPV ESCDRRFSQSGNLTEH IRIHTGQKPFQCRICM |
| | RNFSTSGHLVRHIRTH TGEKPFACDICGRKFA QNSTLTEHTKIHLRQK |
| | DK |
| 65 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GGGSGGGSGQSQLIKP SRMRKYPNRPSKTPPH ERPYACPVESCDRRFS |
| | RSDNLVRHIRIHTGQK PFQCRICMRNFSREDN LHTHIRTHTGEKPFAC |
| | DICGRKFARSDELVRH TKIHLRQKDRPYACPV ESCDRRFSQSGNLTEH |
| | IRIHTGQKPFQCRICM RNFSTSGHLVRHIRTH TGEKPFACDICGRKFA |
| | QNSTLTEHTKIHLRQK DK |
| 66 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCADHLMLA |
| | EGYRLVQRPPSAAAAH GPHALRTLPPYAGPGL DSGLRPRGAPLGPPPP |
| | RQPGALAYGAFGPPSS FQPFPAVPPPAAGIAH LQPVATPYPGRAAAPP |
| | NAPGGPPGPQPAPSAA APPPPAHALGGMDAEL IDEEALTSLELELGLH |
| | RVRELPELFLGQSEFD CFSDLGSAPPAGSVSC QSQLIKPSRMRKYPNR |
| | PSKTPPHERPYACPVE SCDRRFSRSDNLVRHI RIHTGQKPFQCRICMR |
| | NFSREDNLHTHIRTHT GEKPFACDICGRKFAR SDELVRHTKIHLRQKD |
| | RPYACPVESCDRRFSQ SGNLTEHIRIHTGQKP FQCRICMRNFSTSGHL |
| | VRHIRTHTGEKPFACD ICGRKFAQNSTLTEHT KIHLRQKDK |
| 67 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GADHLMLAEGYRLVQR PPSAAAAHGPHALRTL PPYAGPGLDSGLRPRG |
| | APLGPPPPRQPGALAY GAFGPPSSFQPFPAVP PPAAGIAHLQPVATPY |
| | PGRAAAPPNAPGGPPG PQPAPSAAAPPPPAHA LGGMDAELIDEEALTS |
| | LELELGLHRVRELPEL FLGQSEFDCFSDLGSA PPAGSVSCGGSGGGSG |
| | QSQLIKPSRMRKYPNR PSKTPPHERPYACPVE SCDRRFSRSDNLVRHI |
| | RIHTGQKPFQCRICMR NFSREDNLHTHIRTHT GEKPFACDICGRKFAR |
| | SDELVRHTKIHLRQKD RPYACPVESCDRRFSQ SGNLTEHIRIHTGQKP |
| | FQCRICMRNFSTSGHL VRHIRTHTGEKPFACD ICGRKFAQNSTLTEHT |
| | KIHLRQKDK |
| 68 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GGGSGGGSGQSQLIKP SRMRKYPNRPSKTPPH ERPYACPVESCDRRFS |
| | RSDNLVRHIRIHTGQK PFQCRICMRNFSREDN LHTHIRTHTGEKPFAC |
| | DICGRKFARSDELVRH TKIHLRQKDRPYACPV ESCDRRFSQSGNLTEH |
| | IRIHTGQKPFQCRICM RNFSTSGHLVRHIRTH TGEKPFACDICGRKFA |
| | QNSTLTEHTKIHLRQK DK |
| 69 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCADHLMLA |
| | EGYRLVQRPPSAAAAH GPHALRTLPPYAGPGL DSGLRPRGAPLGPPPP |
| | RQPGALAYGAFGPPSS FQPFPAVPPPAAGIAH LQPVATPYPGRAAAPP |
| | NAPGGPPGPQPAPSAA APPPPAHALGGMDAEL IDEEALTSLELELGLH |
| | RVRELPELFLGQSEFD CFSDLGSAPPAGSVSC QSQLIKPSRMRKYPNR |
| | PSKTPPHERPYACPVE SCDRRFSRSDNLVRHI RIHTGQKPFQCRICMR |
| | NFSREDNLHTHIRTHT GEKPFACDICGRKFAR SDELVRHTKIHLRQKD |
| | RPYACPVESCDRRFSQ SGNLTEHIRIHTGQKP FQCRICMRNFSTSGHL |
| | VRHIRTHTGEKPFACD ICGRKFAQNSTLTEHT KIHLRQKDK |
| 70 | MAADHLMLAEGYRLVQ RPPSAAAAHGPHALRT LPPYAGPGLDSGLRPR |
| | GAPLGPPPPRQPGALA YGAFGPPSSFQPFPAV PPPAAGIAHLQPVATP |
| | YPGRAAAPPNAPGGPP GPQPAPSAAAPPPPAH ALGGMDAELIDEEALT |
| | SLELELGLHRVRELPE LFLGQSEFDCFSDLGS APPAGSVSCGGSGGGS |
| | GADHLMLAEGYRLVQR PPSAAAAHGPHALRTL PPYAGPGLDSGLRPRG |
| | APLGPPPPRQPGALAY GAFGPPSSFQPFPAVP PPAAGIAHLQPVATPY |
| | PGRAAAPPNAPGGPPG PQPAPSAAAPPPPAHA LGGMDAELIDEEALTS |
| | LELELGLHRVRELPEL FLGQSEFDCFSDLGSA PPAGSVSCGGSGGGSG |
| | QSQLIKPSRMRKYPNR PSKTPPHERPYACPVE SCDRRFSRSDNLVRHI |
| | RIHTGQKPFQCRICMR NFSREDNLHTHIRTHT GEKPFACDICGRKFAR |
| | SDELVRHTKIHLRQKD RPYACPVESCDRRFSQ SGNLTEHIRIHTGQKP |
| | FQCRICMRNFSTSGHL VRHIRTHTGEKPFACD ICGRKFAQNSTLTEHT |
| | KIHLRQKDK |
| 71 | M E L E L D A G D Q D L L A F L |
| 72 | MELELDAGDQDLLAFL LEESGDLGTAPDEAVR APLDWALPLSEVPSDW |
| | EVDDLLCSLLSPPASL NILSSSNPCLVHHDHT YSLPRETVSMDLESES |
| | CRKEGTQMTPQHMEEL AEQEIARLVLTDEEKS LLEKEGLILPETLPLT |
| | KTEEQILKRVRRPYAC PVESCDRRFSRSDNLV RHIRIHTGQKPFQCRI |
| | CMRNFSREDNLHTHIR THTGEKPFACDICGRK FARSDELVRHTKIHLR |
| | QKDRPYACPVESCDRR FSQSGNLTEHIRIHTG QKPFQCRICMRNFSTS |
| | GHLVRHIRTHTGEKPF ACDICGRKFAQNSTLT EHTKIHLRQKDVYVGG |
| | LESRVLKYTAQNMELQ NKVQLLEEQNLSLLDQ LRKLQAMVIEISNKTS |
| | SSSTCILVLLVSFCLL LVPAMYSSDTRGSLPA EHGVLSRQLRALPSED |
| | PYQLELPALQSEVPKD STHQWLDGSDCVLQAP GNTSCLLHYMPQAPSA |
| | EPPLEWPFPDLFSEPL CRGPILPLQANLTRKG GWLPTGSPSVILQDRY |
| | SG |
| 73 | MELELDAGDQDLLAFL LEESGDLGTAPDEAVR APLDWALPLSEVPSDW |
| | EVDDLLCSLLSPPASL NILSSSNPCLVHHDHT YSLPRETVSMDLESES |
| | CRKEGTQMTPQHMEEL AEQEIARLVLTDEEKS LLEKEGLILPETLPLT |
| | KTEEQILKRVRRPYAC PVESCDRRFSRSDNLV RHIRIHTGQKPFQCRI |
| | CMRNFSHRTTLTNHIR THTGEKPFACDICGRK FAREDNLHTHTKIHLR |
| | QKDRPYACPVESCDRR FSTSHSLTEHIRIHTG QKPFQCRICMRNFSQS |
| | SSLVRHIRTHTGEKPF ACDICGRKFAREDNLH THTKIHLRQKDVYVGG |
| | LESRVLKYTAQNMELQ NKVQLLEEQNLSLLDQ LRKLQAMVIEISNKTS |
| | SSSTCILVLLVSFCLL LVPAMYSSDTRGSLPA EHGVLSRQLRALPSED |
| | PYQLELPALQSEVPKD STHQWLDGSDCVLQAP GNTSCLLHYMPQAPSA |
| | EPPLEWPFPDLFSEPL CRGPILPLQANLTRKG GWLPTGSPSVILQDRY |
| | SG |
| 74 | MELELDAGDQDLLAFL LEESGDLGTAPDEAVR APLDWALPLSEVPSDW |
| | EVDDLLCSLLSPPASL NILSSSNPCLVHHDHT YSLPRETVSMDLESES |
| | CRKEGTQMTPQHMEEL AEQEIARLVLTDEEKS LLEKEGLILPETLPLT |
| | KTEEQILKRVRLEPGE KPYKCPECGKSFSRSD NLVRHQRTHTGEKPYK |
| | CPECGKSFSREDNLHT HQRTHTGEKPYKCPEC GKSFSRSDELVRHQRT |
| | HTGEKPYKCPECGKSF SQSGNLTEHQRTHTGE KPYKCPECGKSFSTSG |
| | HLVRHQRTHTGEKPYK CPECGKSFSQNSTLTE HQRTHTGKKTSVYVGG |
| | LESRVLKYTAQNMELQ NKVQLLEEQNLSLLDQ LRKLQAMVIEIS |
| 75 | MELELDAGDQDLLAFL LEESGDLGTAPDEAVR APLDWALPLSEVPSDW |
| | EVDDLLCSLLSPPASL NILSSSNPCLVHHDHT YSLPRETVSMDLESES |
| | CRKEGTQMTPQHMEEL AEQEIARLVLTDEEKS LLEKEGLILPETLPLT |
| | KTEEQILKRVRRPYAC PVESCDRRFSRSDNLV RHIRIHTGQKPFQCRI |
| | CMRNFSREDNLHTHIR THTGEKPFACDICGRK FARSDELVRHTKIHLR |
| | QKDRPYACPVESCDRR FSQSGNLTEHIRIHTG QKPFQCRICMRNFSTS |
| | GHLVRHIRTHTGEKPF ACDICGRKFAQNSTLT EHTKIHLRQKDVYVGG |
| | LESRVLKYTAQNMELQ NKVQLLEEQNLSLLDQ LRKLQAMVIEIS |
| 76 | DALDDFDLDMLGSDAL DDFDLDMLGSDALDDF DLDMLGSDALDDFDLD |
| | ML |
| 77 | DALDDFDLDMLGSDAL DDFDLDMLGSDALDDF DLDMLGSDALDDFDLD |
| | MLINSRSSGSPKKKRK VGSQYLPDTDDRHRIE EKRKRTYETFKSIMKK |
| | SPFSGPTDPRPPPRRI AVPSRSSASVPKPAPQ PYPFTSSLSTINYDEF |
| | PTMVFPSGQISQASAL APAPPQVLPQAPAPAP APAMVSALAQAPAPVP |
| | VLAPGPPQAVAPPAPK PTQAGEGTLSEALLQL QFDDEDLGALLGNSTD |
| | PAVFTDLASVDNSEFQ QLLNQGIPVAPHTTEP MLMEYPEAITRLVTGA |
| | QRPPDPAPAPLGAPGL PNGLLSGDEDFSSIAD MDFSALLGSGSGSRDS |
| | REGMFLPKPEAGSAIS DVFEGREVCQPKRIRP FHPPGSPWANRPLPAS |
| | LAPTPTGPVHEPVGSL TPAPVPQPLDPAPAVT PEASHLLEDPDEETSQ |
| | AVKALREMADTVIPQK EEAAICGQMDLSHPPP RGHLDELTTTLESMTE |
| | DLNLDSPLTPELNEIL DTFLNDECLLHAMHIS TGLSIFDTSLF |
| 78 | MSGLEMADHMMAMNHG RFPDGTNGLHHHPAHR MGMGQFPSPHHHQQQQ |
| | PQHAFNALMGEHIHYG AGNMNATSGVRHAMGP GTVNGGHPPSALAPAA |
| | RFNNSQFMGPPVASQG GSLPASMQLQKLNNQY FNHHPYPHNHYMPDLH |
| | PAAGHQMNGTNQHFRD CNPKHSGGSSTPGGSG GSSTPGGSGSSSGGGA |
| | GSSNSGGGSGSGNMPA SVAHVPAAMLPPNVID TDFIDEEVLMSLVIEM |
| | GLDRIKELPELWLGQN EFDFMTDFVCKQQPSR VSC |
| 79 | SGLEMADHMMAMNHGR FPDGTNGLHHHPAHRM GMGQFPSPHHHQQQQP |
| | QHAFNALMGEHIHYGA GNMNATSGVRHAMGPG TVNGGHPPSALAPAAR |
| | FNNSQFMGPPVASQGG SLPASMQLQKLNNQYF NHHPYPHNHYMPDLHP |
| | AAGHQMNGTNQHFRDC NPKHSGGSSTPGGSGG SSTPGGSGSSSGGGAG |
| | SSNSGGGSGSGNMPAS VAHVPAAMLPPNVIDT DFIDEEVLMSLVIEMG |
| | LDRIKELPELWLGQNE FDFMTDFVCKQQPSRV SC |
| 80 | MADHLMLAEGYRLVQR PPSAAAAHGPHALRTL PPYAGPGLDSGLRPRG |
| | APLGPPPPRQPGALAY GAFGPPSSFQPFPAVP PPAAGIAHLQPVATPY |
| | PGRAAAPPNAPGGPPG PQPAPSAAAPPPPAHA LGGMDAELIDEEALTS |
| | LELELGLHRVRELPEL FLGQSEFDCFSDLGSA PPAGSVSC |
| 81 | AADHLMLAEGYRLVQR PPSAAAAHGPHALRTL PPYAGPGLDSGLRPRG |
| | APLGPPPPRQPGALAY GAFGPPSSFQPFPAVP PPAAGIAHLQPVATPY |
| | PGRAAAPPNAPGGPPG PQPAPSAAAPPPPAHA LGGMDAELIDEEALTS |
| | LELELGLHRVRELPEL FLGQSEFDCFSDLGSA PPAGSVSC |
| 82 | MAAAKAEMQLMSPLQI SDPFGSFPHSPTMDNY PKLEEMMLLSNGAPQF |
| | LGAAGAPEGSGSNSSS SSSGGGGGGGGGSNSS SSSSTFNPQADTGEQP |
| | YEHLTAESFPDISLNN EKVLVETSYPSQTTRL PPITYTGRFSLEPAPN |
| | SGNTLWPEPLFSLVSG LVSMTNPPASSSSAPS PAASSASASQSPPLSC |
| | AVPSNDSSPIYSAAPT FPTPNTDIFPEPQSQA FPGSAGTALQYPPPAY |
| | PAAKGGFQVPMIPDYL FPQQQGDLGLGTPDQK PFQGLESRTQQPSLTP |
| | LSTIKAFATQSGSQDL KALNTSYQSQLIKPSR MRKYPNRPSKTPPHER |
| | PYACPVESCDRRFSRS DELTRHIRIHTGQKPF QCRICMRNFSRSDHLT |
| | THIRTHTGEKPFACDI CGRKFARSDERKRHTK IHLRQKDKKADKSVVA |
| | SSATSSLSSYPSPVAT SYPSPVTTSYPSPATT SYPSPVPTSFSSPGSS |
| | TYPSPVHSGFPSPSVA TTYSSVPPAFPAQVSS FPSSAVTNSFSASTGL |
| | SDMTATFSPRTIEIC |
| 83 | MTGKLAEKLPVTMSSL LNQLPDNLYPEEIPSA LNLFSGSSDSVVHYNQ |
| | MATENVMDIGLTNEKP NPELSYSGSFQPAPGN KTVTYLGKFAFDSPSN |
| | WCQDNIISLMSAGILG VPPASGALSTQTSTAS MVQPPQGDVEAMYPAL |
| | PPYSNCGDLYSEPVSF HDPQGNPGLAYSPQDY QSAKPALDSNLFPMIP |
| | DYNLYHHPNDMGSIPE HKPFQGMDPIRVNPPP ITPLETIKAFKDKQIH |
| | PGFGSLPQPPLTLKPI RPRKYPNRPSKTPLHE RPHACPAEGCDRRFSR |
| | SDELTRHLRIHTGHKP FQCRICMRSFSRSDHL TTHIRTHTGEKPFACE |
| | FCGRKFARSDERKRHA KIHLKQKEKKAEKGGA PSASSAPPVSLAPVVT |
| | TCA |
| 84 | Met Ala Pro Lys Lys Lys Arg Lys Val Gly Ile His Gly Val Pro Ala |
| | Ala Leu Glu Pro Gly Glu Lys Pro Tyr Lys Cys Pro Glu Cys Gly Lys |
| | Ser Phe Ser Arg Ser Asp Asn Leu Val Arg His Gln Arg Thr His Thr |
| | Gly Glu Lys Pro Tyr Lys Cys Pro Glu Cys Gly Lys Ser Phe Ser Arg |
| | Glu Asp Asn Leu His Thr His Gln Arg Thr His Thr Gly Glu Lys Pro |
| | Tyr Lys Cys Pro Glu Cys Gly Lys Ser Phe Ser Arg Ser Asp Glu Leu |
| | Val Arg His Gln Arg Thr His Thr Gly Glu Lys Pro Tyr Lys Cys Pro |
| | Glu Cys Gly Lys Ser Phe Ser Gln Ser Gly Asn Leu Thr Glu His Gln |
| | Arg Thr His Thr Gly Glu Lys Pro Tyr Lys Cys Pro Glu Cys Gly Lys |
| | Ser Phe Ser Thr Ser Gly His Leu Val Arg His Gln Arg Thr His Thr |
| | Gly Glu Lys Pro Tyr Lys Cys Pro Glu Cys Gly Lys Ser Phe Ser Gln |
| | Asn Ser Thr Leu Thr Glu His Gln Arg Thr His Thr Gly Lys Lys Thr |
| | Ser Lys Arg Pro Ala Ala Thr Lys Lys Ala Gly Gln Ala Lys Lys Lys |
| | Lys Gly Ser Asp Ala Leu Asp Asp Phe Asp Leu Asp Met Leu Gly Ser |
| | Asp Ala Leu Asp Asp Phe Asp Leu Asp Met Leu Gly Ser Asp Ala Leu |
| | Asp Asp Phe Asp Leu Asp Met Leu Gly Ser Asp Ala Leu Asp Asp Phe |
| | Asp Leu Asp Met Leu |
| 85 | Arg Ser Asp Asn Leu Val Arg |
| 86 | Arg Glu Asp Asn Leu His Thr |
| 87 | Arg Ser Asp Glu Leu Val Arg |
| 88 | Gln Ser Gly Asn Leu Thr Glu |
| 89 | Thr Ser Gly His Leu Val Arg |
| 90 | Gln Asn Ser Thr Leu Thr Glu |
| 91 | ACCGAACCCCGCGTTTATGAACAAACGACCCAACACCGTGCGTTTTATTCTGTCTTTTTA |
| | TTGCCGTCA |
| 92 | Met Glu Lys Arg Leu Gly Val Lys Pro Asn Pro Ala Ser Trp Ile Leu |
| | Ser Gly Tyr Tyr Trp Gln Thr Ser Ala Lys Trp Leu Arg Ser Leu Tyr |
| | Leu Phe Tyr Thr Cys Phe Cys Phe Ser Val Leu Trp Leu Ser Thr Asp |
| | Ala Ser Glu Ser Arg Cys Gln Gln Gly Lys Thr Gln Phe Gly Val Gly |
| | Leu Arg Ser Gly Gly Glu Asn His Leu Trp Leu Leu Glu Gly Thr Pro |
| | Ser Leu Gln Ser Cys Trp Ala Ala Cys Cys Gln Asp Ser Ala Cys His |
| | Val Phe Trp Trp Leu Glu Gly Met Cys Ile Gln Ala Asp Cys Ser Arg |
| | Pro Gln Ser Cys Arg Ala Phe Arg Thr His Ser Ser Asn Ser Met Leu |
| | Val Phe Leu Lys Lys Phe Gln Thr Ala Asp Asp Leu Gly Phe Leu Pro |
| | Glu Asp Asp Val Pro His Leu Leu Gly Leu Gly Trp Asn Trp Ala Ser |
| | Trp Arg Gln Ser Pro Pro Arg Ala Ala Leu Arg Pro Ala Val Ser Ser |
| | Ser Asp Gln Gln Ser Leu Ile Arg Lys Leu Gln Lys Arg Gly Ser Pro |
| | Ser Asp Val Val Thr Pro Ile Val Thr Gln His Ser Lys Val Asn Asp |
| | Ser Asn Glu Leu Gly Gly Leu Thr Thr Ser Gly Ser Ala Glu Val His |
| | Lys Ala Ile Thr Ile Ser Ser Pro Leu Thr Thr Asp Leu Thr Ala Glu |
| | Leu Ser Gly Gly Pro Lys Asn Val Ser Val Gln Pro Glu Ile Ser Glu |
| | Gly Leu Ala Thr Thr Pro Ser Thr Gln Gln Val Lys Ser Ser Glu Lys |
| | Thr Gln Ile Ala Val Pro Gln Pro Val Ala Pro Ser Tyr Ser Tyr Ala |
| | Thr Pro Thr Pro Gln Ala Ser Phe Gln Ser Thr Ser Ala Pro Tyr Pro |
| | Val Ile Lys Glu Leu Val Val Ser Ala Gly Glu Ser Val Gln Ile Thr |
| | Leu Pro Lys Asn Glu Val Gln Leu Asn Ala Tyr Val Leu Gln Glu Pro |
| | Pro Lys Gly Glu Thr Tyr Thr Tyr Asp Trp Gln Leu Ile Thr His Pro |
| | Arg Asp Tyr Ser Gly Glu Met Glu Gly Lys His Ser Gln Ile Leu Lys |
| | Leu Ser Lys Leu Thr Pro Gly Leu Tyr Glu Phe Lys Val Ile Val Glu |
| | Gly Gln Asn Ala His Gly Glu Gly Tyr Val Asn Val Thr Val Lys Pro |
| | Glu Pro Arg Lys Asn Arg Pro Pro Ile Ala Ile Val Ser Pro Gln Phe |
| | Gln Glu Ile Ser Leu Pro Thr Thr Ser Thr Val Ile Asp Gly Ser Gln |
| | Ser Thr Asp Asp Asp Lys Ile Val Gln Tyr His Trp Glu Glu Leu Lys |
| | Gly Pro Leu Arg Glu Glu Lys Ile Ser Glu Asp Thr Ala Ile Leu Lys |
| | Leu Ser Lys Leu Val Pro Gly Asn Tyr Thr Phe Ser Leu Thr Val Val |
| | Asp Ser Asp Gly Ala Thr Asn Ser Thr Thr Ala Asn Leu Thr Val Asn |
| | Lys Ala Val Asp Tyr Pro Pro Val Ala Asn Ala Gly Pro Asn Gln Val |
| | Ile Thr Leu Pro Gln Asn Ser Ile Thr Leu Phe Gly Asn Gln Ser Thr |
| | Asp Asp His Gly Ile Thr Ser Tyr Glu Trp Ser Leu Ser Pro Ser Ser |
| | Lys Gly Lys Val Val Glu Met Gln Gly Val Arg Thr Pro Thr Leu Gln |
| | Leu Ser Ala Met Gln Glu Gly Asp Tyr Thr Tyr Gln Leu Thr Val Thr |
| | Asp Thr Ile Gly Gln Gln Ala Thr Ala Gln Val Thr Val Ile Val Gln |
| | Pro Glu Asn Asn Lys Pro Pro Gln Ala Asp Ala Gly Pro Asp Lys Glu |
| | Leu Thr Leu Pro Val Asp Ser Thr Thr Leu Asp Gly Ser Lys Ser Ser |
| | Asp Asp Gln Lys Ile Ile Ser Tyr Leu Trp Glu Lys Thr Gln Gly Pro |
| | Asp Gly Val Gln Leu Glu Asn Ala Asn Ser Ser Val Ala Thr Val Thr |
| | Gly Leu Gln Val Gly Thr Tyr Val Phe Thr Leu Thr Val Lys Asp Glu |
| | Arg Asn Leu Gln Ser Gln Ser Ser Val Asn Val Ile Val Lys Glu Glu |
| | Ile Asn Lys Pro Pro Ile Ala Lys Ile Thr Gly Asn Val Val Ile Thr |
| | Leu Pro Thr Ser Thr Ala Glu Leu Asp Gly Ser Lys Ser Ser Asp Asp |
| | Lys Gly Ile Val Ser Tyr Leu Trp Thr Arg Asp Glu Gly Ser Pro Ala |
| | Ala Gly Glu Val Leu Asn His Ser Asp His His Pro Ile Leu Phe Leu |
| | Ser Asn Leu Val Glu Gly Thr Tyr Thr Phe His Leu Lys Val Thr Asp |
| | Ala Lys Gly Glu Ser Asp Thr Asp Arg Thr Thr Val Glu Val Lys Pro |
| | Asp Pro Arg Lys Asn Asn Leu Val Glu Ile Ile Leu Asp Ile Asn Val |
| | Ser Gln Leu Thr Glu Arg Leu Lys Gly Met Phe Ile Arg Gln Ile Gly |
| | Val Leu Leu Gly Val Leu Asp Ser Asp Ile Ile Val Gln Lys Ile Gln |
| | Pro Tyr Thr Glu Gln Ser Thr Lys Met Val Phe Phe Val Gln Asn Glu |
| | Pro Pro His Gln Ile Phe Lys Gly His Glu Val Ala Ala Met Leu Lys |
| | Ser Glu Leu Arg Lys Gln Lys Ala Asp Phe Leu Ile Phe Arg Ala Leu |
| | Glu Val Asn Thr Val Thr Cys Gln Leu Asn Cys Ser Asp His Gly His |
| | Cys Asp Ser Phe Thr Lys Arg Cys Ile Cys Asp Pro Phe Trp Met Glu |
| | Asn Phe Ile Lys Val Gln Leu Arg Asp Gly Asp Ser Asn Cys Glu Trp |
| | Ser Val Leu Tyr Val Ile Ile Ala Thr Phe Val Ile Val Val Ala Leu |
| | Gly Ile Leu Ser Trp Thr Val Ile Cys Cys Cys Lys Arg Gln Lys Gly |
| | Lys Pro Lys Arg Lys Ser Lys Tyr Lys Ile Leu Asp Ala Thr Asp Gln |
| | Glu Ser Leu Glu Leu Lys Pro Thr Ser Arg Ala Gly Ile Lys Gln Lys |
| | Gly Leu Leu Leu Ser Ser Ser Leu Met His Ser Glu Ser Glu Leu Asp |
| | Ser Asp Asp Ala Ile Phe Thr Trp Pro Asp Arg Glu Lys Gly Lys Leu |
| | Leu His Gly Gln Asn Gly Ser Val Pro Asn Gly Gln Thr Pro Leu Lys |
| | Ala Arg Ser Pro Arg Glu Glu Ile Leu |
| 93 | Leu Glu Pro Gly Glu Lys Pro Tyr Lys Cys Pro Glu Cys Gly Lys Ser |
| | Phe Ser Arg Ser Asp Asn Leu Val Arg His Gln Arg Thr His Thr Gly |
| | Glu Lys Pro Tyr Lys Cys Pro Glu Cys Gly Lys Ser Phe Ser Arg Glu |
| | Asp Asn Leu His Thr His Gln Arg Thr His Thr Gly Glu Lys Pro Tyr |
| | Lys Cys Pro Glu Cys Gly Lys Ser Phe Ser Arg Ser Asp Glu Leu Val |
| | Arg His Gln Arg Thr His Thr Gly Glu Lys Pro Tyr Lys Cys Pro Glu |
| | Cys Gly Lys Ser Phe Ser Gln Ser Gly Asn Leu Thr Glu His Gln Arg |
| | Thr His Thr Gly Glu Lys Pro Tyr Lys Cys Pro Glu Cys Gly Lys Ser |
| | Phe Ser Thr Ser Gly His Leu Val Arg His Gln Arg Thr His Thr Gly |
| | Glu Lys Pro Tyr Lys Cys Pro Glu Cys Gly Lys Ser Phe Ser Gln Asn |
| | Ser Thr Leu Thr Glu His Gln Arg Thr His Thr Gly Lys Lys Thr Ser |
| 94 | CCTGCAGGCAGCTGCGCGCTCGCTCGCTCACTGAGGCCGCCCGGGCAAAGCCCGGG |
| | CGTCGGGCGACCTTTGGTCGCCCGGCCTCAGTGAGCGAGCGAGCGCGCAGAGAGGG |
| | AGTGGCCAACTCCATCACTAGGGGTTCCTGCGGCCGCACGCGTGGCGCGCCggaggaag |
| | ccatcaactaaactacaatgactgtaagatacaaaattgggaatggtaacatattttgaagttctgttgacataaagaatcatgatat |
| | taatgcccatggaaatgaaagggcgatcaacactatggtttgaaaagggggaaattgtagagcacagatgtgttcgtgtggcagt |
| | gtgctgtctctagcaatactcagagaagagagagaacaatgaaattctgattggccccagtgtgagcccagatgaggttcagctgccaact |
| | ttctctttcacatcttatgaaagtcatttaagcacaactaactttttttttttttttttttttttgagacagagtcttgctctgttgcccagg |
| | acagagtgcagtagtgactcaatctcggctcactgcagcctccacctcctaggctcaaacggtcctcctgcatcagcctcccaagt |
| | agctggaattacaggagtggcccaccatgcccagctaatttttgtatttttaatagatacgggggtttcaccatatcacccaggctggt |
| | ctcgaactcctggcctcaagtgatccacctgcctcggcctcccaaagtgctgggattataggcgtcagccactatgcccaacccg |
| | accaaccttttttaaaataaatatttaaaaaattggtatttcacatatatactagtatttacatttatccacacaaaacggacgggcctc |
| | cgctgaaccagtgaggccccagacgtgcgcataaataacccctgcgtgctgcaccacctggggagagggggaggaccacgg |
| | taaatggagcgagcgcatagcaaaagggacgcggggtccttttctctgccggtggcactgggtagctgtggccaggtgtggtact |
| | ttgatggggcccagggctggagctcaaggaagcgtcgcagggtcacagatctgggggaaccccggggaaaagcactgaggc |
| | aaaaccgccgctcgtctcctacaatatatgggagggggaggttgagtacgttctggattactcataagaccttttttttttccttccggg |
| | cgcaaaaccgtgagctggatttataatcgccctataaagctccagaggcggtcaggcacctgcagaggagccccgccgctccg |
| | ccgactagctgcccccgcgagcaacggcctcgtgatttccccgccgatccggtccccgcctccccactctgcccccgcctacccc |
| | ggagccgtgcagccgcctctccgaatctctctcttctcctggcgctcgcgtgcgagagggaactagcgagaacgaggaagcag |
| | ctggaggtgacgccgggcagattacgcctgtcagggccgagccgagcggatcgctgggcgctgtgcagaggaaaggcggga |
| | gtgcccggctcgctgtcgcagagccgaggtgggtaagctagcgaccacctggacttcccagcgcccaaccgtggcttttcagcc |
| | aggtcctctcctcccgcggcttctcaaccaaccccatcccagcgccggccacccaacctcccgaaatgagtgcttcctgccccag |
| | cagccgaaggcgctactaggaacggtaacctgttacttttccaggggccgtagtcgacccgctgcccgagttgctgtgcgactgc |
| | gcgcgcggggctagagtgcaaggtgactgtggttcttctctggccaagtccgagggagaacgtaaagatatgggcctttttccccc |
| | tctcaccttgtctcaccaaagtccctagtccccggagcagttagcctctttctttccagggaattagccagacacaacaacgggaac |
| | cagacaccgaaccagacatgcccgccccgtgcgccctccccgctcgctgcctttcctccctcttgtctctccagagccggatcttca |
| | aggggagcctccgtgcccccggctgctcagtccctccggtgtgcaggaccccggaagtcctccccgcacagctctcgcttctcttt |
| | gcagcctgtttctgcgccggaccagtcgaggactctggacagtagaggccccgggacgaccgagctgGAATTCGCCAC |
| | CATGGCCCCAAAGAAGAAGCGGAAGGTCGGTATCCACGGAGTCCCAGCAGCCCTCGA |
| | ACCAGGTGAAAAACCTTACAAATGTCCTGAATGTGGGAAATCATTCAGTCGCAGCGACA |
| | ACCTGGTGAGACATCAACGCACCCATACAGGAGAAAAACCTTATAAATGTCCAGAATGT |
| | GGAAAGTCCTTCTCACGAGAGGATAACTTGCACACTCATCAACGAACACATACTGGTGA |
| | AAAACCATACAAGTGTCCCGAATGTGGTAAAAGTTTTAGCCGGAGCGATGAACTTGTCC |
| | GACACCAACGAACCCATACAGGCGAGAAGCCTTACAAATGTCCCGAGTGTGGCAAGAG |
| | CTTCTCACAATCAGGGAATCTGACTGAGCATCAACGAACTCATACCGGGGAAAAACCTT |
| | ACAAGTGTCCAGAGTGTGGGAAGAGCTTTTCCACAAGTGGACATCTGGTACGCCACCA |
| | GAGGACACATACAGGGGAGAAGCCCTACAAATGCCCCGAATGCGGTAAAAGTTTCTCT |
| | CAGAATAGTACCCTGACCGAACACCAGCGAACACACACTGGGAAAAAAACGAGTAAAA |
| | GGCCGGCGGCCACGAAAAAGGCCGGCCAGGCAAAAAAGAAAAAGGGATCCGACGCGC |
| | TGGACGATTTCGATCTCGACATGCTGGGTTCTGATGCCCTCGATGACTTTGACCTGGAT |
| | ATGTTGGGAAGCGACGCATTGGATGACTTTGATCTGGACATGCTCGGCTCCGATGCTCT |
| | GGACGATTTCGATCTCGATATGTTATAAAAAGAGACCGGTTCACTGTGACAGTAAAAGA |
| | GACCGGTTCACTGTGAGAATGAAAGAGACCGGTTCACTGTGATCGGAAAAGAGACCGG |
| | TTCACTGTGAGCGGCCTTGAAACCCAGCAGACAATGTAGCTCAGTAGAAACCCAGCAG |
| | ACAATGTAGCTGAATGGAAACCCAGCAGACAATGTAGCTTCGGAGAAACCCAGCAGAC |
| | AATGTAGCTAAGCTTGGGTGGCATCCCTGTGACCCCTCCCCAGTGCCTCTCCTGGCCC |
| | TGGAAGTTGCCACTCCAGTGCCCACCAGCCTTGTCCTAATAAAATTAAGTTGCATCATTT |
| | TGTCTGACTAGGTGTCCTTCTATAATATTATGGGGTGGAGGGGGGTGGTATGGAGCAA |
| | GGGGCAAGTTGGGAAGACAACCTGTAGGGCCTGCGGGGTCTATTGGGAACCAAGCTG |
| | GAGTGCAGTGGCACAATCTTGGCTCACTGCAATCTCCGCCTCCTGGGTTCAAGCGATTC |
| | TCCTGCCTCAGCCTCCCGAGTTGTTGGGATTCCAGGCATGCATGACCAGGCTCAGCTA |
| | ATTTTTGTTTTTTTGGTAGAGACGGGGTTTCACCATATTGGCCAGGCTGGTCTCCAACTC |
| | CTAATCTCAGGTGATCTACCCACCTTGGCCTCCCAAATTGCTGGGATTACAGGCGTGAA |
| | CCACTGCTCCCTTCCCTGTCCTTCACGTGCGGACCGAGCGGCCGCAGGAACCCCTAGT |
| | GATGGAGTTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAGCGAGCGC |
| | GCAGCTGCCTGCAGG |
| 95 | LEPGEKPYKCPECGKSFSRSDNLVRHQRTHTGEKPYKCPECGKSFSHRTTLTNHQRTHTG |
| | EKPYKCPECGKSFSREDNLHTHQRTHTGEKPYKCPECGKSFSTSHSLTEHQRTHTGEKPY |
| | KCPECGKSFSQSSSLVRHQRTHTGEKPYKCPECGKSFSREDNLHTHQRTHTGKKTS |
| 96 | CCTGCAGGCAGCTGCGCGCTCGCTCGCTCACTGAGGCCGCCCGGGCGTCGGGCGAC |
| | CTTTGGTCGCCCGGCCTCAGTGAGCGAGCGAGCGCGCAGAGAGGGAGTGGCCAACTC |
| | CATCACTAGGGGTTCCTGCGGCCGCACGCGTGGCGCGCCGTTTAAACTTAATTAAGCT |
| | AGCCGTTACATAACTTACGGTAAATGGCCCGCCTGGCTGACCGCCCAACGACCCCCGC |
| | CCATTGACGTCAATAATGACGTATGTTCCCATAGTAACGCCAATAGGGACTTTCCATTGA |
| | CGTCAATGGGTGGAGTATTTACGGTAAACTGCCCACTTGGCAGTACATCAAGTGTATCA |
| | TATGCCAAGTACGCCCCCTATTGACGTCAATGACGGTAAATGGCCCGCCTGGCATTATG |
| | CCCAGTACATGACCTTATGGGACTTTCCTACTTGGCAGTACATCTACGTATTAGTCATCG |
| | CTATTACCATGggtcgaggtgagccccacgttctgcttcactctccccatctcccccccctccccacccccaattttgtatttatt |
| | tattttttaattattttgtgcagcgatgggggcggggggggggggggcgcgcgccaggcggggcggggcggggcgaggggcg |
| | gggcggggcgaggcggagaggtgcggcggcagccaatcagagcggcgcgctccgaaagtttccttttatggcgaggcggcg |
| | gcggcggcggccctataaaaagcgaagcgcgcggcgggcgggagtcgctgcgttgccttcgccccgtgccccgctccgcgcc |
| | gcctcgcgccgcccgccccggctctgactgaccgcgttactcccacaggtgagcgggcgggacggcccttctcctccgggctgt |
| | aattagcgcttggtttaatgacggctcgtttcttttctgtggctgcgtgaaagccttaaagggctccgggagggccctttgtgcggggg |
| | ggagcggctcggggggtgcgtgcgtgtgtgtgtgcgtggggagcgccgcgtgcggcccgcgctgcccggcggctgtgagcgct |
| | gcgggcgcggcgcggggctttgtgcgctccgcgtgtgcgcgaggggagcgcggccgggggcggtgccccgcggtgcggggg |
| | ggctgcgaggggaacaaaggctgcgtgcggggtgtgtgcgtgggggggtgagcagggggtgtgggcgcggcggtcgggctg |
| | taacccccccctgcacccccctccccgagttgctgagcacggcccggcttcgggtgcggggctccgtgcggggcgtggcgcgg |
| | ggctcgccgtgccgggcggggggtggcggcaggtgggggtgccgggcggggcggggccgcctcgggccggggagggctc |
| | gggggaggggcgcggcggccccggagcgccggcggctgtcgaggcgcggcgagccgcagccattgccttttatggtaatcgt |
| | gcgagagggcgcagggacttcctttgtcccaaatctggcggagccgaaatctgggaggcgccgccgcaccccctctagcggg |
| | cgcgggcgaagcggtgcggcgccggcaggaaggaaatgggcggggagggccttcgtgcgtcgccgcgccgccgtccccttc |
| | tccatctccagcctcggggctgccgcagggggacggctgccttcgggggggacggggcagggcggggttcggcttctggcgtgt |
| | gaccggcggctctagagcctctgctaaccatgttcatgccttcttctttttcctacagctcctgggcaacgtgctggttgttgtgctgtctc |
| | atcattttggcaaagaattGAATTCGCCACCATGGCCCCAAAGAAGAAGCGGAAGGTCGGTATCC |
| | ACGGAGTCCCAGCAGCCCTTGAGCCCGGTGAGAAGCCTTATAAATGCCCTGAATGCGG |
| | GAAAagcTTCAGCCGCTCAGACAACCTCGTTCGACACCAGCGgACACATACCGGTGAAA |
| | AGCCATACAAATGCCCAGAATGTGGGAAGTCATTCTCCCACCGGACTACACTCACGAAC |
| | CATCAgcgcACTCATACGGGAGAGAAACCCTATAAGTGCCCGGAATGCGGGAAATCATTC |
| | AGTAGAGAAGACAATCTCCATACTCATCAACGAACTCACACAGGCGAGAAGCCATACAA |
| | GTGCCCAGAATGCGGGAAATCATTTTCTACCAGCCATTCTCTCACTGAACACCAGAGAA |
| | CCCACACGGGGGAAAAGCCGTACAAGTGTCCAGAGTGTGGAAAGTCCTTCAGCCAGTC |
| | TAGCTCACTGGTGAGGCATCAGAGGACTCATACCGGCGAAAAACCCTATAAGTGCCCC |
| | GAGTGTGGGAAAAGTTTTTCCAGGGAGGATAACCTGCATACGCATCAAAGGACCCATAC |
| | CGGTAAAAAAACATCCAAAAGGCCGGCGGCCACGAAAAAGGCCGGCCAGGCAAAAAAG |
| | AAAAAGGGATCCTACCCATACGACGTACCAGATTACGCTCTCGAGGACGCGCTGGACG |
| | ATTTCGATCTCGACATGCTGGGTTCTGATGCCCTCGATGACTTTGACCTGGATATGTTG |
| | GGAAGCGACGCATTGGATGACTTTGATCTGGACATGCTCGGCTCCGATGCTCTGGACG |
| | ATTTCGATCTCGATATGTTATAAACTAGTaataaaagatctttattttcattagatctgtgtgttggttttttgtgtgCA |
| | CGTGCGGACCGAGCGGCCGCAGGAACCCCTAGTGATGGAGTTGGCCACTCCCTCTCT |
| | GCGCGCTCGCTCGCTCACTGAGGCCGGGCGACCAAAGGTCGCCCGACGCCCGGGCTT |
| | TGCCCGGGCGGCCTCAGTGAGCGAGCGAGCGCGCAGCTGCCTGCAGG |

What is claimed is:

1. A nucleic acid comprising a response element operably linked to a reporter nucleotide sequence, wherein the response element comprises from 2 to 10 copies of a Z1 transcriptional factor (TF) binding site.

2. The nucleic acid of claim 1, wherein the response element comprises from 3 to 8 copies of the Z1 TF binding site.

3. The nucleic acid of claim 1 or claim 2, wherein each of the copies of the Z1 TF binding site has a nucleic acid sequence of SEQ ID NO: 1 or a nucleic acid sequence having 1, 2, or 3 nucleotide differences from SEQ ID NO:1.

4. The nucleic acid of any one of claims 1-3, wherein the response element comprises a spacer sequence between at least two copies of the Z1 TF binding site.

5. The nucleic acid of claim 4, wherein the spacer sequence comprises the sequence of SEQ ID NO: 2 or SEQ ID NO: 3.

6. The nucleic acid of any one of claims 1-4, comprising a sequence of any one of SEQ ID NOs: 10-15, SEQ ID NOs: 17-23, and SEQ ID NO: 25

7. The nucleic acid of claim 1 or claim 2, wherein the response element further comprises a promoter.

8. The nucleic acid of claim 7, wherein the promoter is a minimal promoter.

9. The nucleic acid of claim 8, wherein the minimal promoter is any one of a CMV-minimal promoter, a hsp70 minimal promoter, a minimal promoter included in a tetracycline response element, or a MinTk minimal promoter.

10. The nucleic acid of claim 9, wherein the minimal promoter comprises the sequence of SEQ ID NO: 32.

11. The nucleic acid of any one of claims 1-10, wherein the reporter nucleotide sequence encodes a luminescent protein or an enzyme that produces bioluminescence.

12. The nucleic acid of claim 11, wherein the luminescent protein is green fluorescent protein (GFP), enhanced GFP (EGFP), or mCherry.

13. The nucleic acid of claim 11, wherein the enzyme is a luciferase.

14. The nucleic acid of any one of claims 1-13, further comprising a polyA signal sequence operably linked to the reporter nucleic acid sequence.

15. The nucleic acid of claim 14, wherein the polyA signal sequence comprises SEQ ID NO: 91.

16. The nucleic acid of any one of claims 1-15, comprising a second reporter nucleotide sequence operably linked to a constitutive promoter.

17. A cell comprising the nucleic acid of any one of claims 1-16.

18. The cell of claim 17, wherein the nucleic acid is stably integrated into the genome of the cell.

19. The cell of claim 17 or claim 18, wherein the cell is engineered to stably overexpress an adeno-associated virus receptor (AAVR).

20. The cell of claim 19, wherein the AAVR is wild-type AAVR.

21. The cell of any one of claims 17-20, further comprising a transcription factor (TF) that binds to the Z1 TF binding site.

22. The cell of claim 21, wherein the TF is expressed from an exogenous nucleic acid.

23. The cell of claim 22, wherein the exogenous nucleic acid is present in an AAV vector.

24. The cell of claim 23, wherein the AAV vector is an AAV9 vector or an scAAV9 vector.

25. The cell of any one of claims 21-24, wherein the TF is a transcriptional activator.

26. The cell of any one of claims 21-25, wherein the TF comprises an engineered Z1 binding domain.

27. The cell of claim 26, wherein the Z1 binding domain comprises SEQ ID NO: 93.

28. A cell comprising a nucleic acid comprising a response element operably linked to a reporter nucleotide sequence, wherein the cell is further engineered to stably overexpress an adeno-associated virus receptor (AAVR).

29. The cell of claim 28, wherein the response element comprises from 2 to 10 copies of a transcription factor (TF) binding site.

30. The cell of claim 29, wherein the response element comprises from 3 to 8 copies of the TF binding site.

31. The cell of claim 29, wherein the response element comprises from 3 to 6 copies of the TF binding site.

32. The cell of any one of claims 29-31, wherein the TF binding site comprises a sequence bound by an endogenous TF.

33. The cell of claim 32, wherein the endogenous TF is ligand-dependent TF.

34. The cell of claim 33, wherein the ligand is a metal.

35. The cell of claim 34, wherein the metal is copper.

36. The cell of claim 35, wherein the TF binding site comprises SEQ ID NO: 8.

37. The cell of claim 36, wherein the endogenous TF is metal-responsive transcription factor 1 (MTF-1).

38. The cell of any one of claims 29-31, wherein the TF binding site comprises a sequence bound by an exogenous TF.

39. The cell of claim 38, wherein the exogenous TF comprises an engineered DNA binding domain specific for the TF binding site.

40. The cell of claim 39, wherein the engineered DNA binding domain comprises from 2 to 10 zinc fingers.

41. The cell of claim 40, wherein the TF binding site comprises the sequence of SEQ ID NO: 1.

42. The cell of claim 41, wherein the engineered DNA binding domain is selected from the group consisting of SEQ ID NOs: 76-83.

43. The cell of any one of claims 38-42, wherein the exogenous TF is expressed in the cell from an expression vector.

44. The cell of claim 43, wherein the expression vector is an AAV.

45. The cell of any one of claims 28-44, wherein the response element further comprises a promoter.

46. The cell of claim 45, wherein promoter is a minimal promoter.

47. The cell of any one of claims 28-46, wherein the reporter nucleotide sequence encodes a luminescent protein or an enzyme that produces bioluminescence.

48. The cell of claim 47, wherein the luminescent protein is green fluorescent protein (GFP), enhanced GFR (EGFR), or mCherry.

49. The cell of any one of claims 28-48, wherein the nucleic acid further comprises a polyA signal sequence operably linked to the reporter nucleic acid sequence.

50. The cell of claim 19, wherein the polyA signal sequence comprises SEQ ID NO: 91.

51. The cell of any one of claims 28-50, wherein the AAVR is wild type AAVR.

52. A method of determining the potency of a sample comprising an AAV vector, comprising: (a) contacting the cell of any one of claims 28-51 with all or part of the sample, wherein the expression vector encodes a modulator that directly or indirectly modulates expression of the reporter nucleotide sequence via the response element, (b) measuring expression of the reporter nucleotide sequence in the cell, and (c) determining the potency of the sample based on the measured expression level of the reporter nucleic acid sequence.

53. The method of claim 52, wherein the modulator is a transcription factor that binds to TF binding sites in the response element.

54. The method of claim 52, wherein the modulator is a protein that regulates metal metabolism, wherein the response element is a metal-responsive response element.

55. The method of any one of claims 52-54, wherein the AAV vector is an AAV9 vector or an scAAV9 vector.

56. The method of any one of claims 52-55, wherein the determining step (c) comprises comparing the measured expression level to a standard potency curve for the expression vector delivery vehicle.

57. The nucleic acid of claim 16, wherein the second reporter nucleotide sequence encodes a luminescent protein or an enzyme that produces bioluminescence.

58. The nucleic acid of claim 57, wherein the luminescent protein is green fluorescent protein (GFP), enhanced GFP (EGFP), or mCherry.

59. The nucleic acid of claim 57, wherein the enzyme is a luciferase.

60. The nucleic acid of any one of claims 16 and 57-59, wherein the constitutive promoter is a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Ad E1A promoter, or a cytomegalovirus (CMV) promoter.

61. A nucleic acid comprising a response element operably linked to a reporter nucleotide sequence, wherein the response element comprises a transcriptional factor (TF) binding site bound by an endogenous metal-dependent TF.

62. The nucleic acid of claim 61, wherein the metal is copper.

63. The nucleic acid of claim 61 or claim 62, wherein the metal-dependent TF is metal response element-binding transcription factor-1 (MTF-1).

64. The nucleic acid of claim 61 or 62, wherein the TF binding site comprises SEQ ID NO: 8.

65. The nucleic acid of any one of claims 61-65, wherein the response element further comprises a promoter.

66. The nucleic acid of claim 65, wherein the promoter is a minimal promoter.

67. The nucleic acid of claim 66, wherein the minimal promoter is any one of a CMV-minimal promoter, a hsp70 minimal promoter, a minimal promoter included in a tetracycline response element, or a MinTk minimal promoter.

68. The nucleic acid of claim 66, wherein the minimal promoter comprises the sequence of SEQ ID NO: 32.

69. The nucleic acid of any one of claims 61-68, wherein the reporter nucleotide sequence encodes a luminescent protein or an enzyme that produces bioluminescence.

70. The nucleic acid of claim 69, wherein the luminescent protein is green fluorescent protein (GFP), enhanced GFP (EGFP), or mCherry.

71. The nucleic acid of claim 69, wherein the enzyme is a luciferase.

72. The nucleic acid of any one of claims 61-71, further comprising a polyA signal sequence operably linked to the reporter nucleic acid sequence.

73. The nucleic acid of claim 72, wherein the polyA signal sequence comprises SEQ ID NO: 91.

74. The nucleic acid of any one of claims 61-73, comprising a second reporter nucleotide sequence operably linked to a constitutive promoter.

75. The nucleic acid of claim 74, wherein the second reporter nucleotide sequence encodes a luminescent protein or an enzyme that produces bioluminescence.

76. The nucleic acid of claim 75, wherein the luminescent protein is green fluorescent protein (GFP), enhanced GFP (EGFP), or mCherry.

77. The nucleic acid of claim 75, wherein the enzyme is a luciferase.

78. The nucleic acid of any one of claims 74-77, wherein the constitutive promoter is a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, aa Ad E1A promoter, or a cytomegalovirus (CMV) promoter.

79. A cell comprising the nucleic acid of any one of claims 61-78.

80. The cell of claim 79, wherein the nucleic acid is stably integrated into the genome of the cell.

81. The cell of claim 79 or claim 80, wherein the cell is engineered to stably overexpress an adeno-associated virus receptor (AAVR).

82. The cell of claim 81, wherein the AAVR is wild-type AAVR.

83. The cell of any one of claims 79-82, further comprising a transcription factor (TF) that binds to the TF binding site.

84. The cell of claim 83, wherein the TF is expressed from an exogenous nucleic acid.

85. The cell of claim 84, wherein the exogenous nucleic acid is present in an AAV vector.

86. The cell of claim 85, wherein the AAV vector is an AAV9 vector or an scAAV9 vector.

87. The cell of any one of claims 83-86, wherein the TF is MTF-1.


SEQUENCE LISTING (96 sequences)

SEQ ID NO: 1 - DNA, 18 bp, Synthetic construct
  source 1..18: mol_type = other DNA
ctaggtcaagtgtaggag

SEQ ID NO: 2 - skipped

SEQ ID NO: 3 - DNA, 18 bp, Synthetic construct
  source 1..18: mol_type = other DNA
gatagggagtaaactcga

SEQ ID NO: 4-8 - skipped (5 entries)

SEQ ID NO: 9 - DNA, 315 bp, Synthetic construct
  source 1..315: mol_type = other DNA
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 10 - DNA, 316 bp, Synthetic construct
  source 1..316: mol_type = other DNA
gagtttactccctatcctaggtcaagtgtaggagtcgagtttactccctatcctaggtcaagtgtaggagtcgagtttactccctatcctaggtcaagtgtaggagtcgagtttactccctatcctaggtcaagtgtaggagtcgagtttactccctatcctaggtcaagtgtaggagtcgagtttatccctatcctaggtcaagtgtaggagtcgagtttactccctatcctaggtcaagtgtaggagtcgaggtaggcgtgtacggtgggaggcctatataagcagagctcgtttagtgaaccgtcagatcgcc

SEQ ID NO: 11 - DNA, 236 bp, Synthetic construct
  source 1..236: mol_type = other DNA
tccctatcctaggtcaagtgtaggagtcgagtttactccctatcctaggtcaagtgtaggagtcgagtttactccctatcctaggtcaagtgtaggagtcgagtttatccctatcctaggtcaagtgtaggagtcgagtttactccctatcctaggtcaagtgtaggagtcgaggtaggcgtgtacggtgggaggcctatataagcagagctcgtttagtgaaccgtcagatcgcc

SEQ ID NO: 12 - DNA, 164 bp, Synthetic construct
  source 1..164: mol_type = other DNA
tccctatcctaggtcaagtgtaggagtcgagtttatccctatcctaggtcaagtgtaggagtcgagtttactccctatcctaggtcaagtgtaggagtcgaggtaggcgtgtacggtgggaggcctatataagcagagctcgtttagtgaaccgtcagatcgcc

SEQ ID NO: 13 - DNA, 93 bp, Synthetic construct
  source 1..93: mol_type = other DNA
tccctatcctaggtcaagtgtaggagtcgaggtaggcgtgtacggtgggaggcctatataagcagagctcgtttagtgaaccgtcagatcgcc

SEQ ID NO: 14 - DNA, 176 bp, Synthetic construct
  source 1..176: mol_type = other DNA
ctaggtcaagtgtaggagatttgcctaggtcaagtgtaggagctcgatctaggtcaagtgtaggagctcgatctaggtcaagtgtaggagatttgcctaggtcaagtgtaggagctcgatctaggtcaagtgtaggaggctagctagagggtatataatggaagctcgacttccag

SEQ ID NO: 15 - DNA, 104 bp, Synthetic construct
  source 1..104: mol_type = other DNA
ctaggtcaagtgtaggagctcgatctaggtcaagtgtaggagctcgatctaggtcaagtgtaggaggctagctagagggtatataatggaagctcgacttccag

SEQ ID NO: 16 - DNA, 176 bp, Synthetic construct
  source 1..176: mol_type = other DNA
agtgatagagaacgtatgatttgcagtgatagagaacgtatgctcgatagtgatagagaacgtatgctcgatagtgatagagaacgtatgatttgcagtgatagagaacgtatgctcgatagtgatagagaacgtatggctagctagagggtatataatggaagctcgacttccag

SEQ ID NO: 17 - DNA, 544 bp, Synthetic construct
  source 1..544: mol_type = other DNA
ctaggtcaagtgtaggagatttgcctaggtcaagtgtaggagctcgatctaggtcaagtgtaggagctcgatctaggtcaagtgtaggagatttgcctaggtcaagtgtaggagctcgatctaggtcaagtgtaggaggctagcaaaaggctgagagaaaagaggttgaggaagaaatcataaatctggattgtgagaaagtgtttaatatttagccactagatggcgatgtaatgtaaggtgctgtcttgactttttttttttttttttgaaacaagctatttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttt

SEQ ID NO: 18 - DNA, 544 bp, Synthetic construct
  source 1..544: mol_type = other DNA
ctaggtcaagtgtaggagatttgcctaggtcaagtgtaggagctcgatctaggtcaagtgtaggagctcgatctaggtcaagtgtaggagatttgcctaggtcaagtgtaggagctcgatctaggtcaagtgtaggaggctagcgcctttatacgcacagtctccatcttctgggtgtgtcacagactgaataactcattagtgatatagtgttaattagcctaatgtactttgattaaggccatatttgaaccacttttaaaactcatggcacagttcctgtatcagtaaagcacaagaattaataaataagtgatgcttaactaaactcaacacagaaaccatttgtgttaaaattttttctttagaaatcacctttcaatttaaggagaaaacagactttaaatcctctagctcatgtttcatgacaagaatttatttatattaacatctcttagtccactctttaaaatatctgtattccttttattttaggaatttcatatgcagaataaatggtaattaaaatgtgcaggatgacaag

SEQ ID NO: 19 - DNA, 472 bp, Synthetic construct
  source 1..472: mol_type = other DNA
ctaggtcaagtgtaggagctcgatctaggtcaagtgtaggagctcgatctaggtcaagtgtaggaggctagcaaaaggctgagagaaaagaggttgaggaagaaatcataaatctggattgtgagaaagtgtttaatatttagccactagatggcgatgtaatgtaaggtgctgtcttgactttttttttttttttttgaaacaagctatttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttt

SEQ ID NO: 20 - DNA, 245 bp, Synthetic construct
  source 1..245: mol_type = other DNA
ttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggg

SEQ ID NO: 21 - DNA, 684 bp, Synthetic construct
  source 1..684: mol_type = other DNA
caaaatatttcaaaagaggaagacaagaaagacttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggggctagcaaaaggctgagagaaaagaggttgaggaagaaatcataaatctggattgtgagaaagtgtttaatatttagccactagatggcgatgtaatgtaaggtgctgtcttgactttttttttttttttttgaaacaagctatttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttt

SEQ ID NO: 22 - DNA, 690 bp, Synthetic construct
  source 1..690: mol_type = other DNA
caaaatatttcaaaagaggaagacaagaaagacttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggggctagcggtaccgcctttatacgcacagtctccatcttctgggtgtgtcacagactgaataactcattagtgatatagtgttaattagcctaatgtactttgattaaggccatatttgaaccacttttaaaactcatggcacagttcctgtatcagtaaagcacaagaattaataaataagtgatgcttaactaaactcaacacagaaaccatttgtgttaaaattttttctttagaaatcacctttcaatttaaggagaaaacagactttaaatcctctagctcatgtttcatgacaagaatttatttatattaacatctcttagtccactctttaaaatatctgtattccttttattttaggaatttcatatgcagaataaatggtaattaaaatgtgcaggatgacaag

SEQ ID NO: 23 - DNA, 1090 bp, Synthetic construct
  source 1..1090: mol_type = other DNA
caaaatatttcaaaagaggaagacaagaaagacttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggggctagcaaaaggctgagagaaaagaggttgaggaagaaatcataaatctggattgtgagaaagtgtttaatatttagccactagatggcgatgtaatgtaaggtgctgtcttgactttttttttttttttttgaaacaagctatttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttcctttggtaccgcctttatacgcacagtctccatcttctgggtgtgtcacagactgaataactcattagtgatatagtgttaattagcctaatgtactttgattaaggccatatttgaaccacttttaaaactcatggcacagttcctgtatcagtaaagcacaagaattaataaataagtgatgcttaactaaactcaacacagaaaccatttgtgttaaaattttttctttagaaatcacctttcaatttaaggagaaaacagactttaaatcctctagctcatgtttcatgacaagaatttatttatattaacatctcttagtccactctttaaaatatctgtattccttttattttaggaatttcatatgcagaataaatggtaattaaaatgtgcaggatgacaag

SEQ ID NO: 24 - DNA, 245 bp, Synthetic construct
  source 1..245: mol_type = other DNA
ttcatgatttccatgcaaactcccagcctgcctggctgcctgctgctgccaatacttcttgctcttgctggtgatgacattcagagagctccccagcactggtgcttcgttatgtaaggctgtagtgatagagaacgtatgacacactgctggcctgtggaaactcatggaactgttcctccagattaacacttcaggggttatggaagctggaggaagctgagcttttactacatcttttgggg

SEQ ID NO: 25 - DNA, 283 bp, Synthetic construct
  source 1..283: mol_type = other DNA
ttcatgatttccatgcaaactcccagcctgcctggctgcctgctgctgccaatacttcttgctcttgctggtgatgacattcagagagctccccagcactggtgcttcgttatgtaaggctgtctaggtcaagtgtaggagacacactgctggcctgtggaaactcatggaactgttcctccagattaacacttcaggggttatggaagctggaggaagctgagcttttactacatcttttgggggctagctagagggtatataatggaagctcgacttccag

SEQ ID NO: 26 - DNA, 283 bp, Synthetic construct
  source 1..283: mol_type = other DNA
ttcatgatttccatgcaaactcccagcctgcctggctgcctgctgctgccaatacttcttgctcttgctggtgatgacattcagagagctccccagcactggtgcttcgttatgtaaggctgtagtgatagagaacgtatgacacactgctggcctgtggaaactcatggaactgttcctccagattaacacttcaggggttatggaagctggaggaagctgagcttttactacatcttttgggggctagctagagggtatataatggaagctcgacttccag

SEQ ID NO: 27 - DNA, 477 bp, Synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 28 - DNA, 62 bp, Synthetic construct
  source 1..62: mol_type = other DNA
aataaaagatctttattttcattagatctgtgtgttggttttttgtgtgcggaccgcacgtg

SEQ ID NO: 29 - protein, 52 aa, synthetic construct
  SITE 19: note = Recognition Helix
  SITE 41: note = Recognition Helix
LEPGEKPYKCPECGKSFSXHQRTHTGEKPYKCPECGKSFSXHQRTHTGKKTS

SEQ ID NO: 30 - DNA, 4398 bp, Synthetic construct
  source 1..4398: mol_type = other DNA
atgcccgaacaggagcgccagattactgccagagagggagcatccagaaaaatcctgagcaaactgtcactgcccacacgagcttgggaacccgcaatgaagaaaagcttcgcctttgacaacgtgggatacgagggaggactggatggactgggacctagctcccaggtggccacctctacagtccgaatcctgggcatgacttgccagagttgcgtgaaatcaattgaagaccggatcagtaatctgaagggaatcattagcatgaaagtgtccctggagcagggctcagccaccgtgaagtatgtccctagcgtggtctgcctgcagcaggtgtgccaccagatcggcgatatggggttcgaggcctccattgctgaagggaaagccgcttcttggcctagccggtccctgccagcacaggaagcagtggtcaagctgagagtggagggaatgacatgccagagctgcgtgagcagtatcgaaggaaaggtccgaaaactgcagggcgtggtccgggtgaaggtctctctgagtaaccaggaggccgtgattacctaccagccctatctgatccagcctgaagacctgagggatcacgtgaatgacatgggcttcgaggcagccatcaagtccaaagtggccccactgtctctggggcccattgacatcgaaagactgcagtccaccaacccaaagaggcccctgtcaagcgccaaccagaacttcaacaatagtgagaccctgggacaccagggctcacatgtggtcacactgcagctgaggattgacggcatgcactgcaagtcttgcgtgctgaacattgaggaaaatatcggccagctgctgggggtgcagtctatccaggtcagtctggagaacaagactgctcaggtgaaatacgatccttcatgcaccagcccagtggcactgcagcgcgctatcgaagcactgccccctggaaatttcaaggtgagcctgcctgacggagcagagggatccggaaccgatcacaggtcctctagttcacattccccaggatctccaccacgaaaccaggtgcagggaacatgttccaccacactgattgcaatcgccggcatgacttgcgcctcatgcgtgcacagcattgaagggatgatctctcagctggagggagtgcagcagatctcagtcagcctggccgagggcactgctaccgtgctgtacaatcccagtgtcatctcacctgaggaactgcgggctgcaattgaggacatggggttcgaagcttccgtggtctccgaatcttgcagtaccaaccccctggggaatcattccgccggaaactctatggtgcagactaccgacgggacacctacttctgtgcaggaggtcgcaccacacacaggacgcctgccagccaatcatgctcccgacatcctggccaaaagcccccagtccacccgggctgtggcacctcagaagtgttttctgcagatcaaaggcatgacctgcgcctcttgcgtgagcaacattgagcggaatctgcagaaggaagctggggtgctgagcgtgctggtcgcactgatggccggaaaggctgagatcaagtacgaccctgaagtgatccagccactggagattgcccagttcatccaggatctgggctttgaggccgctgtgatggaagactatgctgggagcgatggaaacattgaactgaccatcacaggcatgacttgtgcctcttgcgtgcacaacatcgagagtaaactgactagaaccaatgggattacctacgccagtgtggccctggctacatcaaaggctctggtgaaattcgaccccgagatcattggacctagggacatcatcaagatcattgaggaaatcggctttcacgcaagcctggcccagcgcaacccaaatgcccaccatctggaccataagatggagatcaagcagtggaagaaaagtttcctgtgctcactggtgtttggaatccccgtcatggccctgatgatctacatgctgatccctagcaacgagccacaccagtccatggtgctggatcataacatcattcctggcctgtccatcctgaatctgattttctttatcctgtgcacattcgtgcagctgctgggaggctggtacttttatgtgcaggcatataaatcactgcgacaccggagcgccaatatggacgtgctgattgtcctggcaacctctatcgcctacgtgtatagtctggtcatcctggtggtcgcagtggcagagaaggcagaacggagccccgtgactttctttgatacccctccaatgctgttcgtgtttatcgctctgggcagatggctggaacatctggcaaagtcaaaaaccagcgaggctctggcaaagctgatgagcctgcaggctaccgaagcaacagtggtcactctgggagaggacaacctgatcattcgcgaggaacaggtgcctatggaactggtccagcgaggcgacatcgtgaaggtggtcccagggggaaaattccccgtggacggcaaggtcctggaggggaatactatggccgatgaatccctgatcaccggcgaggctatgcctgtgacaaagaaaccaggatcaactgtcattgctggcagcatcaacgcacacgggtccgtgctgatcaaggccacacatgtcgggaatgacacaactctggctcagattgtgaaactggtcgaggaagcccagatgtccaaggctcctatccagcagctggccgatcggttctccggctacttcgtgcccttcatcattatcatgtctacactgactctggtggtctggattgtgatcggattcattgactttggcgtggtccagagatattttcccaaccctaataagcacatcagccagaccgaagtgatcatcaggttcgcatttcagaccagtattacagtgctgtgcatcgcctgcccatgttcactggggctggctacccccacagcagtgatggtcggaacaggggtggcagcacagaacggcatcctgatcaagggcgggaaacccctggagatggcccacaagatcaaaactgtgatgtttgacaaaactgggaccattacacatggagtgccacgcgtcatgcgagtgctgctgctgggcgatgtggcaaccctgcctctgagaaaggtcctggcagtggtcggaacagcagaggctagctccgaacacccactgggggtggccgtcacaaagtactgcaaagaggaactgggcactgagaccctggggtattgtactgacttccaggcagtgccaggatgcggaatcggatgtaaagtctctaacgtggaagggattctggctcacagtgagcggcccctgagcgcacctgcatcccatctgaatgaagcaggaagcctgccagcagagaaggacgctgtgcctcagaccttttccgtcctgatcggcaacagagaatggctgcggagaaatgggctgacaatttctagtgacgtgtccgatgccatgacagatcacgagatgaaaggccagactgcaattctggtggccatcgacggagtcctgtgcggcatgattgctatcgcagatgccgtgaagcaggaggctgcactggccgtccataccctgcagtctatgggcgtggacgtggtcctgatcaccggggataaccggaaaacagctagagcaattgccactcaagtgggcatcaataaggtgttcgctgaagtcctgcctagccacaaggtcgcaaaagtgcaggagctgcagaacaagggcaagaaagtcgccatggtgggagacggcgtgaatgatagcccagctctggcacaggcagacatgggagtcgctattgggacaggaactgacgtggcaatcgaggccgctgatgtggtcctgattaggaatgacctgctggatgtggtcgcttctattcatctgagtaagaggacagtgaggcgcattcgcatcaacctggtgctggccctgatctacaatctggtgggcatccccatcgcagcaggcgtgtttatgccaattgggatcgtcctgcagccctggatgggctcagctgcaatggccgcttcaagcgtgagcgtggtcctgtcctctctgcagctgaaatgctacaagaaaccagatctggagcggtacgaagctcaggcacacggacatatgaagcccctgaccgcttcccaggtgtctgtccacatcggcatggacgatagatggagggacagcccaagggcaactccatgggatcaggtcagttacgtgagccaggtcagcctgagttcactgaccagcgacaagccctcccgccattctgcagccgctgatgacgacggggacaagtggagcctgctgctgaacgggagggacgaagaacagtacatttga

SEQ ID NO: 31 - DNA, 4116 bp, Synthetic construct
  source 1..4116: mol_type = other DNA
taggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggagaagaggctgggagtcaagccaaatcctgcttcctggattttatcaggatattattggcagacatctgcgaagtggttgagaagcctgtacctgttttatacttgcttttgcttcagcgttctgtggttgtcaacagatgccagtgagagcaggtgccagcaggggaagacacaatttggagttggcctgagatctgggggagaaaatcacctctggcttcttgaaggaaccccctctctccagtcatgttgggctgcctgctgccaggactctgcctgccatgtcttttggtggctagaagggatgtgcattcaggcagactgcagcaggccccagagctgccgggcttttaggacacactcctccaattccatgctggtgtttttaaaaaaattccaaactgcagatgatttgggctttctacctgaagatgatgtaccacatcttctggggctaggttggaactgggcatcttggaggcagagcccacccagagctgcactcagacctgctgtatcttccagtgaccagcagagcttaatcaggaagcttcagaagagaggtagtcccagtgacgtagttacacctatagtgacacagcattctaaagtgaatgactccaacgaattaggtggtctgactaccagtggctctgcagaggtccacaaggcgattacaatttccagtcccctaaccacagacctgactgcagagctgtctggtgggccaaagaatgtatcagtgcaacctgaaatatcagagggtcttgctactacgcccagcactcaacaagtaaaaagttctgagaaaacccagattgctgtcccccagccagtggctccctcctacagttatgctacccctaccccccaggcctctttccagagcacctcagcaccatacccagttataaaggaactggtggtatctgctggagagagtgtccagataaccctgcctaagaatgaagttcaattaaatgcatatgttctccaagaaccacctaaaggagaaacctacacctacgactggcagctgattactcatcctagagactacagtggagaaatggaagggaaacattcccagatcctcaaactatcgaagctcactccaggcctgtatgaattcaaagtgattgtagagggtcaaaatgcccatggggaaggctatgtgaacgtgacagtcaagccagagccccgtaagaatcggccccccattgctattgtgtcacctcagttccaggagatctctttgccaaccacttctacagtcattgatggcagtcaaagcactgatgatgataaaatcgttcagtaccattgggaagaacttaaggggcctctaagagaagagaagatttctgaagatacagccatattaaaactaagtaaactcgtccctgggaactacactttcagcttgactgtagtagactctgatggagctaccaactctactactgcaaacctgacagtgaacaaagctgtggattacccccctgtggccaacgcaggccccaaccaagtgatcaccctgccccaaaactccatcaccctctttgggaaccagagcactgatgatcatggcatcaccagctatgagtggtcactcagcccaagcagcaaagggaaagtggtggagatgcagggtgttagaacaccaaccttacagctctctgcgatgcaagaaggagactacacttaccagctcacagtgactgacacaataggacagcaggccactgctcaagtgactgttattgtgcaacctgaaaacaataagcctcctcaggcagatgcaggcccagataaagagctgacccttcctgtggatagcacaaccctggatggcagcaagagctcagatgatcagaaaattatctcatatctctgggaaaaaacacagggacctgatggggtgcagctcgagaatgctaacagcagtgttgctactgtgactgggctgcaagtggggacctatgtgttcaccttgactgtcaaagatgagaggaacctgcaaagccagagctctgtgaatgtcattgtcaaagaagaaataaacaaaccacctatagccaagataactgggaatgtggtgattaccctacccacgagcacagcagagctggatggctctaagtcctcagatgacaagggaatagtcagctacctctggactcgagatgaggggagcccagcagcaggggaggtgttaaatcactctgaccatcaccctatcctttttctttcaaacctggttgagggaacctacacttttcacctgaaagtgaccgatgcaaagggtgagagtgacacagaccggaccactgtggaggtgaaacctgatcccaggaaaaacaacctggtggagatcatcttggatatcaacgtcagtcagctaactgagaggctgaaggggatgttcatccgccagattggggtcctcctgggggtgctggattccgacatcattgtgcaaaagattcagccgtacacggagcagagcaccaaaatggtattttttgttcaaaacgagcctccccaccagatcttcaaaggccatgaggtggcagcgatgctcaagagtgagctgcggaagcaaaaggcagactttttgatattcagagccttggaagtcaacactgtcacatgtcagctgaactgttccgaccatggccactgtgactcgttcaccaaacgctgtatctgtgaccctttttggatggagaatttcatcaaggtgcagctgagggatggagacagcaactgtgagtggagcgtgttatatgttatcattgctacctttgtcattgttgttgccttgggaatcctgtcttggactgtgatctgttgttgtaagaggcaaaaaggaaaacccaagaggaaaagcaagtacaagatcctggatgccacggatcaggaaagcctggagctgaagccaacctcccgagcaggcatcaaacagaaaggccttttgctaagtagcagcctgatgcactccgagtcagagctggacagcgatgatgccatctttacatggccagaccgagagaagggcaaactcctgcatggtcagaatggctctgtacccaacgggcagacccctctgaaggccaggagcccgcgggaggagatcctgtaggattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 32 - DNA, 32 bp, Synthetic construct
  source 1..32: mol_type = other DNA
tagagggtatataatggaagctcgacttccag

SEQ ID NO: 33 - skipped

SEQ ID NO: 34 - protein, 272 aa, Synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 35 - protein, 753 aa, Synthetic construct
MAPKKKRKVGIHGVPAALEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTSKRPAATKKAGQAKKKKGSYPYDVPDYALEEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 36 - protein, 753 aa, Synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSYPYDVPDYALEEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 37 - protein, 272 aa, Synthetic construct
MAPKKKRKVGIHGVPAALEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 38 - protein, 272 aa, Synthetic construct; identical to SEQ ID NO: 34

SEQ ID NO: 39 - protein, 580 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 40 - protein, 394 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDK

SEQ ID NO: 41 - protein, 580 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 42 - protein, 394 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 43 - protein, 388 aa, Synthetic construct
MQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 44 - protein, 479 aa, Synthetic construct
MQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMSGLEMADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 45 - protein, 476 aa, Synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 46 - protein, 554 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 47 - protein, 362 aa, Synthetic construct
MRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 48 - protein, 479 aa, Synthetic construct
MRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCALEMSGLEMADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 49 - protein, 476 aa, Synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCA

SEQ ID NO: 50 - protein, 559 aa, Synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRRDELNVHLRIHTGHKPFQCRICMRSFSRSDHLTNHIRTHTGEKPFACEFCGRKFARSDDLVRHAKIHLKQKEHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEKKAEKGGAPSASSAPPVSLAPVVTTCA

SEQ ID NO: 51 - protein, 631 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 52 - protein, 719 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRRDELNVHIRIHTGQKPFQCRICMRNFSRSDHLTNHIRTHTGEKPFACDICGRKFARSDDLVRHTKIHLRQKDRPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 53 - protein, 627 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHIRTHTGEKPFACDICGRKFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 54 - protein, 631 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 55 - protein, 473 aa, Synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEKKAEKGGAPSASSAPPVSLAPVVTTCA

SEQ ID NO: 56 - protein, 719 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSDPGALVRHIRIHTGQKPFQCRICMRNFSRSDNLVRHIRTHTGEKPFACDICGRKFAQSGDLRRHTKIHLRQKDRPYACPVESCDRRFSTHLDLIRHIRIHTGQKPFQCRICMRNFSTSGNLVRHIRTHTGEKPFACDICGRKFARSDNLVRHTKIHLRQKDRPYACPVESCDRRFSQSGHLTEHIRIHTGQKPFQCRICMRNFSERSHLREHIRTHTGEKPFACDICGRKFAQAGHLASHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 57 - protein, 627 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLTRHIRIHTGQKPFQCRICMRNFSHSTTLTNHIRTHTGEKPFACDICGRKFARSDNRKTHIRTHTGEKPFACDICGRKFSTSHSLTEHIRIHTGQKPFQCRICMRNFSQSSSLTRHIRTHTGEKPFACDICGRKFARSDNRKTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 58 - protein, 473 aa, Synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCA

SEQ ID NO: 59 - protein, 627 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLTRHIRIHTGQKPFQCRICMRNFSRSDNLTTHIRTHTGEKPFACDICGRKFARSDERKRHIRTHTGEKPFACDICGRKFSQSGNLTEHIRIHTGQKPFQCRICMRNFSTSGHLTRHIRTHTGEKPFACDICGRKFAQSSTRKEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 60 - protein, 627 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHIRTHTGEKPFACDICGRKFSQSGNLTEHIRIHTGQKPFQCRICMRNFSTSGHLVRHIRTHTGEKPFACDICGRKFAQNSTLTEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 61 - protein, 272 aa, Synthetic construct
MAPKKKRKVGIHGVPAALEPGEKPYKCPECGKSFSSPADLTRHQRTHTGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 62 - protein, 272 aa, Synthetic construct
MAPKKKRKVGIHGVPAALEPGEKPYKCPECGKSFSDPGALVRHQRTHTGEKPYKCPECGKSFSRSDNLVRHQRTHTGEKPYKCPECGKSFSQSGDLRRHQRTHTGEKPYKCPECGKSFSTHLDLIRHQRTHTGEKPYKCPECGKSFSTSGNLVRHQRTHTGEKPYKCPECGKSFSRSDNLVRHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 63 - protein, 261 aa, Synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSDALDDFDLDMLGSDALDDFDLDMLGSDALDDFDLDMLGSDALDDFDLDML

SEQ ID NO: 64 - protein, 386 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 65 - protein, 402 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 66 - protein, 569 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 67 - protein, 585 aa, Synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 68 - protein, 402 aa, Synthetic construct; identical to SEQ ID NO: 65

SEQ ID NO: 69 - protein, 569 aa, Synthetic construct; identical to SEQ ID NO: 66

SEQ ID NO: 70 - protein, 585 aa, Synthetic construct; identical to SEQ ID NO: 67

SEQ ID NO: 71 - protein, 16 aa, Synthetic construct
MELELDAGDQDLLAFL

SEQ ID NO: 72 - protein, 530 aa, Synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDVYVGGLESRVLKYTAQNMELQNKVQLLEEQNLSLLDQLRKLQAMVIEISNKTSSSSTCILVLLVSFCLLLVPAMYSSDTRGSLPAEHGVLSRQLRALPSEDPYQLELPALQSEVPKDSTHQWLDGSDCVLQAPGNTSCLLHYMPQAPSAEPPLEWPFPDLFSEPLCRGPILPLQANLTRKGGWLPTGSPSVILQDRYSG

SEQ ID NO: 73 - protein, 530 aa, Synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDVYVGGLESRVLKYTAQNMELQNKVQLLEEQNLSLLDQLRKLQAMVIEISNKTSSSSTCILVLLVSFCLLLVPAMYSSDTRGSLPAEHGVLSRQLRALPSEDPYQLELPALQSEVPKDSTHQWLDGSDCVLQAPGNTSCLLHYMPQAPSAEPPLEWPFPDLFSEPLCRGPILPLQANLTRKGGWLPTGSPSVILQDRYSG

SEQ ID NO: 74 - protein, 380 aa, Synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRLEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSVYVGGLESRVLKYTAQNMELQNKVQLLEEQNLSLLDQLRKLQAMVIEIS

SEQ ID NO: 75 - protein, 380 aa, Synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDVYVGGLESRVLKYTAQNMELQNKVQLLEEQNLSLLDQLRKLQAMVIEIS

SEQ ID NO: 76 - protein, 50 aa, Synthetic construct
DALDDFDLDMLGSDALDDFDLDMLGSDALDDFDLDMLGSDALDDFDLDML

SEQ ID NO: 77 - protein, 523 aa, Synthetic construct
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 78 - protein, 275 aa, Synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 79 - protein, 274 aa, Synthetic construct
SGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 80 - protein, 184 aa, Synthetic construct
MADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 81 - protein, 184 aa, Synthetic construct
AADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 82 - protein, 543 aa, Synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 83 - protein, 387 aa, Synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKAEKGGAPSASSAPPVSLAPVVTTCA

SEQ ID NO: 84 - protein, 261 aa, Synthetic construct; identical to SEQ ID NO: 63

SEQ ID NO: 85 - protein, 7 aa, Synthetic construct
RSDNLVR

SEQ ID NO: 86 - protein, 7 aa, Synthetic construct
REDNLHT

SEQ ID NO: 87 - protein, 7 aa, Synthetic construct
RSDELVR

SEQ ID NO: 88 - protein, 7 aa, Synthetic construct
QSGNLTE

SEQ ID NO: 89 - protein, 7 aa, Synthetic construct
TSGHLVR

SEQ ID NO: 90 - protein, 7 aa, Synthetic construct
QNSTLTE

SEQ ID NO: 91 - DNA, 69 bp, Synthetic construct
  source 1..69: mol_type = other DNA
accgaaccccgcgtttatgaacaaacgacccaacaccgtgcgttttattctgtctttttattgccgtca

SEQ ID NO: 92 - protein, 1049 aa, Synthetic construct
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 93 - protein, 176 aa, Synthetic construct
LEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTS

SEQ ID NO: 94 - DNA, 3872 bp, Synthetic construct
  source 1..3872: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggcgcgccggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctggaattcgccaccatggccccaaagaagaagcggaaggtcggtatccacggagtcccagcagccctcgaaccaggtgaaaaaccttacaaatgtcctgaatgtgggaaatcattcagtcgcagcgacaacctggtgagacatcaacgcacccatacaggagaaaaaccttataaatgtccagaatgtggaaagtccttctcacgagaggataacttgcacactcatcaacgaacacatactggtgaaaaaccatacaagtgtcccgaatgtggtaaaagttttagccggagcgatgaacttgtccgacaccaacgaacccatacaggcgagaagccttacaaatgtcccgagtgtggcaagagcttctcacaatcagggaatctgactgagcatcaacgaactcataccggggaaaaaccttacaagtgtccagagtgtgggaagagcttttccacaagtggacatctggtacgccaccagaggacacatacaggggagaagccctacaaatgccccgaatgcggtaaaagtttctctcagaatagtaccctgaccgaacaccagcgaacacacactgggaaaaaaacgagtaaaaggccggcggccacgaaaaaggccggccaggcaaaaaagaaaaagggatccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataaaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctaagcttgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 95 - protein, 176 aa, Synthetic construct
LEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTS

SEQ ID NO: 96 - DNA, 2867 bp, Synthetic construct
  source 1..2867: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtggcgcgccgtttaaacttaattaagctagccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgaattcgccaccatggccccaaagaagaagcggaaggtcggtatccacggagtcccagcagcccttgagcccggtgagaagccttataaatgccctgaatgcgggaaaagcttcagccgctcagacaacctcgttcgacaccagcggacacataccggtgaaaagccatacaaatgcccagaatgtgggaagtcattctcccaccggactacactcacgaaccatcagcgcactcatacgggagagaaaccctataagtgcccggaatgcgggaaatcattcagtagagaagacaatctccatactcatcaacgaactcacacaggcgagaagccatacaagtgcccagaatgcgggaaatcattttctaccagccattctctcactgaacaccagagaacccacacgggggaaaagccgtacaagtgtccagagtgtggaaagtccttcagccagtctagctcactggtgaggcatcagaggactcataccggcgaaaaaccctataagtgccccgagtgtgggaaaagtttttccagggaggataacctgcatacgcatcaaaggacccataccggtaaaaaaacatccaaaaggccggcggccacgaaaaaggccggccaggcaaaaaagaaaaagggatcctacccatacgacgtaccagattacgctctcgaggacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataaactagtaataaaagatctttattttcattagatctgtgtgttggttttttgtgtgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg